(19) United States
(12) Patent Application Publication
(10) Pub. No.: US 2026/0000697 A1
(43) Pub. Date: Jan. 1, 2026

(54) COMBINATION FORMULATION OF CEDAZURIDINE

(71) Applicant: Otsuka Pharmaceutical Co., Ltd., Tokyo (JP)

(72) Inventors: Nipun Davar, Pleasanton, CA (US); Jim Kou, Pleasanton, CA (US); Rachna Jain, Pleasanton, CA (US)

(21) Appl. No.: 18/840,444

(22) PCT Filed: Feb. 22, 2023
(86) PCT No.: PCT/US2023/013614

§ 371 Date: Aug. 21, 2024

Related U.S. Application Data
(60) Provisional application No. 63/312,714, filed on Feb. 22, 2022.
(60) Provisional application No. 63/312,712, filed on Feb. 22, 2022.
(60) Provisional application No. 63/377,312, filed on Sep. 27, 2022.

(51) Int. Cl. A61K 31/7068; A61K 9/20; A61K 9/28; A61K 9/48; A61K 31/407; A61K 31/4178; A61K 31/4184; A61K 31/439; A61K 31/501; A61K 31/506; A61K 31/553; A61K 31/675; A61K 31/706; A61K 33/243

(52) U.S. Cl. CPC A61K 31/7068; A61K 9/2009; A61K 9/2013; A61K 9/2018; A61K 9/2054; A61K 9/2846; A61K 9/2866; A61K 9/2886; A61K 9/4808; A61K 9/485; A61K 9/4858; A61K 9/4866; A61K 31/407; A61K 31/4178; A61K 31/4184; A61K 31/439; A61K 31/501; A61K 31/506; A61K 31/553; A61K 31/675; A61K 31/706; A61K 33/243

(57) ABSTRACT

The present disclosure relates generally to pharmaceutical dosage forms comprising azacitidine and cedazuridine.

### CROSS REFERENCE TO RELATED APPLICATIONS

This application is a national stage of International Application No. PCT/US2023/013614 filed Feb. 22, 2023, which claims the benefit under 35 U.S.C. § 119(e) of U.S. Provisional Application No. 63/312,712 filed Feb. 22, 2022, U.S. Provisional Application No. 63/312,714 filed Feb. 22, 2022, and U.S. Provisional Application No. 63/377,312 filed Sep. 27, 2022, all of which are hereby incorporated by reference herein in their entireties.

### FIELD

This disclosure relates to combination formulations comprising azacitidine, or a pharmaceutically acceptable salt thereof, and cedazuridine, or a pharmaceutically acceptable salt thereof.

### BACKGROUND

Cancer is a worldwide health problem; the World Health Organization estimates that cancer accounted for nearly 10 million deaths worldwide in 2020. Every cancer type requires a specific treatment regimen and sometimes cancers acquire resistance to certain treatments. Combination therapies can reduce development of resistance and improve overall survival (OS) and/or delay disease progression (progression free survival (PFS)).

It has been shown that cancers that acquire resistance to cytidine analog drugs often overexpress cytidine deaminase (CDA) (Leuk. Res. 1990, 14, 751-754). Leukemic cells expressing a high level of CDA can become resistant to cytosine antimetabolites and thereby limit the antineoplastic activity of such therapeutics (Biochem. Pharmacol. 1993, 45, 1857-1861). Inhibitors of CDA are useful in combination chemotherapy regimens involving cytidine analog drugs.

It has been found that when azacitidine, a cytidine analog drug, is orally administered (8 mg/kg) to repeatedly phlebotomized baboon (PCV less than 20%) there is no elevation in the fetal hemoglobin levels (Hb F), indicating very minimal oral bioavailability (DeSimone et al (1985) Amer. J. of Hem. 18:283-288). The poor bioavailability of cytidine analogs is presumably due to the degradation of the cytidine analog by cytidine deaminases as well as their inherent chemical instability in the acidic gastric environment. To date, only one orally administered azacitidine product has been approved, and provides a mean oral bioavailability of approximately 11% relative to subcutaneous administration. There is a need for formulations of cytidine analogs such as azacitidine which can increase bioavailability of azacitidine.

Fixed dose combination (FDC) formulations are also desirable because they can reduce the pill burden on a patient undergoing combination chemotherapy and improve patient compliance with a treatment regimen. There is need for FDC formulations comprising combinations of cytidine analogs and CDA inhibitors.

### SUMMARY

It had been reported that certain cytidine analogs (e.g. 5-azacytidine) is acid-labile and enteric coating of such drugs has been introduced. See, e.g., US2004/0162263. It has now been discovered that enteric release of azacitidine increases bioavailability of azacitidine and co-administration with cedazuridine, a CDA inhibitor, further increases bioavailability of azacitidine. Gastric release of cedazuridine further enhances the effect of increasing the absorption of azacitidine and the amount of oral azacitidine can be reduced. The present disclosure provides a fixed dose combination (FDC) formulation that enables immediate release of cedazuridine and delayed, enteric release of azacitidine, thereby achieving a desired level of azacitidine bioavailability with a reduced amount of azacitidine.

The present disclosure, in one embodiment, provides a pharmaceutical dosage form comprising cedazuridine, or a pharmaceutically acceptable salt thereof, and azacitidine, or a pharmaceutically acceptable salt thereof, wherein the cedazuridine is formulated for immediate release; at least a portion of the azacitidine is formulated for modified release and provided as enteric-coated minitablets or pellets. In some embodiments, the cedazuridine is formulated for immediate release. In some embodiments, substantially all of the azacitidine is configured to be released outside the stomach.

Provided herein is a pharmaceutical dosage form comprising cedazuridine, or a pharmaceutically acceptable salt thereof, and azacitidine, or a pharmaceutically acceptable salt thereof, wherein the cedazuridine is formulated for immediate release; and the azacitidine is formulated as modified release minitablets with an enteric coat. In some embodiments, the cedazuridine is provided as uncoated minitablets, pellets, or powder.

Provided herein is a capsule comprising one or more azacitidine minitablets formulated for modified release and immediate release cedazuridine in a form of uncoated powder or minitablet. The azacitidine minitablets comprise azacitidine or a pharmaceutically acceptable salt thereof, pharmaceutically acceptable excipients, and an enteric coat. The immediate release cedazuridine comprises cedazuridine or a pharmaceutically acceptable salt thereof and pharmaceutically acceptable excipients.

Provided herein is a method of treating cancer in a patient comprising administering the pharmaceutical dosage form. Also provided herein is the pharmaceutical dosage form for use in the treatment of cancer, or for use in manufacturing a medicament for the treatment of cancer. Also provided herein is use of the pharmaceutical dosage form for the treatment of cancer, or for the manufacture of medicament for the treatment of cancer.

### BRIEF DESCRIPTION OF THE DRAWINGS

FIG. 1 shows the dissolution release profile of azacitidine from a coated FDC tablet comprising cedazuridine and azacitidine with 12% or 15% (coating) weight gain according to one embodiment, at pH 6.8.

FIG. 2 shows the dissolution release profile for azacitidine from a capsule comprising uncoated or coated azacitidine minitablets, according to one embodiment, at pH 6.8.

FIG. 3 shows the dissolution profile for azacitidine from a capsule comprising azacitidine minitablets, where 50% of azacitidine (10 mg) is uncoated (immediate release) and 50% azacitidine (10 mg) is coated (delayed release), according to one embodiment, at pH 6.8.

FIG. 4 shows the dissolution profile for azacitidine from a capsule comprising azacitidine minitablets, where uncoated and coated azacitidine minitablets are in ratios of 1:1 or 1:2, according to one embodiment, at pH 6.8.

FIG. 5 shows the dissolution profile for azacitidine from a capsule comprising azacitidine minitablets with 15% or 20% (coating) weight gain, according to one embodiment, at pH 6.8.

FIG. 6 shows the dissolution release profile for azacitidine from a capsule comprising uncoated cedazuridine minitablets and 75:25 and 80:20 ethyl cellulose-coated azacitidine minitablets comprising intragranular and extragranular layers, according to one embodiment, at pH 6.8. (e.g. refers to weight gain from the coating).

FIG. 7 shows dissolution data for coated azacitidine minitablets according to one embodiment, in going from pH 1 to pH 6.8.

FIG. 8 shows dissolution data for coated azacitidine minitablets according to one embodiment, in going from pH 1 to pH 6.8.

FIG. 9 shows dissolution profile for immediate release cedazuridine minitablets according to one embodiment, in going from pH 1 to pH 6.8.

FIG. 10 shows dissolution profile for coated azacitidine minitablets according to one embodiment, in going from pH 1 to pH 6.8.

FIG. 11 shows dissolution profile for coated azacitidine minitablets according to one embodiment, at pH 2.3, 3.0, 4.5, 5.2, 5.5, and 6.0.

FIG. 12 shows azacitidine mean concentration time profile for three groups on Day 1 and Day 2 in a monkey pharmacokinetics (PK) study.

FIG. 13 shows cedazuridine mean concentration time profile for three groups on Day 1 and Day 2 in a monkey pharmacokinetics (PK) study.

FIG. 14 shows azacitidine mean concentration time profile for three groups on Day 1 and Day 2 in a monkey pharmacokinetics (PK) study.

FIG. 15 shows azacitidine mean concentration profile for 15% wt. gain coated minitablets at 80/20 ratio from the Part 1 PK study.

FIG. 16 shows azacitidine mean concentration profile for 20% wt. gain coated minitablets at 75/25 ratio from the Part 2 PK study.

FIG. 17 shows azacitidine mean concentration profile for 5.3% Eudragit® coated azacitidine minitablets from a monkey PK study.

FIG. 18A shows a dissolution profile for cedazuridine from a capsule according to one embodiment.

FIG. 18B shows a dissolution profile for azacitidine from a capsule according to one embodiment.

FIG. 19 shows plasma exposure for cedazuridine from a pharmacokinetics (PK) study in monkeys.

FIG. 20 shows plasma exposure for azacitidine from a PK study in monkeys.

It will be recognized that some or all of the figures are schematic representations for purpose of illustration.

### DETAILED DESCRIPTION

Efforts to increase bioavailability of cytosine analogs have been described in, for example, U.S. Patent Application Publication No. 2004/0162263 (Sands, et al.). In this publication, delivery of decitabine in an enteric-coated formulation is disclosed such that the drug is preferably absorbed in the upper regions of the small intestine, such as the jejunum. U.S. Patent Application Publication No. 2008/0057086 (Pharmion) describes delivery of azacitidine to the upper regions of the large intestine. Bioavailability for an 80 mg oral dose in 3 patients relative to SC dosing was improved by 6.3%, 24%, and 22%. Tₘₐₓ for the 3 subjects who received an 80 mg dose occurred at 1.5, 2.0, and 1.0 h post-dose.

The stomach is a region of high acidity (about pH 1 to 3). Specific glands and organs emptying into the small intestine raise the pH of the material leaving the stomach to approximately pH 6.0 to 6.5. The large intestine and the colon are typically at about pH 6.4 to 7.0. The transit time through the small intestine is approximately three hours. In contrast, the transit time through the large intestine is approximately 35 hours. Stomach acid is believed to degrade azacitidine. Further, a longer transit time in the large intestine may lead to higher enzymatic degradation of azacitidine and consequent poor bioavailability. Accordingly, release and absorption of azacitidine outside the stomach is desirable to achieve higher bioavailability. In some embodiments, co-administration of cedazuridine, a cytidine deaminase inhibitor, may reduce enzymatic degradation of azacitidine. In some embodiments, release of azacitidine at a pH of about 6.8 in the proximal regions of the small intestine may increase absorption of azacitidine.

In some embodiments, the compositions described herein enhance bioavailability of azacitidine irrespective of the location of the release of azacitidine in the intestine. In some of such embodiments, cedazuridine is co-administered and/or co-formulated with azacitidine. Further, the location of release of cedazuridine affects the enhancement of bioavailability of azacitidine. In some embodiments, immediate release of cedazuridine in the stomach enhances bioavailability of azacitidine to a higher extent compared to delayed release of cedazuridine when cedazuridine and azacitidine are administered in a fixed dose composition described herein.

Azacitidine bioavailability is further enhanced by the modified-release dosage forms of azacitidine described herein. Further, variability in drug exposure is reduced. In some embodiments, the azacitidine is released at pH>3. In some embodiments, the azacitidine is released outside the stomach. In some embodiments, enhanced absorption and/or bioavailability of azacitidine may occur when cedazuridine is co-administered and released prior to release of azacitidine. For example, cedazuridine may be released in the stomach and azacitidine may be released outside the stomach, thereby improving absorption and/or bioavailability of azacitidine.

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. The term “about X” includes description of “X”.

The singular forms “a” and “the” include plural references unless the context clearly dictates otherwise. Thus, e.g., reference to “the dosage form” includes a plurality of such dosage forms.

The terms “optional” or “optionally” means that the subsequently described event or circumstance may or may not occur, and that the description includes instances where said event or circumstance occurs and instances in which it does not.

The term “polymer” refers to a chemical compound or mixture of compounds consisting of repeating structural units created through a process of polymerization. Suitable polymers useful in the present dosage forms are described throughout.

A “blend” refers to a solid form wherein an active agent is mixed with additional excipients including and not limited to a bulk filler, a glidant, and/or a lubricant. The ratio of the active agent to the excipients may vary and depends on the properties of the active agent. Typically a blend is in the form of a powder.

In some embodiments, a “granule” is a particle which has an irregular shape. In some embodiments, “granules” are solid agglomerates of powder particles.

A “pellet” is a solid mass comprising an active agent and/or additional fillers. Pellets include and are not limited to discs, beads, prolate spheroids, oblate spheroids, spheroids, cylinders, and the like. A “minitablet” is a rounded compressed solid dosage forms with a diameter smaller than tablets (i.e. equals to or less than 6 mm diameter), compressed solid mass comprising an active agent and/or additional fillers, In some embodiments, “pellet” includes “minitablet,” or vice versa, In some embodiments, “pellet” and “minitablet” may be used interchangeably. A “pellet” and/or a “minitablet” may refer to a subunit of a unit pharmaceutical dosage form, such as a capsule or a tablet.

In some embodiments, a “tablet” is a cylinder of a compressed solid mass, and typically the height of the cylinder is less than or equal to the diameter of the cylinder. In some embodiments, a “tablet” may refer to a pharmaceutical dosage form, which may include one or more subunits such as pellets, minitablets, granules, or powders.

A “FDC” or “fixed dose combination” refers to a pharmaceutical dosage form containing two or more drugs contained in a single dosage form, such as a capsule or tablet.

“Modified release” refers to release of a drug that occurs substantially outside of the stomach. In some embodiments, modified release is not sensitive to pH variations that occur outside of the stomach. By way of example only, hydroxypropyl methylcellulose coatings are substantially insensitive to pH variations outside of the stomach and modified release may occur at any pH higher than the pH in the stomach. In some embodiments, modified release is pH-sensitive and release at a desired pH is achieved by use of suitable coatings. By way of example only, polymethacrylate polymer coatings such as Eudragit® are tunable and release can be modified to occur at pH>5.5 (duodenum targeting); at pH 6-7 (jeunum); or at a pH>7 (ileum and colon).

“Enteric release” refers to release of a drug substantially in the intestine to prevent the degradation of the drug from acid-catalyzed hydrolysis in the stomach. “Enteric coat” or “enteric coating” refers to a coating that allows for release of a drug substantially in the intestine. In some embodiments, enteric release is “delayed release” or “timed release” which is pH-sensitive and occurs at pH>5.5 (duodenum targeting); at pH 6-7 (jejunum); or at a pH>7 (ileum and colon). “Delayed release coating” or “delayed release coat” refers to a coating that is sensitive to pH variations in the intestine and release of a drug is delayed till the target pH-I environment is available. In some embodiments, an enteric coating is a delayed release coating.

“Seal coat” refers to a coating which is layered onto an uncoated pellet prior to coating it with an enteric coating. The seal coat forms an intermediate layer and prevents interaction between the core comprising the active agent and the enteric coat. In some embodiments, the seal coat comprises a polymer that allows for intermediate release.

The term “% w/w” of a component as used herein refers to the weight of the component based on the total weight of a dosage form comprising the component. For example, if component A is present in an amount of 50% w/w in a 100 mg dosage form, component A is present in an amount of 50 mg.

The term “bulk fillers” refers to chemical compounds that are used to dilute the compound of interest. Bulk fillers can also serve to stabilize compounds. Non-limiting examples of bulk fillers include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “glidant” refers to an excipient used to promote powder flow by reducing interparticle friction and cohesion. Glidants may improve flow-properties during tablet compression and produce an anti-caking effect. Non-limiting examples of glidants include colloidal silicon dioxide, talc, fumed silica, starch, starch derivatives, magnesium carbonate, and bentonite. Glidants are typically used in conjunction with lubricants.

The term “lubricant” refers to an excipient which is added to a powder blend to prevent the compacted powder mass from sticking to the equipment during processing. It aids the ejection of a compacted solid mass from dies, and can improve powder flow. Non-limiting examples of lubricants include magnesium stearate, stearic acid, silica, fats, calcium stearate, polyethylene glycol, sodium stearyl fumarate, talc, and solubilizers such as fatty acids including lauric acid, oleic acid, and C8/C10 fatty acids.

“Substantially” all of the azacitidine means greater than 70%; or greater than 75%; or greater than 80%; or greater than 85%, or greater than 90%; or greater than 95%; or greater than 99% of the azacitidine. Release of a drug “substantially” outside the stomach means greater than 70%; or greater than 75%; or greater than 80%; or greater than 85%, or greater than 90%; or greater than 95%; or greater than 99% of the drug is released outside of the stomach.

“pH variations in the intestine” refers to the changes in pH within the intestinal lumen in going from the stomach to the rectum. For instance, pH in the duodenum is about 5.5 or higher; pH in the jejunum is about 6-7; pH in the ileum and colon is about 7 or higher; pH in the cecum is about 5.7; pH in the rectum is about 6.7.

An enteric coating which is “not sensitive to pH” refers to a coating that may allow for release of a drug at any pH that occurs outside the stomach. An enteric coating which is “sensitive to pH” may allow for release of a drug at a targeted pH, for example a targeted area within the intestinal lumen.

As used herein, “hydroxypropyl methylcellulose” is used interchangeably with “HPMC” and/or “Hypromellose”. “HPMC” includes Hypromellose acetate succinate (HPMCAS) and/or grades of HPMCAS which are commercially available.

As used herein Eudragit® refers to a class of polymethacrylate-based copolymers. It includes anionic, cationic, and/or neutral copolymers based on methacrylic acid and methacrylic/acrylic esters or their derivatives. Various grades of the polymers are commercially available including and not limited to L 30 D-55, FS 30 D, and FL 30 D-55. Anionic Eudragit® L dissolves at pH>6 and is used for enteric coating, while Eudragit® ?) S, soluble at pH>7 is used for colon targeting. Combinations of Eudragit® S and Eudragit® L can provide drug release at pH<7, Eudragit® RL and RS, having quaternary ammonium groups, are water insoluble, but swellable/permeable polymers which are suitable for the sustained release film coating applications.

“Cedazuridine” includes epimers of cedazuridine and is not limited to the isomer drawn herein.

“Amino acid salts” in the context of buffers refers to buffer salts comprising one or more amino acids, e.g., histidine, glycine, or any other amino acid known to one of skill in the art. Amino acids contain positively charged amino groups and negatively charged carboxyl groups. The charged regions of these molecules can bind hydrogen and hydroxyl ions, and thus function as buffers.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkyl” refers to an alkyl group as defined above and “haloalkoxy” refers to an alkoxy group as defined above, wherein one or more hydrogen atoms of the alkyl or alkoxy group are replaced by a halogen.

As used herein, the term “amino” refers to amine of formula —N(R^{N})₂, where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, —NH₂, —NH(alkyl), —N(alkyl)₂, alkyl, alkenyl, alkynyl, haloalkyl, alkoxy, or haloalkoxy).

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Halogen” or “halo” includes fluoro, chloro, bromo, and iodo.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e. the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g. 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Hydroxy” or “hydroxyl” refers to the group —OH.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

“Medicament” or “medicaments” as referred to herein may be prepared by conventional processes, including the combination of one or more compounds according to the present disclosure and a pharmaceutically acceptable carrier. For example, in the context of this disclosure, medicament may include both azacitidine and cedazuridine.

### Combinations of Azacitidine and Cedazuridine

In one embodiment, provided herein is a pharmaceutical dosage form or composition comprising azacitidine or a pharmaceutically acceptable salt thereof. The pharmaceutical dosage form or composition may further include a CDA inhibitor, such as cedazuridine, or a pharmaceutically acceptable salt thereof, such that the bioavailability of azacitidine is enhanced.

In one embodiment, provided herein is a fixed dose combination, a pharmaceutical dosage form or a composition comprising cedazuridine, or a pharmaceutically acceptable salt thereof, and azacitidine, or a pharmaceutically acceptable salt thereof. The chemical structures of both cedazuridine and azacitidine are shown below.

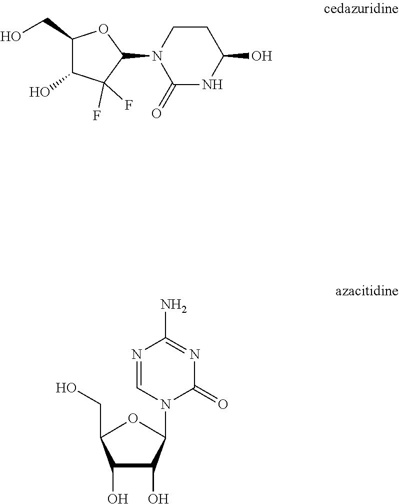

### Release Profile of Azacitidine

It has now been discovered that enteric release of azacitidine in the intestine is more advantageous for the bioavailability of azacitidine, as compared to gastric release, and at least a portion of azacitidine in the pharmaceutical dosage form or composition may be formulated such that it is released in intestine, not in the stomach. In one embodiment, a fixed dose combination, a pharmaceutical dosage form or a composition comprises azacitidine, or a pharmaceutically acceptable salt thereof, wherein at least a portion of the azacitidine is formulated for modified release. For example, about 5%, 10%, 15%, 20%, 25%, 30%, 33.3%, 35%, 40%, 45%, 50%, 55%, 60%, 65%, 70%, 75%, 80%, 85%, 90%, 95% or 100% of the azacitidine, by weight, may be formulated for modified release. In some embodiments, about ⅓, or ⅔ of the azacitidine, by weight based on the total weight of the azacitidine in the tablet or capsule, may be formulated for modified release. In some embodiments, none of the azacitidine is formulated for immediate release (e.g., release in the stomach) and the azacitidine is formulated for modified release. In some of such embodiments, the azacitidine formulated for modified release is coated. In some embodiments, the coated azacitidine is in the form of pellets. In some embodiments, the coated azacitidine is in the form of minitablet.

In some embodiments of the pharmaceutical dosage form, the portion of azacitidine that is formulated for modified release is formulated for enteric release. In some of such embodiments, the portion of the azacitidine that is formulated for modified release is formulated for enteric release that is delayed release at a targeted pH (e.g. pH of duodenum, the jejunum, the ileum, or the colon). In some embodiments of the pharmaceutical dosage form, the portion of azacitidine that is formulated for immediate release is provided as uncoated minitablets or pellets. Such uncoated minitablets or pellets form cores which are optionally coated with release modifying coatings. In some embodiments of the pharmaceutical dosage form, the portion of azacitidine that is formulated for modified release is provided as enteric-coated minitablets or pellets. In some embodiments, the enteric coating, or the release modifying coating is pH sensitive.

In some embodiments at least a portion of the azacitidine is formulated for immediate release (e.g., release in the stomach). For example, about 5%, 10%, 15%, 20%, 25%, 30%, 33.3%, 35%, 40%, 45%, 50%, 55%, 60%, 65%, 70%, 75%, 80%, 85%, 90%, 95% or 100% of the azacitidine, by weight, may be formulated for immediate release. In some embodiments, about ⅓, or ⅔ of the azacitidine, by weight, may be formulated for immediate release. In some of such embodiments, the azacitidine formulated for immediate release is uncoated. In some embodiments the uncoated azacitidine is a powder. In some embodiments the uncoated azacitidine is a blend (e.g., powder blend). In some embodiments the uncoated azacitidine is in the form of granules. In some embodiments, the uncoated azacitidine is in the form of pellets. In some embodiments, the uncoated azacitidine is in the form of minitablets.

In some embodiments of the pharmaceutical dosage form, about 0% to about 60% of the azacitidine is provided as uncoated minitablets or pellets and about 100% to about 50% of the azacitidine is provided as modified release coated minitablets or pellets. In some embodiments of the pharmaceutical dosage form, about 0% to about 70% of the azacitidine is provided as uncoated minitablets or pellets and about 100% to about 30% of the azacitidine is provided as modified release coated minitablets or pellets. In some embodiments of the pharmaceutical dosage form, about 10% to about 65% of the azacitidine is provided as uncoated minitablets or pellets and about 90% to about 35% of the azacitidine is provided as modified release coated minitablets or pellets. In some embodiments of the pharmaceutical dosage form, about 20% to about 60% of the azacitidine is provided as uncoated minitablets or pellets and about 80% to about 40% of the azacitidine is provided as modified release coated minitablets or pellets. In some embodiments of the pharmaceutical dosage form, about 30% to about 65% of the azacitidine is provided as uncoated minitablets or pellets and about 70% to about 35% of the azacitidine is provided as modified release coated minitablets or pellets. In some embodiments of the pharmaceutical dosage form, about 30% to about 60% of the azacitidine is provided as uncoated minitablets or pellets and about 70% to about 40% of the azacitidine is provided as modified release coated minitablets or pellets. In some embodiments of the pharmaceutical dosage form, about 37% to about 60% of the azacitidine is provided as uncoated minitablets or pellets and about 40% to about 63% of the azacitidine is provided as modified release coated minitablets or pellets.

In some embodiments of the pharmaceutical dosage form, all of the azacitidine is formulated for modified release. In some embodiments of the pharmaceutical dosage form, none of the azacitidine is formulated for immediate release. In some embodiments of the pharmaceutical dosage form, substantially all of the azacitidine is formulated for modified release. In some of such embodiments, substantially all of the azacitidine is released outside of the stomach (e.g. in the intestine).

### Release Profile of Cedazuridine

While cedazuridine enhances bioavailability of azacitidine, the location of release of cedazuridine affects the enhancement of bioavailability of azacitidine, and it has been discovered that immediate release (release in stomach) of cedazuridine further helps the enhancement of bioavailability of azacitidine.

In one embodiment, a fixed dose combination, a pharmaceutical dosage form or a composition comprises cedazuridine or a pharmaceutically acceptable salt thereof, and azacitidine or a pharmaceutically acceptable salt thereof, wherein at least a portion of the cedazuridine is formulated for immediate release. In some embodiments, a portion of the cedazuridine is formulated for immediate release, and the remainder of the cedazuridine is formulated for modified release. For example, about 5%, 10%, 15%, 20%, 25%, 30%, 33.3%, 35%, 40%, 45%, 50%, 55%, 60%, 65%, 70%, 75%, 80%, 85%, 90%, 95% or 100% of the cedazuridine, by weight, may be formulated for immediate release. In some embodiments, about ⅓, or ⅔ of the cedazuridine, by weight, may be formulated for immediate release. In some embodiments the cedazuridine is formulated for immediate release, and none of the cedazuridine is formulated for modified release. In some embodiments, substantially all of the cedazuridine is formulated for immediate release. In some of such embodiments, the cedazuridine formulated for immediate release is uncoated. In some embodiments the uncoated cedazuridine is a powder. In some embodiments, the uncoated cedazuridine is a blend (e.g., powder blend). In some embodiments, the uncoated cedazuridine is in the form of granules. In some embodiments, the uncoated cedazuridine is in the form of pellets. In some embodiments the uncoated cedazuridine is in the form of minitablet. In some embodiments of the pharmaceutical dosage form, the portion of cedazuridine that is formulated for modified release is formulated for enteric release. In some embodiments of the pharmaceutical dosage form, the portion of cedazuridine that is formulated for immediate release is provided as uncoated minitablets or pellets. Such uncoated minitablets or pellets form cores which are optionally coated with release modifying coatings.

### Fixed Dose Combination (Immediate Release Cedazuridine+Modified Release Azacitidine)

In some embodiments, a fixed dose combination, a pharmaceutical dosage form or a composition comprises cedazuridine, or a pharmaceutically acceptable salt thereof, and azacitidine, or a pharmaceutically acceptable salt thereof, wherein at least a portion of the azacitidine is formulated for modified release and at least a portion of the cedazuridine is formulated for immediate release. In some embodiments, a fixed dose combination, a pharmaceutical dosage form or a composition comprises cedazuridine or a pharmaceutically acceptable salt thereof, and azacitidine or a pharmaceutically acceptable salt thereof, wherein at least a portion of the azacitidine is formulated for modified release and all or substantially all of the cedazuridine is formulated for immediate release. In some embodiments, a fixed dose combination, a pharmaceutical dosage form or a composition comprises cedazuridine, or a pharmaceutically acceptable salt thereof, and azacitidine, or a pharmaceutically acceptable salt thereof, wherein all or substantially all of the azacitidine is formulated for modified release and at least a portion of the cedazuridine is formulated for immediate release.

In some embodiments, a fixed dose combination, a pharmaceutical dosage form or a composition comprises cedazuridine, or a pharmaceutically acceptable salt thereof, and azacitidine, or a pharmaceutically acceptable salt thereof, wherein the azacitidine is formulated for modified release and the cedazuridine is formulated for immediate release. The azacitidine formulated for modified release may be coated. In some embodiments, the coated azacitidine is in the form of pellets or minitablets. In some of such embodiments, the cedazuridine formulated for immediate release is uncoated. In some embodiments the uncoated cedazuridine is a powder. In some embodiments, the uncoated cedazuridine is a blend (e.g., powder blend). In some embodiments, the uncoated cedazuridine is in the form of granules. In some embodiments, the uncoated cedazuridine is in the form of pellets. In some embodiments the uncoated cedazuridine is in the form of minitablets. Each of the cedazuridine and the azacitidine may be formulated according to the following description. For example, the fixed dose combination, pharmaceutical dosage form or composition includes cedazuridine in a form of uncoated minitablets or pellets described herein, and azacitidine in a form of minitablets or pellets coated for modified release described herein. The fixed dose combination may be provided in a form of capsule.

### Cedazuridine Formulation

The cedazuridine in the fixed dose combination, pharmaceutical dosage form or composition may be formulated such that the effect of enhancing azacitidine bioavailability is achieved. The cedazuridine in the fixed dose combination, pharmaceutical dosage form or composition may be in the form of a minitablet, tablet, powder, a blend, granules, or pellets, and may further include lactose monohydrate, a filler, a binder, a disintegrant, a glidant, and/or a lubricant. In some embodiments, the pharmaceutical dosage form of cedazuridine (i.e. cedazuridine minitablets, tablet, powder, blend, granules, or pellets) comprises about 10%-90% w/w, about 10%-80% w/w, about 10%-60% w/w, or about 10%-40% w/w of cedazuridine, about 50%-90% w/w of cedazuridine, about 60%-90% w/w of cedazuridine, about 50%-85% w/w of cedazuridine, about 60%-85% w/w of cedazuridine, about 70%-90% w/w of cedazuridine, about 70%-85% w/w of cedazuridine, about 75%-85% w/w of cedazuridine, about 75%-80% w/w of cedazuridine, about 80%-85% w/w of cedazuridine, about 80%-90% w/w of cedazuridine, or about 70%-80% w/w of cedazuridine, about 15%-40% w/w of cedazuridine, about 25%-40% w/w of cedazuridine, about 20%-40% w/w of cedazuridine, about 30%-40% w/w of cedazuridine, about 15%-35% w/w of cedazuridine, about 15%-30% w/w of cedazuridine, or about 20%-30% w/w of cedazuridine, wherein the percentage by weight is relative to the total weight of the uncoated cedazuridine minitablets, tablet, powder, blend, granules, or pellets.

In some embodiments, the cedazuridine is in the form of one or more tablets, minitablets or pellets. For example, the fixed dose combination, pharmaceutical dosage form or composition may comprise azacitidine or pharmaceutically acceptable salt thereof, and cedazuridine or pharmaceutically acceptable salt thereof, wherein the cedazuridine is in the form of one or more tablets, minitablets or pellets, and cedazuridine tablets, minitablets or pellets may comprise about 10%-40% w/w of cedazuridine, about 10%-30% w/w, about 15%-40% w/w, about 15%-30% w/w, or about 15%-25% w/w of cedazuridine wherein the percentage by weight is relative to the total weight of the uncoated cedazuridine tablets, minitablets or pellets. In some embodiments, the cedazuridine tablets, minitablets, or pellets further comprise about 40%-80% w/w of lactose monohydrate, about 0.5-10% w/w of hydroxypropyl methylcellulose (HPMC), about 1%-10% w/w of croscarmellose sodium, about 0.1%-3% w/w of silicon dioxide, and about 0.1%-3% w/w of magnesium stearate, wherein the percentage by weight is relative to the total weight of the uncoated cedazuridine tablets, minitablets, or pellets. In some embodiments, the cedazuridine pellet or minitablet comprise about 10%-30% w/w of cedazuridine, 40%-80% w/w of lactose monohydrate, about 0.5-10% w/w of hydroxypropyl methylcellulose (HPMC), about 1%-10% w/w of croscarmellose sodium, about 0.1%-3% w/w of silicon dioxide, and about 0.1%-3% w/w of magnesium stearate, wherein the percentage by weight is relative to the total weight of the uncoated cedazuridine tablets, minitablets, or pellets. In some embodiments, the cedazuridine tablets, minitablets, or pellets comprise about 20% w/w of cedazuridine, about 71.5% w/w of lactose monohydrate, about 2% w/w of hydroxypropyl methylcellulose (HPMC), about 5% w/w of croscarmellose sodium, about 1.0% w/w of silicon dioxide, and about 0.5% w/w of magnesium stearate, wherein the percentage by weight is relative to the total weight of the uncoated cedazuridine tablets, minitablets, or pellets.

In some embodiments, the cedazuridine is in the form of powder, blend, or granules. For example, the fixed dose combination, pharmaceutical dosage form or composition may comprise azacitidine or pharmaceutically acceptable salt thereof, and cedazuridine or pharmaceutically acceptable salt thereof, wherein the cedazuridine is in the form of powder or granules, and the cedazuridine powder or granules may comprise about 70%-90% w/w of cedazuridine, about 70%-85% w/w of cedazuridine, about 75%-90% w/w of cedazuridine, or about 75%-85% w/w of cedazuridine, wherein the percentage by weight is relative to the total weight of the uncoated cedazuridine powder or granules. The cedazuridine powder or granules may further comprise about 10%-20% w/w of lactose monohydrate, about 1%-10.5% w/w of croscarmellose sodium, about 0.1%-3% w/w of silicon dioxide, and about 0.1%-3% w/w of magnesium stearate, wherein the percentage by weight is relative to the total weight of the uncoated cedazuridine powder or granules. In some embodiments, the cedazuridine powder or granule comprises about 80% w/w of cedazuridine, 13.5% w/w of lactose monohydrate, about 5% w/w of croscarmellose sodium, about 1.0% w/w of silicon dioxide, and about 0.5% w/w of magnesium stearate, wherein the percentage by weight is relative to the total weight of the uncoated cedazuridine powder or granule.

### Azacitidine Formulation

The azacitidine in the fixed dose combination, pharmaceutical dosage form or composition may be formulated such that the azacitidine bioavailability is enhanced. In some embodiments of the fixed dose combination, pharmaceutical dosage form, or pharmaceutical composition, the azacitidine is provided as minitablets or pellets. Uncoated azacitidine minitablets or pellets form cores which are optionally coated with release modifying coatings. In some embodiments of the pharmaceutical dosage form, the portion of azacitidine that is formulated for modified release is provided as enteric-coated minitablets or pellets. In some embodiments, the enteric coating, or the release modifying coating is pH sensitive.

The azacitidine minitablets or pellets includes azacitidine and one or more pharmaceutically acceptable excipients, such as lactose monohydrate, a filler, a binder, a disintegrant, a glidant, and/or a lubricant. The azacitidine minitablets or pellets of azacitidine may comprise about 10%-70% w/w, about 10%-60% w/w, about 10%-50% w/w, about 10%-40% w/w, about 15%-40% w/w, about 20%-40% w/w, about 25%-40% w/w, about 30%-40% w/w, about 20%-60% w/w, about 25%-50% w/w, about 25%-45% w/w, about 30%-45% w/w, about 20%-60% w/w, about 20%-50% w/w, about 30%-50% w/w, or about 35%-45% w/w of azacitidine, wherein the percentage by weight is relative to the total weight of the uncoated minitablets or pellets. In some embodiments, the azacitidine minitablets or pellets comprises about 40% w/w of azacitidine, wherein the percentage by weight is relative to the total weight of the uncoated azacitidine minitablets or pellets.

In some embodiments the pharmaceutical dosage form comprises about 10%-60% w/w of lactose monohydrate, wherein the percentage by weight is relative to the total weight of the uncoated azacitidine minitablets or pellets. In some embodiments the pharmaceutical dosage form comprises about 10%-50% w/w, about 10%-40% w/w, about 10%-30% w/w, or about 10%-20% w/w, of lactose monohydrate, wherein the percentage by weight is relative to the total weight of the uncoated azacitidine minitablets or pellets. In some embodiments the pharmaceutical dosage form comprises about 10%-60% w/w of microcrystalline cellulose, wherein the percentage by weight is relative to the total weight of the uncoated azacitidine minitablets or pellets. In some embodiments the pharmaceutical dosage form comprises about 10%-50% w/w, about 20%-40% w/w, or about 20%-30% w/w of microcrystalline cellulose, wherein the percentage by weight is relative to the total weight of the uncoated azacitidine minitablets or pellets. In some embodiments the pharmaceutical dosage form comprises about 0.5-10% w/w of hydroxypropyl methylcellulose (HPMC), wherein the percentage by weight is relative to the total weight of the uncoated azacitidine minitablets or pellets. In some embodiments the pharmaceutical dosage form comprises about 0.5-8% w/w, about 0.5-6% w/w, about 0.5-4% w/w, or about 1-4% w/w of hydroxypropyl methylcellulose (HPMC), wherein the percentage by weight is relative to the total weight of the uncoated azacitidine minitablets or pellets. In some embodiments the pharmaceutical dosage form comprises about 1%-10% w/w of croscarmellose sodium, wherein the percentage by weight is relative to the total weight of the uncoated azacitidine minitablets or pellets. In some embodiments the pharmaceutical dosage form comprises about 1%-8% w/w, about 2%-8% w/w, or about 3%-6% w/w of croscarmellose sodium, wherein the percentage by weight is relative to the total weight of the uncoated azacitidine minitablets or pellets. In some embodiments the pharmaceutical dosage form comprises about 0.1%-3% w/w of silicon dioxide, wherein the percentage by weight is relative to the total weight of the uncoated azacitidine minitablets or pellets. In some embodiments the pharmaceutical dosage form comprises about 0.1%-2% w/w, or about 0.5%-2% w/w of silicon dioxide, wherein the percentage by weight is relative to the total weight of the uncoated azacitidine minitablets or pellets. In some embodiments the pharmaceutical dosage form comprises about 0.1%-3% w/w of magnesium stearate, wherein the percentage by weight is relative to the total weight of the uncoated azacitidine minitablets or pellets. In some embodiments the pharmaceutical dosage form comprises about 0.1%-2% w/w, or about 0.1%-1% of magnesium stearate, wherein the percentage by weight is relative to the total weight of the uncoated azacitidine minitablets or pellets.

In some embodiments, the azacitidine minitablets or pellets comprise about 20%-50% w/w of azacitidine, about 10%-60% w/w of lactose monohydrate, about 2%-50% w/w of microcrystalline cellulose, about 1%-10% w/w of croscarmellose sodium, about 0.5%-10% w/w of hydroxypropyl methylcellulose (HPMC), about 0.1%-4% w/w/ of silicon dioxide, and about 0.1%-3% w/w of magnesium stearate, wherein the percentage by weight is relative to the total weight of the uncoated azacitidine minitablets or pellets. In some embodiments, the azacitidine minitablets or pellets comprise comprises about 40% w/w of azacitidine, about 19.7% w/w of lactose monohydrate, about 30% w/w of microcrystalline cellulose, about 5% w/w of croscarmellose sodium, about 2% w/w of hydroxypropyl methylcellulose (HPMC), about 2.1% w/w of silicon dioxide, and about 1.2% w/w of magnesium stearate, wherein the percentage by weight is relative to the total weight of the uncoated azacitidine minitablets or pellets.

In some embodiments of the fixed dose combination, pharmaceutical dosage form or composition the azacitidine uncoated minitablets or pellets comprise one or more layers. For example, the azacitidine uncoated minitablets or pellets comprise one or more layers may comprise an intragranular layer and an extragranular layer.

In some embodiments the intragranular layer comprises about 70%-92% w/w of azacitidine minitablets or pellets, wherein the percentage by weight is relative to the total weight of the uncoated azacitidine minitablets or pellets. In some embodiments the intragranular layer comprises about 70%-95% w/w, about 75%-95% w/w, about 80%-95% w/w, about 85%-95% w/w, or about 90%-92% of azacitidine minitablets or pellets, wherein the percentage by weight is relative to the total weight of the uncoated azacitidine minitablets or pellets. In some embodiments the intragranular layer comprises about 90.5% w/w of azacitidine minitablets or pellets, wherein the percentage by weight is relative to the total weight of the uncoated azacitidine minitablets or pellets.

In some embodiments, in each uncoated minitablet or pellet, the intragranular layer comprises about 20%-50% w/w of azacitidine, wherein the percentage by weight is relative to the total weight of the uncoated azacitidine minitablets or pellets. In some embodiments, the intragranular layer comprises about 20%-60% w/w, about 25%-50% w/w, about 25%-45% w/w, about 30%-45% w/w, or about 35%-45% w/w of azacitidine, wherein the percentage by weight is relative to the total weight of the uncoated azacitidine minitablets or pellets. In some embodiments, the intragranular layer comprises about 10%-60% w/w of lactose monohydrate, wherein the percentage by weight is relative to the total weight of the uncoated azacitidine minitablets or pellets. In some embodiments, the intragranular layer comprises about 10%-40% w/w, about 10%-30% w/w, or about 15%-25% w/w, of lactose monohydrate, wherein the percentage by weight is relative to the total weight of the uncoated azacitidine minitablets or pellets. In some embodiments, the intragranular layer comprises about 2%-50% w/w of microcrystalline cellulose, wherein the percentage by weight is relative to the total weight of the uncoated azacitidine minitablets or pellets. In some embodiments, the intragranular layer comprises about 2%-40% w/w, about 5%-40% w/w, about 10%-40% w/w, about 15%-30% w/w or about 20%-30% w/w of microcrystalline cellulose, wherein the percentage by weight is relative to the total weight of the uncoated azacitidine minitablets or pellets. In some embodiments, the intragranular layer comprises about 1%-10% w/w of croscarmellose sodium, wherein the percentage by weight is relative to the total weight of the uncoated azacitidine minitablets or pellets. In some embodiments, the intragranular layer comprises about 1%-10% w/w, about 1%-8% w/w, about 1%-6% w/w, or about 1%-4% w/w of croscarmellose sodium, wherein the percentage by weight is relative to the total weight of the uncoated azacitidine minitablets or pellets. In some embodiments, the intragranular layer comprises about 0.5%-10% w/w of hydroxypropyl methylcellulose (HPMC), wherein the percentage by weight is relative to the total weight of the uncoated azacitidine minitablets or pellets. In some embodiments, the intragranular layer comprises about 0.5%-8% w/w, about 0.5%-6% w/w, about 0.5%-4% w/w, or about 1%-4% w/w of hydroxypropyl methylcellulose (HPMC), wherein the percentage by weight is relative to the total weight of the uncoated azacitidine minitablets or pellets. In some embodiments, the intragranular layer comprises about 0.1%-3% w/w of silicon dioxide, wherein the percentage by weight is relative to the total weight of the uncoated azacitidine minitablets or pellets. In some embodiments, the intragranular layer comprises about 0.1%-2% w/w, or about 0.1%-1% w/w of silicon dioxide, wherein the percentage by weight is relative to the total weight of the uncoated azacitidine minitablets or pellets. In some embodiments, the intragranular layer comprises about 0.1%-3% w/w of magnesium stearate, wherein the percentage by weight is relative to the total weight of the uncoated azacitidine minitablets or pellets. In some embodiments, the intragranular layer comprises about 0.1%-2% w/w, or about 0.1%-1% w/w of magnesium stearate, wherein the percentage by weight is relative to the total weight of the uncoated azacitidine minitablets or pellets.

In some embodiments, the intragranular layer comprises about 20%-50% w/w of azacitidine, about 10%-60% w/w of lactose monohydrate, about 2%-50% w/w of microcrystalline cellulose, about 1%-10% w/w of croscarmellose sodium, about 0.5%-10% w/w of hydroxypropyl methylcellulose (HPMC), about 0.1%-3% w/w/ of silicon dioxide, and about 0.1%-3% w/w of magnesium stearate, wherein the percentage by weight is relative to the total weight of the uncoated azacitidine minitablets or pellets. In some embodiments, the intragranular layer comprises about 40% w/w of azacitidine, about 19.7% w/w of lactose monohydrate, about 25% w/w of microcrystalline cellulose, about 2.5% w/w of croscarmellose sodium, about 2% w/w of HPMC, about 0.5% w/w/ of silicon dioxide, and about 0.8% w/w of magnesium stearate, wherein the percentage by weight is relative to the total weight of the uncoated azacitidine minitablets or pellets.

In some embodiments, the extragranular layer comprises about 1%-60% w/w of microcrystalline cellulose, wherein the percentage by weight is relative to the total weight of the uncoated azacitidine minitablets or pellets. In some embodiments, the extragranular layer comprises about 1%-50% w/w, about 1%-40% w/w, about 1%-30%, about 1%-20% w/w, or about 1%-10% w/w of microcrystalline cellulose, wherein the percentage by weight is relative to the total weight of the uncoated azacitidine minitablets or pellets. In some embodiments, the extragranular layer comprises about 1%-10% w/w of croscarmellose sodium, wherein the percentage by weight is relative to the total weight of the uncoated azacitidine minitablets or pellets. In some embodiments, the extragranular layer comprises about 1%-8% w/w, about 1%-6% w/w, about 1%-4% w/w or about 2%-4% w/w of croscarmellose sodium, wherein the percentage by weight is relative to the total weight of the uncoated azacitidine minitablets or pellets. In some embodiments, the extragranular layer comprises about 0.1%-3% w/w silicon dioxide, wherein the percentage by weight is relative to the total weight of the uncoated azacitidine minitablets or pellets. In some embodiments, the extragranular layer comprises about 0.1%-2% w/w silicon dioxide, wherein the percentage by weight is relative to the total weight of the uncoated azacitidine minitablets or pellets. In some embodiments, the extragranular layer comprises about 0.1%-3% w/w of magnesium stearate, wherein the percentage by weight is relative to the total weight of the uncoated azacitidine minitablets or pellets. In some embodiments, the extragranular layer comprises about 0.1%-2% w/w, or about 0.1%-1% w/w of magnesium stearate, wherein the percentage by weight is relative to the total weight of the uncoated azacitidine minitablets or pellets.

In some embodiments, the extragranular layer comprises about 1%-60% w/w of microcrystalline cellulose, about 1%-10% w/w of croscarmellose sodium, about 0.1%-3% w/w silicon dioxide, and about 0.1%-3% w/w of magnesium stearate, wherein the percentage by weight is relative to the total weight of the uncoated azacitidine minitablets or pellets. In some embodiments, the extragranular layer comprises about 5% w/w of microcrystalline cellulose, about 2.5% w/w of croscarmellose sodium, about 1.6% w/w silicon dioxide, and about 0.4% w/w of magnesium stearate, wherein the percentage by weight is relative to the total weight of the uncoated azacitidine minitablets or pellets.

In some embodiments of the fixed dose combination, pharmaceutical dosage form or composition the azacitidine uncoated minitablets or pellets comprise the extragranular layer and the intragranular layer, wherein the intragranular layer comprises about 20%-50% w/w of azacitidine, about 10%-60% w/w of lactose monohydrate, about 2%-50% w/w of microcrystalline cellulose, about 1%-10% w/w of croscarmellose sodium, about 0.5%-10% w/w of hydroxypropyl methylcellulose (HPMC), about 0.1%-3% w/w/ of silicon dioxide, and about 0.1%-3% w/w of magnesium stearate, and wherein the extragranular layer comprises about 1%-60% w/w of microcrystalline cellulose, about 1%-10% w/w of croscarmellose sodium, about 0.1%-3% w/w silicon dioxide, and about 0.1%-3% w/w of magnesium stearate, wherein the percentage by weight is relative to the total weight of the uncoated azacitidine minitablets or pellets. In some embodiments, the intragranular layer comprises about 40% w/w of azacitidine, about 19.7% w/w of lactose monohydrate, about 25% w/w of microcrystalline cellulose, about 2.5% w/w of croscarmellose sodium, about 2% w/w of HPMC, about 0.5% w/w/ of silicon dioxide, and about 0.8% w/w of magnesium stearate, and the extragranular layer comprises about 5% w/w of microcrystalline cellulose, about 2.5% w/w of croscarmellose sodium, about 1.6% w/w silicon dioxide, and about 0.4% w/w of magnesium stearate, wherein the percentage by weight is relative to the total weight of the uncoated azacitidine minitablets or pellets.

In some embodiments of the fixed dose combination, pharmaceutical dosage form or composition the azacitidine uncoated minitablets or pellets comprise the extragranular layer and the intragranular layer, wherein the intragranular layer comprises about 20%-50% w/w of azacitidine, about 10%-30% w/w of lactose monohydrate, about 2%-50% w/w of microcrystalline cellulose, about 1%-10% w/w of croscarmellose sodium, about 0.5%-10% w/w of hydroxypropyl methylcellulose (HPMC), about 0.1%-3% w/w/ of silicon dioxide, about 0.5%-5% w/w of binder (e.g. Kollidon VA64), and about 0.1%-3% w/w of magnesium stearate, and wherein the extragranular layer comprises about 1%-60% w/w of microcrystalline cellulose, about 1%-10% w/w of croscarmellose sodium, about 0.1%-3% w/w silicon dioxide, and about 0.1%-3% w/w of magnesium stearate, wherein the percentage by weight is relative to the total weight of the uncoated azacitidine minitablets or pellets. The extragranular layer comprises about 2%-10% w/w of microcrystalline cellulose, about 1%-5% w/w of croscarmellose sodium, about 0.1%-1% w/w of magnesium stearate, and about 1%-5% of binder (e.g. Kollidon VA64).

In some embodiments, the azacitidine minitablet or pellet is not separated into two layers, and comprises about 2%-10% w/w of azacitidine, about 50%-90% w/w of lactose monohydrate, about 2%-10% w/w of croscarmellose sodium, about 1-5% w/w of HPMC, about 0.1%-3% of colloidal silicon dioxide, and 0.1%-3% of magnesium stearate.

### Coating

In some embodiments of the fixed dose combination, pharmaceutical dosage form, or composition, the azacitidine that is formulated for modified release comprises azacitidine minitablets or pellets described herein, coated with one or more layers to enable enteric release. In some embodiments of the fixed dose combination, pharmaceutical dosage form, or composition, the azacitidine that is formulated for modified release comprises azacitidine uncoated minitablets or pellets described herein, coated with one or more layers of a seal coat. In some embodiments, the minitablets or pellets coated with the seal coat are further coated with a second seal coat, or an intermediate coat. In some embodiments, the minitablets or pellets coated with the seal coat and the second seal coat are further coated with an enteric coating, modified release coating, or delayed release coating, providing a 3-layer coating. By of example only, Examples 4 and 5 illustrates such an embodiment. In some embodiments of the fixed dose combination, the minitablets or pellets may be coated with a seal coat, and an enteric coating or modified release coating, providing a 2-layer coating.

In some embodiments, the seal coat or the second seal coat comprises ingredients to protect the core, such as hydroxypropyl methylcellulose (HPMC), polyethylene glycol (PEG), magnesium oxide or other ingredients suitable for a seal coat for a drug.

The enteric coating or modified release coating comprises ingredients to protect the core minitablets or pellets through the acidity of stomach, and allow release in the small intestine. In some embodiments, the delayed release coating comprises ethyl cellulose, or ethyl cellulose based polymer. For example, the delayed release coating comprises Surelease® E-7-19040. In some embodiments, the delayed release coating is insensitive to pH variations in the intestine. In some of such embodiments, the azacitidine is released outside the stomach.

In some embodiments of the fixed dose combination, pharmaceutical dosage form, or the composition, the enteric or delayed release coating comprises methacrylate based polymers. In some embodiments, the enteric or delayed release coating may include Eudragit® L30D 55, FS 30 D. FL 30 D-55, or L100. In some embodiments, the enteric or delayed release coating may include methyl methacrylate-methacrylic acid copolymers, hydroxypropyl methylcellulose acetate succinates, cellulose acetate phthalate, cellulose acetate succinate, polyvinyl acetate phthalate, or a copolymer thereof. The enteric or delayed release coating may further include triethyl citrate and/or talc. In some embodiments, the enteric delayed release coating is sensitive to pH variations in the intestine. In some of such embodiments, depending on the polymer present in the coating, the enteric or delayed release coating is chosen to provide release in the duodenum, the jejunum, the ileum, or the colon. In some of such embodiments, the azacitidine is released outside the stomach. By way of example only, Example 6 illustrates such an embodiment.

The coating for the azacitidine minitablets or pellets may be applied in an amount suitable for providing desired release profile. In some embodiments, the total coating for the azacitidine minitablets or pellets may have weight gains of about 5-25%, about 10-25%, about 15-25%, about 20-25%, about 5-20%, about 10-20%, about 15-20%, about 5-15%, about 10-15%, or about 5-10% compared to uncoated azacitidine minitablets or pellets. In some embodiments, the enteric or delayed release coating for the azacitidine minitablets or pellets may have weight gains of about 3-15%, about 3-12%, about 3-10%, about 4-10%, or about 4-7%, compared to uncoated azacitidine minitablets or pellets.

### EMBODIMENTS

In some or any of the preceding embodiments, provided herein is a fixed dose combination, pharmaceutical dosage form, or pharmaceutical composition comprising cedazuridine, or a pharmaceutically acceptable salt thereof, and azacitidine, or a pharmaceutically acceptable salt thereof, wherein the cedazuridine is formulated for immediate release and provided as powder, granules, pellets or minitablets; and all of the azacitidine is formulated for modified release and provided as enteric-coated minitablets or pellets. The fixed dose combination may be provided as a capsule.

In some of such embodiments, the azacitidine minitablets or pellets comprise an intragranular layer and an extragranular layer. The intragranular layer comprises about 20%-50% w/w of azacitidine, about 10%-60% w/w of lactose monohydrate, about 2%-50% w/w of microcrystalline cellulose, about 1%-10% w/w of croscarmellose sodium, about 0.5%-10% w/w of hydroxypropyl methylcellulose (HPMC), about 0.1%-3% w/w/ of silicon dioxide, and about 0.1%-3% w/w of magnesium stearate. The extragranular layer comprises about 1%-60% w/w of microcrystalline cellulose, about 1%-10% w/w of croscarmellose sodium, about 0.1%-3% w/w silicon dioxide, and about 0.1%-3% w/w of magnesium stearate, wherein the percentage by weight is relative to the total weight of the uncoated azacitidine minitablets or pellets. In some of such embodiments, the intragranular layer comprises about 40% w/w of azacitidine, about 19.7% w/w of lactose monohydrate, about 25% w/w of microcrystalline cellulose, about 2.5% w/w of croscarmellose sodium, about 2% w/w of HPMC, about 0.5% w/w/ of silicon dioxide, and about 0.8% w/w of magnesium stearate, and the extragranular layer comprises about 5% w/w of microcrystalline cellulose, about 2.5% w/w of croscarmellose sodium, about 1.6% w/w silicon dioxide, and about 0.4% w/w of magnesium stearate, wherein the percentage by weight is relative to the total weight of the uncoated azacitidine minitablets or pellets. In some of such embodiments, the azacitidine minitablets or pellets are coated with a seal coat and a enteric or modified release coat. The enteric or modified release coat comprises polymethacrylate based polymers, and is sensitive to pH in intestine. The azacitidine is substantially released outside the stomach.

In some of such embodiments, the cedazuridine is provided as pellets or minitablets and the cedazuridine pellets or minitablets comprise about 10%-30% w/w of cedazuridine, 40%-80% w/w of lactose monohydrate, about 0.5-10% w/w of hydroxypropyl methylcellulose (HPMC), about 1%-10% w/w of croscarmellose sodium, about 0.1%-3% w/w of silicon dioxide, and about 0.1%-3% w/w of magnesium stearate, wherein the percentage by weight is relative to the total weight of the uncoated cedazuridine tablets, minitablets, or pellets. In some of such embodiments, the cedazuridine tablets, minitablets, or pellets comprise about 20% w/w of cedazuridine, about 71.5% w/w of lactose monohydrate, about 2% w/w of hydroxypropyl methylcellulose (HPMC), about 5% w/w of croscarmellose sodium, about 1.0% w/w of silicon dioxide, and about 0.5% w/w of magnesium stearate, wherein the percentage by weight is relative to the total weight of the uncoated cedazuridine tablets, minitablets, or pellets.

In other of such embodiments, the cedazuridine is in the form of powder, and the cedazuridine powder may comprise about 70%-90% w/w of cedazuridine, about 10%-20% w/w of lactose monohydrate, about 1%-10.5% w/w of croscarmellose sodium, about 0.1%-3% w/w of silicon dioxide, and about 0.1%-3% w/w of magnesium stearate, wherein the percentage by weight is relative to the total weight of the uncoated cedazuridine powder or granules. In some embodiments, the cedazuridine powder or granule comprises about 80% w/w of cedazuridine, 13.5% w/w of lactose monohydrate, about 5% w/w of croscarmellose sodium, about 1.0% w/w of silicon dioxide, and about 0.5% w/w of magnesium stearate, wherein the percentage by weight is relative to the total weight of the uncoated cedazuridine powder or granule.

In some or any of the preceding embodiments, provided herein is a pharmaceutical dosage form comprising cedazuridine, or a pharmaceutically acceptable salt thereof, and azacitidine, or a pharmaceutically acceptable salt thereof, wherein the cedazuridine is formulated for immediate release; at least a portion of the azacitidine is formulated for immediate release and provided as uncoated minitablets or pellets, and the remainder of the azacitidine is formulated for modified release and provided as enteric-coated minitablets or pellets. In some of such embodiments, the azacitidine minitablets or pellets are uniform (i.e., do not comprise intragranular and extragranular layers). In some other of such embodiments, the azacitidine minitablets or pellets comprise an intragranular layer and an extragranular layer.

In some or any of the preceding embodiments, provided herein is a pharmaceutical dosage form comprising cedazuridine, or a pharmaceutically acceptable salt thereof, and azacitidine, or a pharmaceutically acceptable salt thereof, wherein the cedazuridine is formulated for immediate release; and all of the azacitidine is formulated for modified release and provided as delayed-release minitablets or pellets. In some of such embodiments, the azacitidine pellets comprise an intragranular layer and an extragranular layer. In some other of such embodiments, the azacitidine pellets are uniform (i.e., do not comprise intragranular and extragranular layers).

### Fixed Dose Combination—Tablet

In some embodiments, provided herein is a fixed dose combination (“FDC”) pharmaceutical dosage form comprising cedazuridine, or a pharmaceutically acceptable salt thereof, and azacitidine, or a pharmaceutically acceptable salt thereof, wherein the cedazuridine and azacitidine are formulated for immediate release. In some of such embodiments, the dosage form is a tablet. In some embodiments, the tablet is an uncoated tablet such as a FDC tablet of Example 12.

In some embodiments the FDC tablet comprises and intragranular layer and an extragranular layer. In some embodiments, the intragranular layer is about 50%-90% w/w of the FDC tablet, wherein the percentage by weight is relative to the total weight of the FDC tablet. In some embodiments, the extragranular layer is about 10%-50% w/w of the FDC tablet, wherein the percentage by weight is relative to the total weight of the FDC tablet. In some embodiments, the intragranular layer is about 80% w/w of the FDC tablet, and the extragranular layer is about 20% w/w of the FDC tablet, wherein the percentage by weight is relative to the total weight of the FDC tablet.

In some embodiments, the intragranular layer of the FDC tablet comprises about 1%-10% w/w of cedazuridine, wherein the percentage by weight is relative to the total weight of the FDC tablet. In some embodiments, the intragranular layer of the FDC tablet comprises about 1-10% w/w of azacitidine, wherein the percentage by weight is relative to the total weight of the FDC tablet. In some embodiments, the intragranular layer of the FDC tablet comprises about 10%-60% w/w of lactose monohydrate, wherein the percentage by weight is relative to the total weight of the FDC tablet. In some embodiments, the intragranular layer of the FDC tablet comprises about 10%-50% w/w of microcrystalline cellulose, wherein the percentage by weight is relative to the total weight of the FDC tablet. In some embodiments, the intragranular layer of the FDC tablet comprises about 1%-10% w/w of croscarmellose sodium, wherein the percentage by weight is relative to the total weight of the FDC tablet. In some embodiments, the intragranular layer of the FDC tablet comprises about 1%-10% w/w of HPMC, wherein the percentage by weight is relative to the total weight of the FDC tablet. In some embodiments, the intragranular layer of the FDC tablet comprises about 0.1%-5% w/w of silicon dioxide, wherein the percentage by weight is relative to the total weight of the FDC tablet. In some embodiments, the intragranular layer of the FDC tablet comprises about 0.1%-5% w/w of magnesium stearate, wherein the percentage by weight is relative to the total weight of the FDC tablet.

In some embodiments, the extragranular layer of the FDC tablet comprises about 5%-30% w/w of microcrystalline cellulose, wherein the percentage by weight is relative to the total weight of the FDC tablet. In some embodiments, the extragranular layer of the FDC tablet comprises about 1%-10% w/w of croscarmellose sodium, wherein the percentage by weight is relative to the total weight of the FDC tablet. In some embodiments, the extragranular layer of the FDC tablet comprises about 1%-5% w/w of silicon dioxide, wherein the percentage by weight is relative to the total weight of the FDC tablet. In some embodiments, the intragranular layer of the FDC tablet comprises about 0.1%-5% w/w of magnesium stearate, wherein the percentage by weight is relative to the total weight of the FDC tablet.

In some embodiments, provided herein is a fixed dose combination pharmaceutical dosage form comprising cedazuridine, or a pharmaceutically acceptable salt thereof, and azacitidine, or a pharmaceutically acceptable salt thereof, wherein the cedazuridine and azacitidine are formulated for immediate release, and the dosage form is further coated with an enteric coating. In some of such embodiments, the dosage form is a tablet. In some embodiments, the tablet is a coated tablet such as a coated FDC tablet of Example 1. In some embodiments, the tablet is coated with a seal coat, and then with an enteric coating.

### Fixed Dose Combination—Enteric Capsule

In some embodiments, provided herein is a fixed dose combination pharmaceutical dosage form comprising cedazuridine, or a pharmaceutically acceptable salt thereof, and azacitidine, or a pharmaceutically acceptable salt thereof, wherein the cedazuridine and azacitidine are formulated for immediate release, and the dosage form is a capsule. In some embodiments, the capsule may be coated to provide an enteric capsule.

In one embodiment, provided herein is a capsule comprising azacitidine wherein the capsule comprises an enteric capsule shell. In some embodiments, the capsule further comprises cedazuridine. In some of such embodiments, the azacitidine and/or the cedazuridine may be filled in the capsule with additional excipients. In some embodiments, the azacitidine and/or the cedazuridine filled in the capsule may be in the form of a powder, a blend, granules, pellets, minitablets, or combinations thereof.

In some embodiments, the capsule shell is an enteric capsule shell which comprises hydroxypropylmethyl cellulose acetate succinate (HPMCAS), and/or hydroxypropylmethyl cellulose (HPMC). In some embodiments, the capsule shell is a Capsugel® Vcaps® enteric capsule. Vcaps® enteric capsules are manufactured with pharmaceutical-grade cellulosic enteric derivatives (e.g., HPMCAS, HPMC). In some embodiments, the capsules further comprise gelling agents and water. In some embodiments, the capsules further comprise at least one basic compound or basic composition capable of neutralizing succinic acid groups on HPMCAS, including but not limited to basic hydroxide compounds such as potassium hydroxide (KOH), sodium hydroxide (NaOH), calcium hydroxide (Ca(OH)₂), or other basic compounds or compositions, for example, ammonium hydroxide, cationic polymers such as EUDRAGIT® E PO, and mixtures thereof. In some embodiments, such an enteric capsule shell allows for release of the azacitidine and/or cedazuridine substantially outside of the stomach. In some embodiments, such an enteric capsule shell allows for enteric release of azacitidine. In some embodiments, the enteric release is delayed release which is sensitive to pH variations in the intestine.

In some embodiments, the enteric capsule shell comprises a solid filled in the enteric capsule shell. In some embodiments, the solid filled in the enteric capsule shell comprises azacitidine and additional excipients. In some embodiments, the solid filled in the enteric capsule shell comprises azacitidine and additional excipients, and further comprises cedazuridine and additional excipients.

In some embodiments, the capsule comprises a buffer salt therein. In some embodiments, the buffer salt is selected from sodium phosphate (including monobasic sodium phosphate, dibasic sodium phosphate), potassium phosphate, 2-amino-2-hydroxymethyl-propane-1,3-diol (tris), sodium hydroxide, sodium citrate, sodium acetate, potassium acetate, citric acid and sodium or potassium citrate, amino acid salts, malic acid and sodium or potassium malate, tartaric acid and potassium or sodium tartrate, glutamic acid and sodium or potassium glutamate, and sodium carbonate.

In some embodiments, the capsule further comprises about 10%-50% w/w, about 10%-40% w/w, about 10%-30% w/w, or about 10%-20% w/w, of lactose monohydrate, wherein the percentage by weight is relative to the total weight of the solid filled in the capsule shell. In some embodiments, the capsule further comprises about 26.2% w/w of lactose monohydrate, wherein the percentage by weight is relative to the total weight of the solid filled in the capsule shell. In some embodiments, the capsule further comprises about 10%-50% w/w, about 10%-40% w/w, about 10%-30% w/w, or about 20%-30% w/w of microcrystalline cellulose, wherein the percentage by weight is relative to the total weight of the solid filled in the capsule shell. In some embodiments, the capsule further comprises about 25.7% w/w microcrystalline cellulose, wherein the percentage by weight is relative to the total weight of the solid filled in the capsule shell. In some embodiments, the capsule further comprises about 1%-25% w/w, about 1%-20% w/w, about 5%-20% w/w, about 5%-15% w/w, or about 5%-10% w/w of croscarmellose sodium, wherein the percentage by weight is relative to the total weight of the solid filled in the capsule shell. In some embodiments, the capsule further comprises about 8% w/w croscarmellose sodium, wherein the percentage by weight is relative to the total weight of the solid filled in the capsule shell. In some embodiments, the capsule further comprises about 1%-10% w/w, about 1%-8% w/w, about 1%-6% w/w, or about 1%-4% w/w of HPMC, wherein the percentage by weight is relative to the total weight of the solid filled in the capsule shell. In some embodiments, the capsule further comprises about 2% w/w HPMC, wherein the percentage by weight is relative to the total weight of the solid filled in the capsule shell. In some embodiments, the capsule further comprises about 1%-10% w/w, about 1%-8% w/w, about 1%-6% w/w, or about 1%-4% w/w of silicon dioxide, wherein the percentage by weight is relative to the total weight of the solid filled in the capsule shell. In some embodiments, the capsule further comprises about 2.1% w/w of silicon dioxide, wherein the percentage by weight is relative to the total weight of the solid filled in the capsule shell. In some embodiments, the capsule further comprises about 0.1-10% w/w, about 0.1-8% w/w, about 0.1-5% w/w, about 0.1-2% w/w, or about 0.1-1% w/w of magnesium stearate, wherein the percentage by weight is relative to the total weight of the solid filled in the capsule shell. In some embodiments, the capsule further comprises about 1% w/w of magnesium stearate, wherein the percentage by weight is relative to the total weight of the solid filled in the capsule shell.

In some embodiments, the capsule further comprises about 10%-50% w/w of lactose monohydrate, about 10%-50% w/w of microcrystalline cellulose, about 1%-20% w/w croscarmellose sodium, about 1%-10% w/w HPMC, about 1%-10% w/w silicon dioxide, and about 0.1%-5% w/w magnesium stearate, wherein the percentage by weight is relative to the total weight of the solid filled in the capsule shell. In some embodiments, the capsule further comprises about 26.2% w/w of lactose monohydrate, about 25.7% w/w of microcrystalline cellulose, about 8% w/w croscarmellose sodium, about 2% w/w HPMC, about 2.1% w/w silicon dioxide, and about 1% w/w magnesium stearate, wherein the percentage by weight is relative to the total weight of the solid filled in the capsule shell.

In some embodiments, the capsule comprises about 5%-40% w/w of azacitidine, wherein the percentage by weight is relative to the total weight of the solid filled in the capsule shell. In some embodiments, the capsule comprises about 5%-30% w/w, about 5%-25% w/w, about 5%-20% w/w, or about 5%-15% w/w of azacitidine, wherein the percentage by weight is relative to the total weight of the solid filled in the capsule shell. In some embodiments, the capsule comprises about 10% w/w of azacitidine, wherein the percentage by weight is relative to the total weight of the solid filled in the capsule shell.

In some embodiments, the capsule comprises about 5%-40% w/w of cedazuridine, wherein the percentage by weight is relative to the total weight of the solid filled in the capsule shell. In some embodiments, the capsule comprises about 5%-30% w/w, about 5%-25% w/w, about 5%-20% w/w, or about 5%-15% w/w of cedazuridine, wherein the percentage by weight is relative to the total weight of the solid filled in the capsule shell. In some embodiments, the capsule comprises about 10% w/w of cedazuridine, wherein the percentage by weight is relative to the total weight of the solid filled in the capsule shell.

In some embodiments, the capsule further comprises about 5%-25% w/w sodium phosphate, wherein the percentage by weight is relative to the total weight of the solid filled in the capsule shell. In some embodiments, the capsule further comprises about 1%-30% w/w, about 1%-25% w/w, about 1%-20% w/w, about 1%-10% w/w, about 5%-20% w/w, or about 5%-10% w/w sodium phosphate, wherein the percentage by weight is relative to the total weight of the solid filled in the capsule shell. In some embodiments, the capsule further comprises about 15% w/w sodium phosphate, wherein the percentage by weight is relative to the total weight of the solid filled in the capsule shell. In any of these embodiments, the sodium phosphate is dibasic anhydrous sodium phosphate.

In some embodiments, the capsule comprises about 2%-20% w/w cedazuridine, wherein the percentage by weight is relative to the total weight of the solid filled in the capsule shell. In some embodiments, the capsule comprises about 2%-20% azacitidine, wherein the percentage by weight is relative to the total weight of the solid filled in the capsule shell. In some embodiments, the capsule comprises about 1%-40% w/w lactose monohydrate, wherein the percentage by weight is relative to the total weight of the solid filled in the capsule shell. In some embodiments, the capsule comprises about 1%-40% w/w microcrystalline cellulose (e.g., Avicel PH102, Avicel PH200), wherein the percentage by weight is relative to the total weight of the solid filled in the capsule shell. In some embodiments, the capsule comprises about 5%-25% w/w sodium phosphate, wherein the percentage by weight is relative to the total weight of the solid filled in the capsule shell. In some of such embodiments, the sodium phosphate is dibasic sodium phosphate. In some of such embodiments, the sodium phosphate is anhydrous. In some other embodiments, the capsule does not comprise any sodium phosphate. In some embodiments, the capsule comprises about 1%-10% w/w croscarmellose sodium, wherein the percentage by weight is relative to the total weight of the solid filled in the capsule shell. In some embodiments, the capsule comprises about 0%-15% HPMC (also referred to herein as Hypromellose). In some embodiments, the capsule does not comprise any HPMC. In some embodiments, the capsule comprises about 1%-4% colloidal silicon dioxide, wherein the percentage by weight is relative to the total weight of the solid filled in the capsule shell. In some embodiments, the capsule comprises about 0.25%-4% magnesium stearate, wherein the percentage by weight is relative to the total weight of the solid filled in the capsule shell.

In some embodiments, the capsule comprises about 8-10% w/w cedazuridine, wherein the percentage by weight is relative to the total weight of the solid filled in the capsule shell. In some embodiments, the capsule comprises about 8%-10% azacitidine, wherein the percentage by weight is relative to the total weight of the solid filled in the capsule shell. In some embodiments, the capsule comprises about 25%-35% w/w lactose monohydrate, wherein the percentage by weight is relative to the total weight of the solid filled in the capsule shell. In some embodiments, the capsule comprises about 22%-26% w/w microcrystalline cellulose (e.g., Avicel PH102, Avicel PH200), wherein the percentage by weight is relative to the total weight of the solid filled in the capsule shell. In some embodiments, the capsule comprises about 15% w/w sodium phosphate, wherein the percentage by weight is relative to the total weight of the solid filled in the capsule shell. In some of such embodiments, the sodium phosphate is dibasic sodium phosphate, wherein the percentage by weight is relative to the total weight of the solid filled in the capsule shell. In some of such embodiments, the sodium phosphate is anhydrous. In some other embodiments, the capsule does not comprise any sodium phosphate. In some embodiments, the capsule comprises about 8% w/w croscarmellose sodium, wherein the percentage by weight is relative to the total weight of the solid filled in the capsule shell. In some embodiments, the capsule comprises about 2% HPMC (also referred to herein as Hypromellose), wherein the percentage by weight is relative to the total weight of the solid filled in the capsule shell. In some embodiments, the capsule does not comprise any HPMC, wherein the percentage by weight is relative to the total weight of the solid filled in the capsule shell. In some embodiments, the capsule comprises about 2%-2.5% colloidal silicon dioxide, wherein the percentage by weight is relative to the total weight of the solid filled in the capsule shell. In some embodiments, the capsule comprises about 1%-1.5% magnesium stearate, wherein the percentage by weight is relative to the total weight of the solid filled in the capsule shell.

### Dosage

In some embodiments, the pharmaceutical dosage form comprises about 5 to about 200 mg, about 10 to about 300 mg, about 20 to about 300 mg, about 20 to about 200 mg, about 20 to about 100 mg, about 5 to about 100 mg, about 5 to about 80 mg, about 20 to about 80 mg, about 5 to about 60 mg, about 20 to about 60 mg, about 5 to about 50 mg, about 30 to about 50 mg, about 10 to about 30 mg, about 30 to about 300 mg, about 40 to about 300 mg, about 50 to about 300 mg, about 50 to about 250 mg, about 50 to about 200 mg, about 60 to about 150 mg, about 70 to about 150 mg, about 70 to about 140 mg, about 80 to about 130 mg, about 90 to about 120 mg, about 90 to about 110 mg, about 10 to about 100 mg, about 10 to about 70 mg, about 20 to about 50 mg of cedazuridine, as an active pharmaceutical ingredient. In some embodiments, the pharmaceutical dosage form comprises about 10, 20, 30, 40, 50, 60, 70, 80, 90, 100, 110, 120, 130, 140, 150, 160, 170, 180, 190, or 200 mg of cedazuridine, as an active pharmaceutical ingredient.

In some embodiments, the pharmaceutical dosage form comprises about 5 to about 100 mg, about 20 to about 120 mg, about 40 to about 120 mg, about 60 to about 120 mg, about 80 to about 120 mg, about 100 to about 120 mg, about 5 to about 80 mg, about 10 to about 80 mg, about 10 to about 70 mg, about 20 to about 70 mg, about 20 to about 65 mg, about 25 to about 65 mg, about 25 to about 60 mg, about 30 to about 55 mg, about 30 to about 50 mg, about 35 to about 50 mg, or about 35 to about 45 mg of azacitidine, as an active pharmaceutical ingredient. In some embodiments, the pharmaceutical dosage form comprises about 4, 8, 12, 16, 20, 24, 28, 32, 36, 40, 44, 48, 52, 56, 60, 64, 68, 72, 76, 80, 84, 88, 92, 96, 100, 104, 108, 112, 116, or 120 mg of azacitidine, as an active pharmaceutical ingredient.

Any suitable combination of content of azacitidine and cedazuridine described herein, may be included in a fixed dose combination dosage form. For example, the fixed dose combination may include about 5 to about 200 mg of cedazuridine and about 20 to about 160 mg of azacitidine. In some of such embodiments, the fixed dose combination may include about 5 to about 100 mg of cedazuridine and about 20 to about 140 mg of azacitidine. In some of such embodiments, the fixed dose combination may include about 5 to about 80 mg of cedazuridine and about 40 to about 140 mg of azacitidine. In some of such embodiments, the fixed dose combination may include about 5 to about 60 mg of cedazuridine and about 60 to about 140 mg of azacitidine, about 5 to about 40 mg of cedazuridine and about 60 to about 120 mg of azacitidine, or about 10 to about 40 mg of cedazuridine and about 80 to about 120 mg of azacitidine. In some embodiments, the fixed dose combination may include about 20 mg of cedazuridine and about 80 mg of azacitidine. In some embodiments, the fixed dose combination may include about 20 mg of cedazuridine and about 84 mg of azacitidine. In some embodiments, the fixed dose combination may include about 20 mg of cedazuridine and about 88 mg of azacitidine. In some embodiments, the fixed dose combination may include about 20 mg of cedazuridine and about 92 mg of azacitidine. In some embodiments, the fixed dose combination may include about 20 mg of cedazuridine and about 96 mg of azacitidine. In some embodiments, the fixed dose combination may include about 20 mg of cedazuridine and about 100 mg of azacitidine. In some embodiments, the fixed dose combination may include about 20 mg of cedazuridine and about 104 mg of azacitidine. In some embodiments, the fixed dose combination may include about 20 mg of cedazuridine and about 108 mg of azacitidine. In some embodiments, the fixed dose combination may include about 20 mg of cedazuridine and about 112 mg of azacitidine. In some embodiments, the fixed dose combination may include about 20 mg of cedazuridine and about 120 mg of azacitidine. In some embodiments, the fixed dose combination may include about 100 mg of cedazuridine and about 40 mg of azacitidine. In some embodiments, the fixed dose combination may include about 20 mg of cedazuridine and about 40 mg of azacitidine. In some embodiments, the fixed dose combination may include about 40 mg of cedazuridine and about 100 mg of azacitidine. In some embodiments, the fixed dose combination may include about 40 mg of cedazuridine and about 80 mg of azacitidine, about 40 mg of cedazuridine and about 84 mg of azacitidine, about 40 mg of cedazuridine and about 88 mg of azacitidine, about 40 mg of cedazuridine and about 92 mg of azacitidine, or about 40 mg of cedazuridine and about 96 mg of azacitidine.

In some embodiments, the pharmaceutical dosage form or the fixed dose combination may include cedazuridine and azacitidine in the ratio of from about 1:20 to about 10:1, from about 1:10 to about 10:1, from about 1:8 to about 1:1, from about 1:8 to about 1:2, from about 1:8 to about 1:4, from about 1:6 to about 1:1, from about 1:6 to about 1:2, from about 1:6 to about 1:3, from about 1:6 to about 1:4, from about 1:6 to about 1:5, from about 1:5 to about 5:1, from about 1:5 to about 1:1, from about 1:5 to 1:2, from about 1:5 to 1:3, from about 1:5 to 1:4, from about 1:4 to about 4:1, from about 1:4 to about 1:1, from about 1:3 to about 3:1, from about 1:2 to about 2:1, from about 1:1 to about 1:5, from about 1:1 to about 1:4, from about 1:1 to about 1:3, or from about 1:1 to about 1:2 by weight of cedazuridine and azacitidine. In some embodiments, the fixed dose combination may include cedazuridine and azacitidine in the ratio of about 1:1, about 1:1.5, about 1:2, about 1:2.5, about 3:4, about 3:5, about 1:3, about 1:4, about 1:5, about 1:6, about 1:7, about 1:8, about 1.5:1, about 2:1, about 2.5:1, about 4:3, about 4:1, or about 5:3 by weight of cedazuridine and azacitidine.

The specific dose level of a composition of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of cedazuridine and/or azacitidine between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of cedazuridine and/or azacitidine administered per dose or per day. Daily dosage of cedazuridine and/or azacitidine may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compositions of the present application may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

### Treatment Methods and Uses

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of cancer. The therapeutically effective amount may vary depending on the subject, and disease or condition being treated, the weight and age of the subject, the severity of the disease or condition, and the manner of administering, which can readily be determined by one or ordinary skill in the art.

Provided herein is a method of treating cancer in a patient comprising administering any dosage form described herein to the patient in need thereof. In some embodiments, the cancer is selected from hematological cancers and solid cancers. In further embodiments, the hematological cancer is selected from myelodysplastic syndromes (MDS) and leukemia. In further embodiments, the solid cancer is selected from pancreatic cancer, ovarian cancer, prostate cancer, peritoneal cancer, non-small cell lung cancer, and breast cancer. In yet further embodiments, the leukemia is acute myeloid leukemia (AML) or chronic myeloid leukemia (CML). In some embodiments, the AML may be relapsed or refractory AML. In some embodiments, the AML may be before or after hematopoietic cell transplant. In some embodiments, the cancer is mucosal melanoma. In some embodiments, the leukemia is recurrent or refractory acute biphenotypic leukemia. In some embodiments, the AML patients may have achieved first complete remission (CR) or complete remission with incomplete blood count recovery (CRi) following intensive induction chemotherapy and are not able to complete intensive curative therapy.

In some embodiments, the cancer is selected from previously treated or untreated, de novo or secondary myelodysplastic syndromes (MDS), previously treated or untreated, de novo or secondary chronic myelomonocytic leukemia (CMML or CMMoL), previously treated or untreated, de novo or secondary chronic myelogenous leukemia (CML), and previously treated or untreated, de novo or secondary juvenile myelomonocytic leukemia (JMML). In some embodiments, the MDS may be with intermediate-1, intermediate-2, and high-risk International Prognostic Scoring System groups.

In some embodiments, the cancer is associated with refractory anemia (RA), refractory anemia with ringed sideroblasts (RARS), refractory anemia with excess blasts (RAEB), refractory anemia with excess blasts in transformation (RAEB-T).

In some embodiments, the cancer is selected from malignant peripheral nerve sheath tumors (MPNST), neurological cancer, breast cancer, hormone receptor positive tumor, head and neck cancer, primary central chondrosarcoma, myeloproliferative neoplasm (MPN), recurrent B-cell non-Hodgkin lymphoma, recurrent diffuse large B-cell lymphoma, recurrent Hodgkin lymphoma, relapsed/refractory multiple myeloma (RRMM), metastatic colorectal cancer (mCRC), metastatic castration-resistant prostate cancer (mCRPC), and lung cancer.

In some embodiments, the patient has moderate or severe hepatic impairment. In some embodiments, the patient has normal hepatic function.

In some embodiments, the patient has moderate or severe renal impairment. In some embodiments, the patient has normal renal function.

In some embodiments, the pharmaceutical dosage form described herein may be administered orally. In some embodiments, the pharmaceutical dosage form described herein may be administered once, twice, or three times a day. In some embodiments, the pharmaceutical dosage form described herein may be administered once a day, or once in 2, 3, 4, 5, 6, or 7 days. In some embodiments, the pharmaceutical dosage form described herein may be administered by cycle. For example, the pharmaceutical dosage form described herein may be administered for 2, 3, 4, 5, 6, 7, 8, 9, 10, 11, or 12 days in each cycle.

Also provided is a pharmaceutical dosage form described herein for use in treating cancer.

### Combination Therapy

In one embodiment, the compounds, pharmaceutical compositions, and/or dosage forms disclosed herein may be used in combination with one or more additional therapeutic agents that are being used and/or developed to treat cancers, T-cell lymphomas, such as bone marrow/stem cell transplant and/or CAR T cell therapies. In some embodiments, the pharmaceutical composition or a dosage form described herein may include one or more anti-cancer agents in addition to azacitidine and cedazuridine.

The different agents may be administered sequentially or simultaneously (in separate compositions or in the same composition). Useful classes of agents for combination therapy include, but are not limited to kinase inhibitors, CDA inhibitors, Anti-PD-1 monoclonal antibody.

In some embodiments, the one or more additional therapeutic agent may be tolinapant or venetoclax. In some embodiments, a dosage form may include tolinapant and/or venetoclax in addition to azacitidine and cedazuridine.

In some embodiments, the pharmaceutical composition including azacitidine and cedazuridine, may be administered in combination with other therapeutic agents. In some embodiments, the other therapeutic agents may be selected from the group consisting of: ADI-PEG 20, AMG-176, APG-115, APR-246, avelumab, bendamustine, bisantrene, brentuximab vedotin, capecitabine, CB-839, cisplatin, CS-01, cusatuzumab, cyclophosphamide, cytarabine, dasatinib, daunorubicin, DCLL9718S, decitabine, deferasirox, dexamethasone, durvalumab, eltrombopag, enasidenib, entinostat, entrectinib, enzalutamide, epacadostat, erythropoetin, etoposide, evorpacept, filgrastim, fludarabine phosphate, flumatinib, gemcitabine, gemtuzumab ozogamicin, gilteritinib, GM-CSF, GSK2879552, HMPL-523, homoharringtonine, IBI188, ibrutinib, idarubicin, itacitinib, ivosidenib, jaktinib, KPT-8602, LDE255, lenalidomide, lirilumab, LP-108, magrolimab, MAX-40279, mitoxantrone, mitoxantrone liposome, mocetinostat, moxifloxacin, nivolumab, olutasidenib, omacetaxine, oxaliplatin, paclitaxel, pembrolizumab, pevonedistat, pinometostat, pracinostat, quizartinib, revlimid, rigosertib, rituximab, romidepsin, RP7214, S64315, S65487, sabatolimab, seclidemstat, selumetinib, siremadlin, sirolimus, SL-401, SNDX-5613, sorafenib, talazoparib, tamibarotene, tolinapant, trastuzumab, tucidinostat, tyrosine kinase inhibitor, uproleselan, velcade, venetoclax, vincristine, visilizumab, vorinostat, and vosaroxin. In the treatment of any of conditions described herein, the pharmaceutical composition or dosage form described herein, may be administered in combination with non-chemotherapeutic treatments, such as iron, all-trans retinoic acid, allogeneic stem cell transplantation and/or platelet transfusions.

### Kits

Provided herein are kits that include a dosage form of the disclosure, and suitable packaging. In one embodiment, a kit further includes a label and/or instructions for use of the dosage form in the treatment of the indications, including the diseases or conditions, described herein.

### Dosage Forms & Excipients

The oral dosage forms described herein are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

Some embodiments described herein may be modified to include any additional suitable excipients. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

For preparing solid compositions such as tablets or capsules, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be formulated to provide a dosage form affording the advantage of prolonged action, or to protect from the acid conditions of the stomach. For example, the tablet or pill can include an inner dosage and an outer dosage component, the latter being in the form of an envelope or coat over the former. The two components can be separated by a seal coat layer that serves to resist disintegration in the stomach and permit the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

In some embodiments, the pellets and/or tablets described herein further comprise a film coating e.g., for limiting photolytic degradation. Suitable film coatings are selected by routine screening of commercially available preparations. In one embodiment, the film coating may be a polyvinylalcohol-based coating.

In some embodiments, the pharmaceutical compositions as described herein are formulated in a unit dosage or pharmaceutical dosage form. The term “unit dosage forms” or “pharmaceutical dosage forms” refers to physically discrete units suitable as unitary dosages for human patients and other mammals, each unit containing a predetermined quantity of active material calculated to produce the desired therapeutic effect, in association with a suitable pharmaceutical excipient and are provided, for example, as a capsule. The dosage forms are generally administered in a pharmaceutically effective amount. In some embodiments, the dosage forms are placed/stored in aluminum strip packing, moisture barrier blister foil, or bottle packs.

### EMBODIMENTS

Embodiment 1. A pharmaceutical dosage form comprising cedazuridine, or a pharmaceutically acceptable salt thereof, and azacitidine, or a pharmaceutically acceptable salt thereof, wherein at least a portion of the azacitidine is formulated for modified release.

Embodiment 2. The dosage form of embodiment 1, wherein at least a portion of the azacitidine is formulated for immediate release.

Embodiment 3. The dosage form of embodiment 1 or embodiment 2, wherein the cedazuridine is formulated for immediate release.

Embodiment 4. The dosage form of embodiment 3, wherein the cedazuridine is uncoated and in the form of a minitablet, a powder, a blend, granules, or pellets.

Embodiment 5. The dosage form of any preceding embodiment, comprising about 10%-40% w/w of cedazuridine, wherein the percentage by weight is relative to the total weight of the uncoated cedazuridine minitablet, powder, blend, granules, or pellets.

Embodiment 6. The dosage form of embodiment 5, comprising about 20% w/w of cedazuridine, wherein the percentage by weight is relative to the total weight of the uncoated cedazuridine minitablet, powder, blend, granules, or pellets.

Embodiment 7. The dosage form of any preceding embodiment, further comprising about 40%-80% w/w of lactose monohydrate, about 0.5-10% w/w of hydroxypropyl methylcellulose (HPMC), about 1%-10% w/w of croscarmellose sodium, about 0.1%-3% w/w of silicon dioxide, and about 0.1%-3% w/w of magnesium stearate, wherein the percentage by weight is relative to the total weight of the uncoated cedazuridine minitablet, powder, blend, granules, or pellets.

Embodiment 8. The dosage form of embodiment 7, further comprising about 71.5% w/w of lactose monohydrate, about 2% w/w of hydroxypropyl methylcellulose (HPMC), about 5% w/w of croscarmellose sodium, about 1% w/w of silicon dioxide, and about 0.5% w/w of magnesium stearate, wherein the percentage by weight is relative to the total weight of the uncoated cedazuridine minitablet, powder, blend, granules, or pellets.

Embodiment 9. The dosage form of any one of the preceding embodiments, wherein the cedazuridine is in the form of minitablet or pellet.

Embodiment 10. The dosage form of any one of the preceding embodiments, wherein the portion of the azacitidine that is formulated for modified release is formulated for enteric release.

Embodiment 11. The dosage form of any one of the preceding embodiments, wherein the portion of azacitidine that is formulated for immediate release is provided as uncoated minitablets.

Embodiment 12. The dosage form of embodiment 1, wherein the portion of azacitidine that is formulated for modified release is provided as enteric-coated minitablets.

Embodiment 13. The dosage form of any one of the preceding embodiments, comprising about 20%-60% w/w of azacitidine, wherein the percentage by weight is relative to the total weight of the uncoated azacitidine minitablets.

Embodiment 14. The dosage form of any one of the preceding embodiments, comprising about 40% w/w of azacitidine, wherein the percentage by weight is relative to the total weight of the uncoated azacitidine minitablets.

Embodiment 15. The dosage form of any one of the preceding embodiments, further comprising lactose monohydrate, a filler, a binder, a disintegrant, a glidant, and a lubricant.

Embodiment 16. The dosage form of any one of the preceding embodiments, further comprising about 10%-60% w/w of lactose monohydrate, about 10%-60% w/w of microcrystalline cellulose, about 0.5-10% w/w of hydroxypropyl methylcellulose (HPMC), about 1%-10% w/w of croscarmellose sodium, about 0.1%-3% w/w of silicon dioxide, and about 0.1%-3% w/w of magnesium stearate, wherein the percentage by weight is relative to the total weight of the uncoated azacitidine minitablets.

Embodiment 17. The dosage form of any one of the preceding embodiments, further comprising about 48.5% w/w of lactose monohydrate, about 25% w/w of microcrystalline cellulose, about 2% w/w of hydroxypropyl methylcellulose (HPMC) about 5% w/w of croscarmellose sodium, about 1% w/w of silicon dioxide, and about 0.5% w/w of magnesium stearate, wherein the percentage by weight is relative to the total weight of the uncoated azacitidine minitablets.

Embodiment 18. The dosage form of embodiment 11, wherein the azacitidine uncoated minitablets comprise an intragranular layer and an extragranular layer.

Embodiment 19. The dosage form of embodiment 18, wherein the intragranular layer comprises about 20%-50% w/w of azacitidine, about 10%-60% w/w of lactose monohydrate, about 2%-60% w/w of microcrystalline cellulose, about 1%-10% w/w of croscarmellose sodium, about 0.5%-10% w/w of hydroxypropyl methylcellulose (HPMC), about 0.1%-3% w/w/ of silicon dioxide, and about 0.1%-3% w/w of magnesium stearate, wherein the percentage by weight is relative to the total weight of the uncoated azacitidine minitablets.

Embodiment 20. The dosage form of embodiment 18 or 19, wherein the extragranular layer comprises about 1%-60% w/w of microcrystalline cellulose, about 1%-10% w/w of croscarmellose sodium, about 0.1%-3% w/w silicon dioxide, and about 0.1%-3% w/w of magnesium stearate, wherein the percentage by weight is relative to the total weight of the uncoated azacitidine minitablets.

Embodiment 21. The dosage form of any one of the preceding embodiments, wherein the portion of azacitidine that is formulated for enteric release comprises azacitidine uncoated minitablet coated with a seal coat.

Embodiment 22. The dosage form of embodiment 21, wherein the seal coat comprises hydroxypropyl methylcellulose (HPMC).

Embodiment 23. The dosage form of embodiment 21 or 22, wherein the minitablets coated with the seal coat are further coated with an enteric coating.

Embodiment 24. The dosage form of embodiment 23, wherein the enteric coating comprises ethyl cellulose.

Embodiment 25. The dosage form of embodiments 23 or 24, wherein the enteric coating is insensitive to pH variations in the intestine.

Embodiment 26. The dosage form of embodiment 23, wherein the enteric coating comprises polymethacrylate or copolymers thereof.

Embodiment 27. The dosage form of embodiment 23 or 26, wherein the enteric coating is sensitive to pH variations in the intestine.

Embodiment 28. The dosage form of any one of the preceding embodiments, wherein about 0% to about 60% of the azacitidine is provided as uncoated minitablets and about 100% to about 50% of the azacitidine is provided as modified release coated minitablets.

Embodiment 29. The dosage form of any one of embodiments 1-27, wherein about 37% to about 60% of the azacitidine is provided as uncoated minitablets and about 40% to about 63% of the azacitidine is provided as modified release coated minitablets.

Embodiment 30. The dosage form of any one of embodiments 1, 3-28, wherein all of the azacitidine is formulated for modified release.

Embodiment 31. The dosage form of any one of embodiments 1, 3-28, wherein substantially all of the azacitidine is released outside the stomach.

Embodiment 32. A pharmaceutical dosage form comprising cedazuridine, or a pharmaceutically acceptable salt thereof, and azacitidine, or a pharmaceutically acceptable salt thereof, wherein the cedazuridine is formulated for immediate release; at least a portion of the azacitidine is formulated for immediate release and provided as uncoated minitablets, and the remainder of the azacitidine is formulated for modified release and provided as enteric-coated minitablets.

Embodiment 33. A pharmaceutical dosage form comprising cedazuridine, or a pharmaceutically acceptable salt thereof, and azacitidine, or a pharmaceutically acceptable salt thereof, wherein the cedazuridine is formulated for immediate release; and the azacitidine is formulated for modified release and provided as enteric-coated minitablets.

Embodiment 34. The dosage form of embodiment 32 or embodiment 33, wherein the azacitidine minitablets comprise an intragranular layer and an extragranular layer.

Embodiment 35. A pharmaceutical dosage form comprising cedazuridine, or a pharmaceutically acceptable salt thereof, and azacitidine, or a pharmaceutically acceptable salt thereof, wherein the cedazuridine is formulated for immediate release; and the azacitidine is formulated for modified release and provided as delayed release minitablets.

Embodiment 36. The dosage form of embodiment 35, wherein the azacitidine minitablets comprise an intragranular layer and an extragranular layer.

Embodiment 37. The dosage form of any one of embodiments 32-36, wherein the cedazuridine is uncoated and in the form of a minitablet, a powder, a blend, granules, or pellets.

Embodiment 38. The dosage form of embodiment 37, comprising about 20% w/w of cedazuridine, wherein the percentage by weight is relative to the total weight of the uncoated cedazuridine minitablet, powder, blend, granules, or pellets.

Embodiment 39. The dosage form of any one embodiments 32-38, comprising about 40% w/w of azacitidine, wherein the percentage by weight is relative to the total weight of the uncoated azacitidine minitablets.

Embodiment 40. The dosage form according to any one of the preceding embodiments, wherein the dosage form is a capsule comprising one or more cedazuridine minitablets and one or more azacitidine minitablets.

Embodiment 41. A fixed dose combination pharmaceutical dosage form comprising cedazuridine, or a pharmaceutically acceptable salt thereof, and azacitidine, or a pharmaceutically acceptable salt thereof, wherein the cedazuridine and azacitidine are formulated for immediate release.

Embodiment 42. The dosage form of embodiment 41, wherein the dosage form is a tablet.

Embodiment 43. A fixed dose combination pharmaceutical dosage form comprising cedazuridine, or a pharmaceutically acceptable salt thereof, and azacitidine, or a pharmaceutically acceptable salt thereof, wherein the cedazuridine and azacitidine are formulated for immediate release, and the dosage form is further coated with an enteric coating.

Embodiment 44. The dosage form of embodiment 43, wherein the dosage form is a tablet.

Embodiment 45. A pharmaceutical dosage form comprising: cedazuridine or a pharmaceutically acceptable salt thereof; and azacitidine or a pharmaceutically acceptable salt thereof in a form of one or more azacitidine minitablets.

Embodiment 46. The pharmaceutical dosage form of embodiment 45, wherein each azacitidine minitablet is coated with an enteric coat.

Embodiment 47. The pharmaceutical dosage form of embodiment 46, wherein the enteric coat comprises polymethacrylate or copolymers thereof.

Embodiment 48. The pharmaceutical dosage form of embodiment 46 or 47, wherein the enteric coat is sensitive to pH variations in intestine.

Embodiment 49. The pharmaceutical dosage form of any one of embodiments 46-48, wherein each azacitidine minitablet further comprises a seal coat.

Embodiment 50, The pharmaceutical dosage form of embodiment 49, wherein the seal coat comprises hydroxypropylmethyl cellulose (HPMC).

Embodiment 51. The pharmaceutical dosage form of any one of embodiments 45-50, wherein each azacitidine minitablet comprises about 20%-50% w/w of azacitidine, wherein the percentage by weight is relative to the total weight of the uncoated azacitidine minitablets.

Embodiment 52. The pharmaceutical dosage form of any one of embodiments 45-51, wherein each of the azacitidine minitablets comprise an intragranular layer and an extragranular layer.

Embodiment 53. The pharmaceutical dosage form of embodiment 52, wherein the intragranular layer comprises about 20%-50% w/w of azacitidine, about 10%-60% w/w of lactose monohydrate, about 2%-60% w/w of microcrystalline cellulose, about 1%-10% w/w of croscarmellose sodium, about 0.5%-10% w/w of hydroxypropyl methylcellulose (HPMC), about 0.1%-3% w/w/ of silicon dioxide, and about 0.1%-3% w/w of magnesium stearate, wherein the percentage by weight is relative to the total weight of the uncoated azacitidine minitablets.

Embodiment 54. The pharmaceutical dosage form of embodiment 52 or 53, wherein the extragranular layer comprises about 1%-60% w/w of microcrystalline cellulose, about 1%-10% w/w of croscarmellose sodium, about 0.1%-3% w/w silicon dioxide, and about 0.1%-3% w/w of magnesium stearate, wherein the percentage by weight is relative to the total weight of the uncoated azacitidine minitablets.

Embodiment 55. The pharmaceutical dosage form of any one of embodiments 45-54, wherein each azacitidine minitablet comprises about 4 mg of azacitidine.

Embodiment 56. The pharmaceutical dosage form of any one of embodiments 45-65, comprising 8, 9 or 10 azacitidine minitablets.

Embodiment 57. The pharmaceutical dosage form of any one of embodiments 45-56, comprising about 32 mg, 36 mg, or 40 mug of azacitidine.

Embodiment 58. The pharmaceutical dosage form of any one of embodiments 45-57, wherein the cedazuridine is formulated for immediate release.

Embodiment 59. The pharmaceutical dosage form of any one of embodiments 45-58, wherein the cedazuridine is in a form of uncoated minitablet, pellet or powder.

Embodiment 60. The pharmaceutical dosage form of any one of embodiments 45-59, wherein the cedazuridine minitablet comprises about 10%-40% w/w of cedazuridine, wherein the percentage by weight is relative to the total weight of the uncoated cedazuridine minitablets.

Embodiment 61. The dosage form of embodiment 60, comprising about 20% w/w of cedazuridine, wherein the percentage by weight is relative to the total weight of the uncoated cedazuridine minitablet.

Embodiment 62. The dosage form of embodiment 60 or 61, further comprising about 40%-80% w/w of lactose monohydrate, about 0.5-10% w/w of hydroxypropyl methylcellulose (HPMC), about 1%-10% w/w of croscarmellose sodium, about 0.1%-3% w/w of silicon dioxide, and about 0.1%-3% w/w of magnesium stearate, wherein the percentage by weight is relative to the total weight of the uncoated cedazuridine minitablet, powder, blend, granules, or pellets.

Embodiment 63. The pharmaceutical dosage form of any one of embodiments 45-62, comprising about 100 mug of cedazuridine.

Embodiment 64. The pharmaceutical dosage form of any one of embodiments 51-63, further comprising venetoclax.

Embodiment 65. A capsule comprising the pharmaceutical dosage form of embodiments 32-40.

Embodiment 66. A capsule comprising azacitidine and cedazuridine wherein the capsule comprises an enteric capsule shell.

Embodiment 67. The capsule of embodiment 65 or 66, wherein the enteric capsule shell comprises hydroxypropylmethyl cellulose acetate succinate (HPMCAS), and/or hydroxypropylmethyl cellulose (HPMC).

Embodiment 68. The capsule of any one of embodiments 65-67, wherein the enteric capsule shell comprises a solid filled in the enteric capsule shell.

Embodiment 69. The capsule of any one of embodiments 65-68, further comprising a buffer salt.

Embodiment 70. The capsule of embodiment 69, wherein the buffer salt is selected from monobasic sodium phosphate, dibasic sodium phosphate, potassium phosphate, 2-Amino-2-hydroxymethyl-propane-1,3-diol (tris), sodium hydroxide, sodium citrate, sodium acetate, potassium acetate, citric acid and sodium or potassium citrate, amino acid salts, malic acid and sodium or potassium malate, tartaric acid and potassium or sodium tartarate, glutamic acid and sodium or potassium glutamate, and sodium carbonate.

Embodiment 71. The capsule of any one of embodiments 65-70, further comprising about 10%-50% w/w of lactose monohydrate, about 10%-50% w/w of microcrystalline cellulose, about 1%-20% w/w croscarmellose sodium, about 1%-10% w/w HPMC, about 1%-10% w/w silicon dioxide, and about 0.1%-5% w/w magnesium stearate.

Embodiment 72. The capsule of any one of embodiments 65-71, further comprising about 26.2% w/w of lactose monohydrate, about 25.7% w/w of microcrystalline cellulose, about 8% w/w croscarmellose sodium, about 2% w/w HPMC, about 2.1% w/w silicon dioxide, and about 1% w/w magnesium stearate.

Embodiment 73. The capsule of any one of embodiments 65-72, comprising about 5%-40% w/w of azacitidine.

Embodiment 74. The capsule of any one of embodiments 65-73, comprising about 10% w/w of azacitidine.

Embodiment 75. The capsule of any one of embodiments 66-74, comprising about 5%-40% % w/w of cedazuridine.

Embodiment 76. The capsule of any one of embodiments 66-75, comprising about 10% % w/w of cedazuridine.

Embodiment 77. The capsule of any one of embodiments 65-76, further comprising about 5%-25% w/w sodium phosphate.

Embodiment 78. The capsule of any one of embodiments 65-77, further comprising about 15% w/w sodium phosphate.

Embodiment 79. A method of treating cancer in a patient comprising administering the dosage form of any one of embodiments 1-64 or the capsule of any one of embodiments 65-78 to the patient in need thereof.

Embodiment 80. The method of embodiment 79, wherein the cancer is leukemia.

Embodiment 81. The method of embodiment 79, wherein the cancer is selected from previously treated or untreated, de novo or secondary myelodysplastic syndromes (MDS), previously treated or untreated, de novo or secondary chronic myelomonocytic leukemia (CMML), acute myeloid leukemia (AML) and chronic myeloid leukemia (CML), malignant peripheral nerve sheath tumors (MPNST), neurological cancer, breast cancer, hormone receptor positive tumor, head and neck cancer, primary central chondrosarcoma, myeloproliferative neoplasm (MPN), recurrent B-cell non-Hodgkin lymphoma, recurrent diffuse large B-cell lymphoma, recurrent Hodgkin lymphoma, relapsed/refractory multiple myeloma (RRMM), metastatic colorectal cancer (mCRC), metastatic castration-resistant prostate cancer (mCRPC), and lung cancer.

Embodiment 82. The method of any one of embodiments 79-81, wherein the cancer is selected from previously treated or untreated, de novo or secondary myelodysplastic syndromes (MDS), previously treated or untreated, de novo or secondary chronic myelomonocytic leukemia (CMML), acute myeloid leukemia (AML) chronic myeloid leukemia (CML).

Embodiment 83. The method of any one of embodiments 79-82, wherein the cancer is associated with refractory anemia, refractory anemia with ringed sideroblasts, or refractory anemia with excess blasts.

Embodiment 84. The method of any one of embodiments 79-83, further comprising another therapeutic agent.

Embodiment 85. The method of embodiment 84, wherein the another therapeutic agent is one or more selected from the list consisting of: ADI-PEG 20, AMG-176, APG-115, APR-246, avelumab, bendamustine, bisantrene, brentuximab vedotin, capecitabine, CB-839, cisplatin, CS-01, cusatuzumab, cyclophosphamide, cytarabine, dasatinib, daunorubicin, DCLL9718S, decitabine, deferasirox, dexamethasone, durvalumab, eltrombopag, enasidenib, entinostat, entrectinib, enzalutamide, epacadostat, erythropoetin, etoposide, evorpacept, filgrastim, fludarabine phosphate, flumatinib, gemcitabine, gemtuzumab ozogamicin, gilteritinib, GM-CSF, GSK2879552, HMPL-523, homoharringtonine, IBI188, ibrutinib, idarubicin, itacitinib, ivosidenib, jaktinib, KPT-8602, LDE255, lenalidomide, lirilumab, LP-108, magrolimab, MAX-40279, mitoxantrone, mitoxantrone liposome, mocetinostat, moxifloxacin, nivolumab, olutasidenib, omacetaxine, oxaliplatin, paclitaxel, pembrolizumab, pevonedistat, pinometostat, pracinostat, quizartinib, revlimid, rigosertib, rituximab, romidepsin, RP7214, S64315, S65487, sabatolimab, seclidemstat, selumetinib, siremadlin, sirolimus, SL-401, SNDX-5613, sorafenib, talazoparib, tamibarotene, tolinapant, trastuzumab, tucidinostat, tyrosine kinase inhibitor, uproleselan, velcade, venetoclax, vincristine, visilizumab, vorinostat, and vosaroxin.

Embodiment 86. The dosage form of any one of embodiments 1-64 or the capsule of any one of embodiments 65-78 for use in the treatment of cancer.

Embodiment 87. The dosage form of any one of embodiments 1-64 or the capsule of any one of embodiments 65-78 for use in manufacturing a medicament for the treatment of cancer.

Embodiment 88. The dosage form of embodiment 87, wherein the cancer is leukemia.

Embodiment 89. The dosage form of embodiment 87, wherein the cancer is selected from previously treated or untreated, de novo or secondary myelodysplastic syndromes (MDS), previously treated or untreated, de novo or secondary chronic myelomonocytic leukemia (CMML), acute myeloid leukemia (AML) and chronic myeloid leukemia (CML), malignant peripheral nerve sheath tumors (MPNST), neurological cancer, breast cancer, hormone receptor positive tumor, head and neck cancer, primary central chondrosarcoma, myeloproliferative neoplasm (MPN), recurrent B-cell non-Hodgkin lymphoma, recurrent diffuse large B-cell lymphoma, recurrent Hodgkin lymphoma, relapsed/refractory multiple myeloma (RRMM), metastatic colorectal cancer (mCRC), metastatic castration-resistant prostate cancer (mCRPC), and lung cancer.

Embodiment 90. The dosage form of any one of embodiments 86-89, wherein the cancer is selected from previously treated or untreated, de novo or secondary myelodysplastic syndromes (MDS), previously treated or untreated, de novo or secondary chronic myelomonocytic leukemia (CMML), acute myeloid leukemia (AML) chronic myeloid leukemia (CML).

Embodiment 91. The dosage form of any one of embodiments 86-90, wherein the cancer is associated with refractory anemia, refractory anemia with ringed sideroblasts, or refractory anemia with excess blasts.

Embodiment 92. Use of the dosage form of any one of embodiments 1-64 or the capsule of any one of embodiments 65-78 for the treatment of cancer.

Embodiment 93. Use of the dosage form of any one of embodiments 1-64 or the capsule of any one of embodiments 65-78 for manufacturing of a medicament for the treatment of cancer.

Embodiment 94. The use of embodiment 92 or 93, wherein the cancer is leukemia.

Embodiment 95. The use of embodiment 92 or 93, wherein the cancer is selected from previously treated or untreated, de novo or secondary myelodysplastic syndromes (MDS), previously treated or untreated, de novo or secondary chronic myelomonocytic leukemia (CMML), acute myeloid leukemia (AML) and chronic myeloid leukemia (CML), malignant peripheral nerve sheath tumors (MPNST), neurological cancer, breast cancer, hormone receptor positive tumor, head and neck cancer, primary central chondrosarcoma, myeloproliferative neoplasm (MPN), recurrent B-cell non-Hodgkin lymphoma, recurrent diffuse large B-cell lymphoma, recurrent Hodgkin lymphoma, relapsed/refractory multiple myeloma (RRMM), metastatic colorectal cancer (mCRC), metastatic castration-resistant prostate cancer (mCRPC), and lung cancer.

Embodiment 96. The use of any one of embodiments 92-95, wherein the cancer is selected from previously treated or untreated, de novo or secondary myelodysplastic syndromes (MDS), and previously treated or untreated, de novo or secondary chronic myelomonocytic leukemia (CMML).

Embodiment 97. The use of any one of embodiments 92-96 wherein the cancer is associated with refractory anemia, refractory anemia with ringed sideroblasts, or refractory anemia with excess blasts.

Embodiment 98. A combination for treating cancer, wherein the combination comprises a cedazuridine or a pharmaceutically acceptable salt thereof; and azacitidine or a pharmaceutically acceptable salt thereof, wherein the cedazuridine is formulated for immediate release; and the azacitidine is formulated for modified release and provided as delayed release minitablets.

Embodiment 99. A capsule comprising azacitidine wherein the capsule comprises an enteric capsule shell.

Embodiment 100. The capsule of embodiment 99, further comprising cedazuridine.

Embodiment 101. The capsule of embodiment 99 or 100, wherein the enteric capsule shell comprises hydroxypropylmethyl cellulose acetate succinate (HPMCAS), and/or hydroxypropylmethyl cellulose (HPMC).

Embodiment 102. The capsule of embodiments 99 to 101, wherein the enteric capsule shell comprises a solid filled in the enteric capsule shell.

Embodiment 103. The capsule of embodiments 99 to 102, further comprising a buffer salt.

Embodiment 104. The capsule of embodiment 103, wherein the buffer salt is selected from monobasic sodium phosphate, dibasic sodium phosphate, potassium phosphate, 2-Amino-2-hydroxymethyl-propane-1,3-diol (tris), sodium hydroxide, sodium citrate, sodium acetate, potassium acetate, citric acid and sodium or potassium citrate, amino acid salts, malic acid and sodium or potassium malate, tartaric acid and potassium or sodium tartarate, glutamic acid and sodium or potassium glutamate, and sodium carbonate.

Embodiment 105. The capsule of embodiments 99 to 104, further comprising about 10%-50% w/w of lactose monohydrate, about 10%-50% w/w of microcrystalline cellulose, about 1%-20% w/w croscarmellose sodium, about 1%-10% w/w HPMC, about 1%-10% w/w silicon dioxide, and about 0.1%-5% w/w magnesium stearate.

Embodiment 106. The capsule of embodiments 99 to 105, further comprising about 26.2% w/w of lactose monohydrate, about 25.7% w/w of microcrystalline cellulose, about 8% w/w croscarmellose sodium, about 2% w/w HPMC, about 2.1% w/w silicon dioxide, and about 1% w/w magnesium stearate.

Embodiment 107. The capsule of embodiments 99 to 106, comprising about 5%-40% w/w of azacitidine.

Embodiment 108. The capsule of embodiments 99 to 107, comprising about 10% w/w of azacitidine.

Embodiment 109. The capsule of embodiments 100 to 108, comprising about 5%-40% % w/w of cedazuridine.

Embodiment 110. The capsule of embodiments 100 to 109, comprising about 10% w/w of cedazuridine.

Embodiment 111. The capsule of embodiments 99 to 110, further comprising about 5%-25% w/w sodium phosphate.

Embodiment 112. The capsule of embodiments 99 to 111, further comprising about 15% w/w sodium phosphate.

Embodiment 113. A method of treating cancer in a patient comprising administering the capsule of any one of embodiments 99 to 113 to the patient in need thereof.

Embodiment 114. The method of embodiment 113, wherein the cancer is selected from previously treated or untreated, de novo or secondary myelodysplastic syndromes (MDS), and previously treated or untreated, de novo or secondary chronic myelomonocytic leukemia (CMML).

Embodiment 115. The method of embodiment 113 or 114, wherein the cancer is associated with refractory anemia, refractory anemia with ringed sideroblasts, or refractory anemia with excess blasts.

Embodiment 116. The capsule of embodiments 99 to 113 for use in the treatment of cancer.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Coated FDC Cedazuridine and Azacitidine Tablets

Both azacitidine and cedazuridine were formulated for modified release. 20 mg azacitidine and cedazuridine fixed dose combination (FDC) oval tablets with core tablet weight 500 mg (Table 1) were seal coated first followed by Surelease®: HPMC pore former at ratio 70/30 as a functional coat for 12% and 15% coating weight gain. The compositions of seal and Surelease® coat are in Tables 1-2 and 1-3, respectively. PK studies were performed on both 12% and 15% weight gain modified release tablets.

TABLE 1-1
Composition of oval FDC tablets containing
20 mg cedazuridine and 20 mg azacitidine
Core Tablet Composition

| | Azacitidine 20 mg/Cedazuridine 20 mg | |
|---|---|---|
| Ingredients | mg/tab | % |

Intragranular

| | | |
|---|---|---|
| Azacitidine | 20.0 | 4.0 |
| Cedazuridine | 20.0 | 4.0 |
| Lactose monohydrate | 206.6 | 41.3 |
| (FlowLac ® 90) | | |
| MCC (Avicel ® PH-102) | 128.4 | 25.7 |
| Croscarmellose Sodium | 12.5 | 2.5 |
| (Ac-di-sol ® SD-711) | | |
| Hypromellose E5 2910 | 10.0 | 2.0 |
| Colloidal Silicon Dioxide | 2.7 | 0.54 |
| Magnesium Stearate | 4.0 | 0.8 |
| (Hyqual ®, Vegetal) | | |
| Intragranular Total | 404.2 | 80.8 |

Extragranular

| | | |
|---|---|---|
| MCC (Avicel ® PH-200) | 74.0 | 14.8 |
| Croscarmellose Sodium | 12.5 | 2.5 |
| (Ac-di-sol ® SD-711) | | |
| Colloidal Silicon Dioxide | 8.0 | 1.6 |
| Magnesium Stearate. | 1.3 | 0.26 |
| (Hyqual ®, Vegetal) | | |
| Extragranular Total | 95.8 | 19.2 |
| Core tablet total, Oval | 500.0 | 100.0 |

TABLE 1-2
Seal Coat composition (200 mL preparation, 10% solids)

| Material | Theoretical amount (g) |
|---|---|
| Opadry ® complete film coating | 20 |
| system YS-1-1-19025-A-Clear | |
| Water | 180 |

TABLE 1-3
70:30 Surelease ® composition (2000 mL preparation, 10% solids)

| | Theoretical amount |
|---|---|
| Material | (g) |
| Opadry ® complete film coating system | 63 |
| YS-1-1-19025-A-Clear (Pore | |
| Former) | |
| Surelease ®* | 588 |
| Water | 1449 |

*Surelease dispersion at 25% solids

### Manufacturing Process

Azacitidine, cedazuridine, and all intragranular excipients were dispensed into individual containers and screened by hand through 30 mesh. Sieved azacitidine, cedazuridine, and intragranular excipients except for magnesium stearate were added into a Turbula T2 blender with suitable capacity and mixed at 25 rpm for 15 minutes. Intragranular magnesium stearate was then sieved and added to the blend, and the lubrication blending was performed in a Turbula T2 Mixer Blender for 3 minutes at 25 rpm.

The intragranular blend was then roller compacted into ribbons and ribbons were milled into granules. The milled granules were blended with screened extragranular excipients to make final blend. The final blend was compressed into tablets using oval tooling in a Ronchi single eccentric tablet press. The target weight of the tablets 500 mg±10%.

The uncoated tablets were seal coated at 1% weight gain followed by functional coating of Surelease to modulate the dissolution profile. HPMC from Coloron (Opadry® complete coating system YS-1-19025-A-Clear) was used both for seal coat and as a pore former in an ethyl cellulose dispersion (Surelease®) for a functional coat. The seal coated tablets were coated at 12% and 15% weight gain with Surelease®: Pore Former. Coating was performed in a pan coater.

### Dissolution Profile

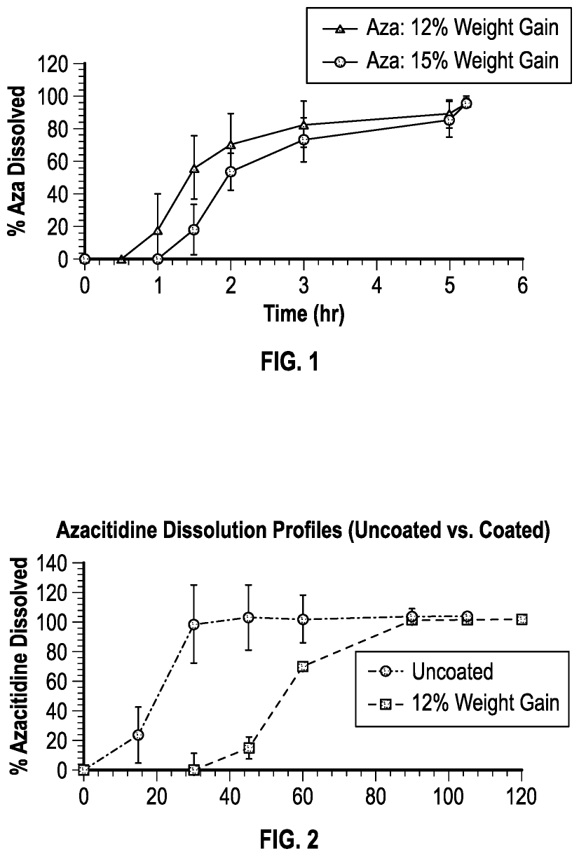

The coated FDC tablets (with 12% and 15% weight gain) were tested for dissolution in 500 mL, pH 6.8, 50 mM sodium phosphate buffer at 37° C. bath temperature with an agitation speed of 75 rpm in a basket. The drug release data was collected at 30, 60, 90, 120, 180, 300 mins and infinity at 315 mins with 250 rpm speed. The drug release profile for azacitidine is shown in FIG. 1.

### Example 2: Uncoated Cedazuridine 20 mg Tablets & Coated/Uncoated Azacitidine 5 mg Minitablets

### Manufacturing Process

Azacitidine and cedazuridine were screened through 30 mesh and pre-blended with all excipients listed in Tables 2-1, 2-2 except magnesium stearate. Magnesium stearate was then sieved and added to the blend, and blending was performed. The final blend was compressed into 0.25 inch round minitablets by direct compression. The target weight of the minitablets was 100 mg±7.5 mg.

TABLE 2-1
Cedazuridine 20 mg uncoated tablet

| Ingredient | mg | % |
|---|---|---|
| Cedazuridine | 20.0 | 20.0 |
| Lactose monohydrate (FlowLac 90) | 71.5 | 71.5 |
| Croscarmellose Sodium (Ac-di-sol SD-711) | 5.0 | 5.0 |
| Hypromellose E5 2910 | 2.0 | 2.0 |
| Colloidal silicon Dioxide | 1.0 | 1.0 |
| Magnesium Stearate (Hyqual, Vegetal) | 0.5 | 0.5 |
| Total | 100 | 100 |

TABLE 2-2
Azacitidine 5 mg minitablets

| Ingredient | mg | % |
|---|---|---|
| Azacitidine | 5.0 | 5.0 |
| Lactose monohydrate (FlowLac 90) | 86.0 | 86.0 |
| Croscarmellose Sodium (Ac-di-sol SD-711) | 5.0 | 5.0 |
| Hypromellose E5 2910 | 2.0 | 2.0 |
| Colloidal silicon Dioxide | 1.0 | 1.0 |
| Magnesium Stearate (Hyqual, Vegetal) | 1.0 | 1.0 |
| Total | 100 | 100 |

A portion of the azacitidine minitablets were seal coated first at 2% wt. gain followed by a functional coat of Surelease® with pore former at a ratio of 70:30. Opadry complete coating system from Colorcon, YS-1-19025-A-Clear was used both for a seal coat and as a pore former in an ethyl cellulose dispersion (Surelease) for a functional coat. The seal-coated azacitidine minitablets were further coated with enteric coat at 12% weight gain with Surelease/HPMC Pore former. The coating was performed in a pan coater. The composition of the seal coat and enteric coat is listed in Tables 2-3 and 2-4.

TABLE 2-3
Seal coating composition for azacitidine minitablets

| Ingredient | % |
|---|---|
| Opadry ® Clear, YS-1-19025-A (HPMC based) | 10 |
| Milli-Q water | 90 |

TABLE 2-4
Enteric coating (pH insensitive) for azacitidine minitablets

| Ingredient | % |
|---|---|
| Opadry ® Clear, YS-1-19025-A (HPMC based) | 3 |
| Surelease ® E-7-19040 (Ethyl cellulose based) | 7 |
| Milli-Q water | 90 |

### Dissolution Profile for Azacitidine

Both coated and uncoated azacitidine minitablets were tested for dissolution testing. Dissolution data was collected by placing either four (4) coated or uncoated azacitidine minitablets (5 mg) in size 0, HPMC capsules (Vcap plus). Dissolution testing was carried out in 500 mL, pH 6.8, 50 mM sodium phosphate buffer at 37° C. bath temperature with an agitation speed of 75 rpm in a basket. The drug release data was collected at 15, 30, 45, 60, 90, 105, and 120 mins (deemed infinity) with 250 rpm speed. Release data for azacitidine is in FIG. 2.

As shown in FIG. 2, the uncoated 4 azacitidine minitablets (20 mg total) showed immediate release with >85% release in 30 minutes, whereas coated azacitidine minitablets had a lag time of 30 minutes followed by complete release at 90 minutes.

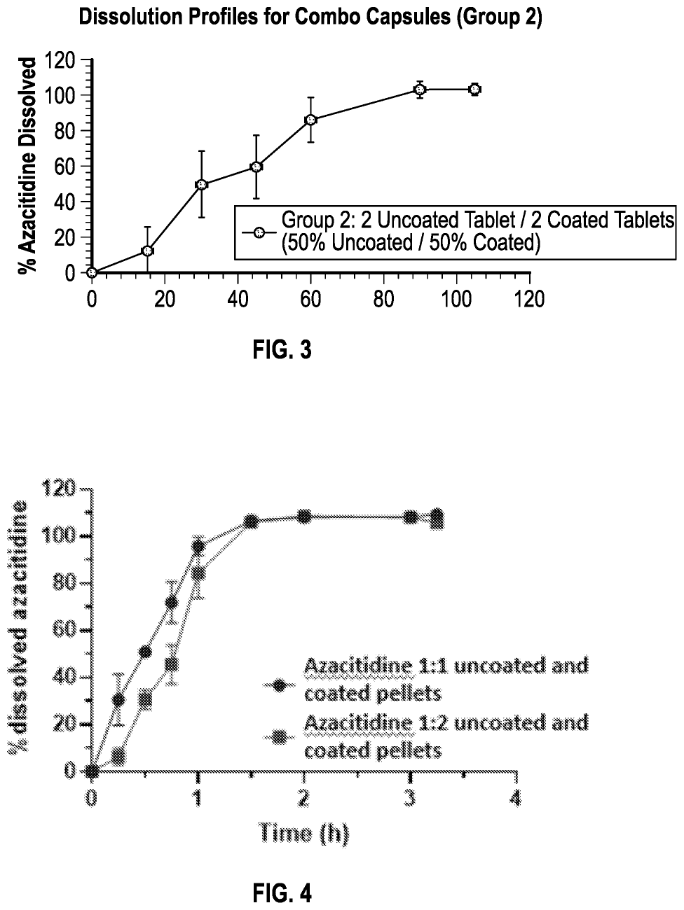

For the combination of coated and uncoated azacitidine tablets in a capsule, dissolution profile for the combination of 2 uncoated azacitidine minitablets, and rest 2 coated azacitidine minitablets was tested under same condition. The drug release data was collected at 15, 30, 45, 60, 90, and 105 minutes. The release date for azacitidine is shown in FIG. 3.

FIG. 4 shows the dissolution release profile of azacitidine from a dosage form comprising coated and uncoated azacitidine minitablets in ratios of 1:1 and 1:2 (uncoated:coated), at pH 6.8. As shown in FIG. 4, the dosage form with more coated azacitidine minitablets exhibited more delayed release of azacitidine.

### Example 3: Uncoated Cedazuridine 20 mg Tablet & 4 mg Azacitidine (Intragranular and Extragranular Layers) Minitablets

### Manufacturing Process

20 mg cedazuridine round tablet was prepared by direct compression. The composition of the cedazuridine core is presented in Table 3-1. Individual excipients and cedazuridine, except magnesium stearate, were screened through 30 mesh and blended for a 15 minutes at 25 rpm in a 1.5 L V-blender. The blended cedazuridine and excipients were then mixed with hand screened magnesium stearate and blended again for 3 minutes at 25 rpm. The final blend was compressed into round (0.25″ inch) tablet by direct compression on a Korsch XL-100 press. Cedazuridine tablet was not coated and used as immediate release minitablets.

TABLE 3-1
20 mg Cedazuridine tablet composition

Cedazuridine 20 mg

| Ingredients | mg/tab | % |
|---|---|---|
| Cedazuridine | 20.0 | 20.0 |
| Lactose monohydrate | 71.5 | 71.5 |
| (FlowLac ® 90) | | |
| Croscarmellose Sodium (Ac- | 5.0 | 5.0 |
| Di-Sol ® SD-711) | | |
| Hypromellose E5 2910 | 2.0 | 2.0 |
| Colloidal Silicon Dioxide | 1.0 | 1.0 |
| Magnesium Stearate | 0.5 | 0.5 |
| (Hyqual ®, Vegetal) | | |
| Core tablet total, Oval | 100 mg | 100 |

Azacitidine minitablets having intragranular and extragranular layers, having two different composition of Table 3-2 (“Core 1” and “Core 2”), were prepared. Azacitidine and all intragranular excipients were dispensed into individual containers and screened through 30 mesh. Sieved azacitidine and intragranular excipients except for magnesium stearate were added into a blender with a suitable capacity and mixed at 25 rpm at predetermined duration. Magnesium stearate for intragranular layer was then sieved and added to the blend, and the lubrication blending was performed. The intragranular blend was then roller compacted into ribbons and ribbons were milled into granules. Milled granules were blended with screened extra granular excipients to make final blend. The final blend was compressed into minitablets tablets using 2.5-mm round, plain face tooling. The target weight of the tablets was 10 mg±10% and a target hardness range was between 1-3 kp.

TABLE 3-2
4 mg Azacitidine minitablet core composition

| | Core 1 | | Core 2 | |
|---|---|---|---|---|
| Ingredients | mg/tab | % | mg/tab | % |

Intragranular

| | | | | |
|---|---|---|---|---|
| Azacitidine | 4.0 | 40.0 | 4.03 | 40.0 |
| MCC (PH 102) | 2.5 | 25.0 | 2.50 | 25.0 |
| Lactose Monohydrate (Flow | 1.99 | 19.9 | 1.94 | 19.4 |
| Lac 90) | | | | |
| Croscarmellose Sodium | 0.25 | 2.5 | 0.25 | 2.5 |
| Hypromellose E6 | — | — | 0.20 | 2.0 |
| Kollidone VA64 | 0.2 | 2.0 | — | — |
| Aerosil 200 or CabOsil | 0.05 | 0.5 | 0.05 | 0.5 |
| (Colloidal silicon dioxide) | | | | |
| Magnesium Stearate | 0.08 | 0.8 | 0.08 | 0.8 |

Extragranular

| | | | | |
|---|---|---|---|---|
| Avicel PH 200 (Microcrystalline | — | — | 0.50 | 5.0 |
| cellulose) | | | | |
| Avicel PH 102 (Microcrystalline | 0.4 | 4.0 | | |
| cellulose) | | | | |
| Croscarmellose sodium | 0.25 | 2.5 | 0.25 | 2.5 |
| Colloidal silicon dioxide | 0.05 | 0.5 | 0.16 | 1.6 |
| Magnesium stearate | 0.02 | 0.2 | 0.04 | 0.4 |
| Kollidon VA64 | 0.21 | 2.1 | — | — |
| Core minitablet total, | 10.0 mg | 100 | 10.0 mg | 100 |
| minitablet (2 mm) | | | | |

The uncoated tablets were seal coated at 5% weight gain followed by functional coating of Surelease to modulate the dissolution profile. HPMC from Coloron (Opadry® complete coating system YS-1-19025-A-Clear) was used both for seal coat and as a pore former in an ethyl cellulose dispersion (Surelease) for a functional coat. “Core 1” minitablets were coated with 75:25 (Surelease®: Pore Former) at 20% wt. gain and “Core 2” minitablets were coated with 80:20 Surelease: Pore Former coating at 15% wt. gain. Composition of coating is listed in Tables 3-3 and 3-4. Both seal coating and functional coating of minitablets were performed in a Wuster coater.

TABLE 3-3
Seal Coat composition (200 mL preparation, 10% solids)

| | Theoretical |
|---|---|
| Material | amount (g) |
| Opadry ® complete film coating system YS-1-1-19025- | 20 |
| A-Clear | |
| Water | 180 |

TABLE 3-4
Surelease composition (500 mL preparation, 10% solids)

| | Theoretical amount | Theoretical amount |
|---|---|---|
| | (g) for 75/25 | (g) for 80/20 |
| | (Surelease:Pore | (Surelease:Pore |
| Material | Former) | Former) |
| Opadry ® complete film | 12.5 | 10.0 |
| coating system YS-1-1-19025- | | |
| A-Clear | | |
| Surelease ®* | 150.0 | 160.0 |
| Water | 337.5 | 330.0 |

*Surelease dispersion at 25% solids

### Dissolution Profile

For dissolution testing, five (5) coated or uncoated azacitidine minitablets at each weight gain (15% and 20%) were placed in a size 0, VCAPS Plus capsule for a total dose of 20 mg and tested for dissolution. Dissolution testing was carried out in 500 mL, pH 6.8, 50 mM sodium phosphate buffer at 37° C. bath temperature with an agitation speed of 75 rpm in a basket. The drug release data was collected at 0, 30, 45, 60, 90, 120, 180 mins and at 195 mins (deemed infinity) with 250 rpm speed. The release profile for all 3 groups is presented in FIG. 6.

As shown in FIG. 6, the dissolution results showed uncoated azacitidine minitablets released azacitidine within 30 mins followed by 15% coated azacitidine minitablets at 80/20 Surelease: Pore former ratio and then 20% coated azacitidine minitablets with 75/25 Surelease: pore former ratio. The drug release extended to 90 mins and 120 mins with 15% and 20% coated minitablets, respectively.

### Example 4: Uncoated Cedazuridine 20 mg Tablet & 3 Layer Coated 4 mg Azacitidine (Intragranular and Extragranular Layers) Minitablets

### Manufacturing Method

The uncoated cedazuridine 20 mg minitablets and the uncoated azacitidine 4 mg minitablets were prepared using the method described in Example 3. The composition for cedazuridine and azacitidine minitablets are shown in Tables 4-1 and 4-2.

TABLE 4-1
20 mg Cedazuridine tablet composition

Cedazuridine 20 mg

| Ingredients | mg/tab | % |
|---|---|---|
| Cedazuridine | 20.0 | 20.0 |
| Lactose monohydrate | 71.5 | 71.5 |
| (FlowLac ® 90) | | |
| Croscarmellose Sodium (Ac- | 5.0 | 5.0 |
| Di-Sol ® SD-711) | | |
| Hypromellose E5 2910 | 2.0 | 2.0 |
| Colloidal Silicon Dioxide | 1.0 | 1.0 |
| Magnesium Stearate | 0.5 | 0.5 |
| (Hyqual ®, Vegetal) | | |
| Core minitablet total, Oval | 100 mg | 100 |

TABLE 4-2
Formulation Composition of 4 mg Azacitidine minitablets

| | | | Unit dose |
|---|---|---|---|
| Material | Function | % w/w | mg/tablet |
| Azacitidine | API | 40.0 | 4.0 |
| Lactose monohydrate, Flowlac | Diluent | 19.9 | 1.99 |
| 90 | | | |
| Microcrystalline cellulose, | Diluent | 25.0 | 2.5 |
| PH 102 | | | |
| Croscarmellose sodium, | Disintegrant | 2.5 | 0.25 |
| AcDiSol SD-711 | | | |
| Kollidon VA 64 | Binder | 2.0 | 0.2 |
| Colloidal silicon dioxide | Glidant | 0.5 | 0.05 |
| Magnesium Stearate, Emprove, | Lubricant | 0.8 | 0.08 |
| Vegetable | | | |

| | | |
|---|---|---|
| Total Intragranular | 90.7 | 9.07 |

Extragranular

| | | | |
|---|---|---|---|
| Microcrystalline cellulose, | Diluent | 4.0 | 0.4 |
| PH 200 | | | |
| Croscarmellose sodium, | Disintegrant | 2.5 | 0.25 |
| AcDiSol SD-711 | | | |
| Kollidon VA 64 | Binder | 2.1 | 0.21 |
| Colloidal silicon dioxide, | Glidant | 0.5 | 0.05 |
| Aerosil 200 | | | |
| Magnesium Stearate, Emprove, | Lubricant | 0.2 | 0.02 |
| Vegetable | | | |

| | | |
|---|---|---|
| Total Extragranular | 9.3 | 0.93 |
| Core minitablet total, round | 100 | 10 |

The uncoated azacitidine minitablets were seal coated at 2% weight gain followed by intermediate coating at 20% wt. gain, and a final coat of Eudragit® polymer at two polymer weight gains of 5.3% which corresponds to 8.5% total weight gain.

Both seal coating and functional coating of minitablets were performed in a Wuster coater. The composition of seal, intermediate and functional coatings is presented in Tables 4-3, 4-4 and 4-5 respectively.

TABLE 4-3
Composition for seal coating for 2% wt. gain

HPMC E3

| Ingredients | Quantity (g) | % |
|---|---|---|
| HPMC E3 (JRS Pharma) | 6.0 | 6.82 |
| PEG 600 (TCI) | 0.6 | 0.68 |
| Purified water | 81.4 | 92.5 |
| Total | 88.0 | 100.0 |

| | |
|---|---|
| Solid content | 7.5 |

TABLE 4-4
Composition for intermediate coating for 20% wt. gain

HPMC E3

| Ingredients | Quantity (g) | % |
|---|---|---|
| HPMC E3 (JRS Pharma) | 30.0 | 7.1 |
| Magnesium Oxide Light | 30.0 | 7.1 |
| PEG 600 (TCI) | 3.0 | 0.7 |
| Purified water | 357.0 | 85.0 |
| Total | 420.0 | 100.0 |

| | |
|---|---|
| Solid content | 15.0 |

TABLE 4-5
Composition of enteric Functional Coating

| Ingredients | Quantity (g) | % |
|---|---|---|
| Eudragit ® L30D-55 | 75.0 (22.5 g of | 12.5 (solid |
| (Evonik) | solid) | basis) |
| Triethyl Citrate | 2.25 | 1.25 |
| Talc | 11.25 | 6.25 |
| Purified water | 91.5 | 50.8 |
| Total | 180.0 | 100.0 |

| | |
|---|---|
| Solid content | 20.0 |

### Dissolution Testing

For dissolution testing, five (5) coated azacitidine minitablets at 5.3% polymer weight gain were placed in a size 1, VCAPS plus capsule for a total dose of 20 mg and tested for dissolution.

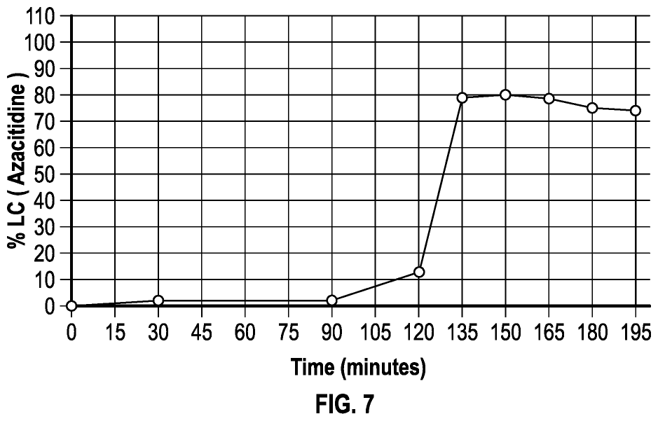

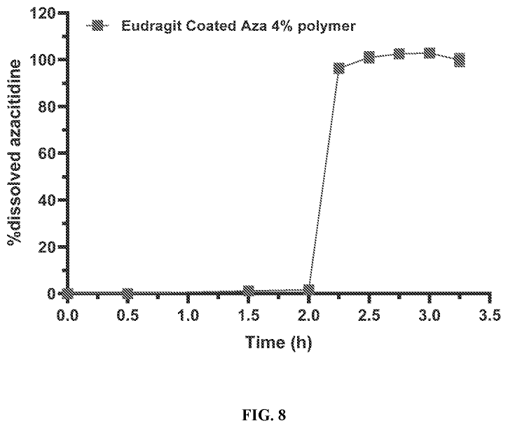

Dissolution testing was carried out in two stages, acid stage, pH 1.2, 0.1N HCl and buffer stage, pH 6.8, 50 mM phosphate buffer at 37° C. bath temperature with an agitation speed of 75 rpm in a basket and 500 mL dissolution media. The minitablets coated with Eudragit® polymer prevented the release of azacitidine in the acid stage followed by rapid release in the buffer stage, 6.8 pH after media switch at 120 mins (FIG. 7). As shown in FIG. 7, azacitidine was not released for the first 2 hours in acidic condition, while azacitidine was mostly released after being moved to pH 6.8, confirming the pH sensitivity of the Eudragit® L30D coating. Further, Eudragit® coated minitablets at 5.3% wt. gain provided enough protection in the acid stage followed by rapid release in the buffer stage at pH 6.8 after media switch at 120 minutes.

### Example 5-1: Uncoated Cedazuridine 20 mg Tablet & 2 Layer Coated 4 mg Azacitidine (Intragranular and Extragranular Layers) Minitablets

The uncoated cedazuridine 20 mg tablet of Example 4 were provided. Azacitidine and all intragranular excipients except magnesium stearate listed in Table 5-1 were dispensed into 8 Qt. V Shell equipped on PK blender and mixed for 10 minutes at 25 rpm. The preblend was screened through a Quadro Comil Model U3 equipped with a 032R screen and a round impeller at a speed 4500+/−100 rpm. The screened material was transferred back into the blender and mixed again for 10 minutes at 25 rpm. Magnesium stearate was hand screened through 30 mesh and added into a blender and mixed for 10 mins at 25 rpm. The intragranular blend was roller compacted on a Alexanderwerk roller compactor at roller speed of 4.0 rpm, compaction force of 4.0 KN/cm and feed screw speed at 20 rpm. The ribbons were milled on a granulator at speed 100 rpm speed equipped with 0.80 mm screen. The extragranular excipient quantity was adjusted based on the yield of intragranular milled granules. Extragranular excipients were hand screened through 30 mesh. Both intragranular milled granules and extragranular excipients except magnesium stearate were transferred into the blender and mixed for 10 minutes at 25 rpm. Screened magnesium stearate was added to the blended material and mixed for 3 minutes at 25 rpm. The final blend was compressed into minitablets using round tooling of 2.5 mm diameter on a Natoli RD30 press. The target weight of the minitablets was 10 mg±1.0 mg and hardness 1-3 KP as determined on a Hardness Testor by Scotax.

TABLE 5-1
Azacitidine 4 mg uncoated minitablets

| | | |
|---|---|---|
| Ingredient | mg | % |

Intragranular

| | | |
|---|---|---|
| Azacitidine | 4.0 | 40.0 |
| Lactose monohydrate | 1.97 | 19.7 |
| MCC (Avicel ® PH-102) | 2.50 | 25.0 |
| Croscarmellose Sodium | 0.25 | 2.5 |
| Hypromellose | 0.20 | 2.0 |
| Colloidal silicon Dioxide | 0.05 | 0.5 |
| Magnesium Stearate | 0.08 | 0.8 |
| Intragranular Total | 9.05 | 90.5 |

Extragranular

| | | |
|---|---|---|
| MCC (Avicel ® PH-102) | 0.5 | 5.0 |
| Croscarmellose Sodium | 0.25 | 2.5 |
| Colloidal Silicon Dioxide | 0.16 | 1.6 |
| Magnesium Stearate | 0.04 | 0.4 |
| Extragranular Total | 0.95 | 9.5 |
| Core minitablet total | 10.0 | 100.0 |

The uncoated azacitidine 4 mg minitablets were coated using ingredients listed in Tables 5-2 and 5-3, using the method described in Example 2. Weight gains of 15% or 20% were employed.

TABLE 5-2
Seal coating composition

| Ingredient | % |
|---|---|
| Opadry Clear, YS-1-19025-A (HPMC based) | 10 |
| Purified water | 90 |

TABLE 5-3
Release modulating coat

| Ingredient | % |
|---|---|
| Opadry clear, YS-1-19025-A (HPMC based) | 2.0-2.5 |
| Surelease ® E-7-19040 (Ethyl cellulose based) | 7.5-8.0 |
| Milli-Q water | 90 |

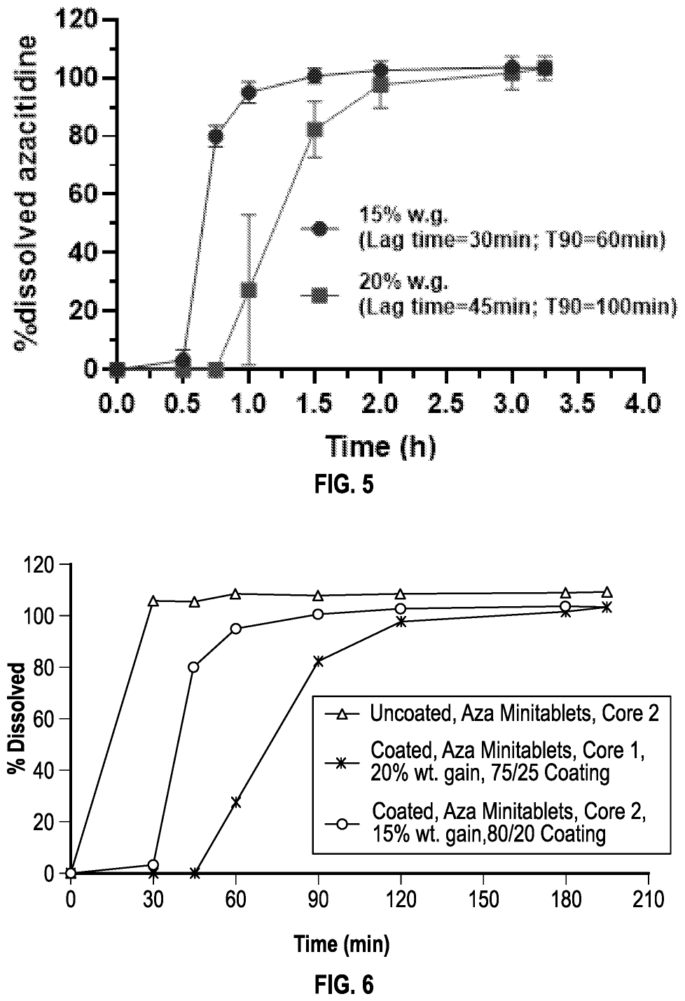

Dissolution profile of the coated azacitidine minitablets (15% and 20% weight gain) was measured using the method described in Example 2. The drug release data was collected at 0, 30, 45, 60, 90, 120, 180 mins and at 195 mins (deemed infinity). FIG. 5 shows the dissolution release profile of azacitidine at pH 6.8.

### Example 5-2: Uncoated Cedazuridine 20 mg Tablet & 3 Layer Coated 4 mg Azacitidine (Intragranular and Extragranular Layers) Minitablets

Instead of coatings as described in Example 5-1, the uncoated azacitidine 4 mg minitablets comprising intragranular and extragranular layers were seal coated in two layers, using ingredients listed in Tables 5-4 (first layer) and 5-5 (second layer). The seal coated azacitidine pellets were further coated with delayed release coating, using ingredients listed in Table 5-6.

TABLE 5-4
Seal coat first layer

| Ingredient | % |
|---|---|
| HPMC E5 | 7.5 |
| Purified water | 92.5 |

TABLE 5-5
Seal coat second layer

| Ingredient | % |
|---|---|
| HPMC E3 | 7.5 |
| Magnesium oxide, light | 7.5 |
| Purified water | 85 |

TABLE 5-6
Delayed release coating

| Ingredient | % |
|---|---|
| Eudragit ® L30D-55 | 41.7 |
| Triethyl citrate | 1.25 |
| Talc | 6.25 |
| Purified water | 50.8 |

### Example 6: Uncoated Cedazuridine 20 mg Tablet & 2 Layer Coated 4 mg Azacitidine (Intragranular and Extragranular Layers) Minitablets

### Manufacturing Process

20 mg cedazuridine round tablets were prepared by direct compression. The composition of cedazuridine core tablets is listed in Table 6-1. Individual excipients and cedazuridine except magnesium stearate were screened through 30 mesh and blended in a V-blender, 1.5 L for 15-20 minutes. Blended cedazuridine and excipients were then mixed with screened magnesium stearate and blended again in a V-blender for 5 minutes. Cedazuridine tablets were compressed into round (0.25″ inch) tablets by direct compression on a Korsch XL-100 press.

TABLE 6-1
20 mg Cedazuridine tablet composition

Cedazuridine 20 mg

| Ingredients | mg/tab | % |
|---|---|---|
| Cedazuridine | 20.0 | 20.0 |
| Lactose monohydrate | 71.5 | 71.5 |
| (FlowLac ® 90) | | |
| Croscarmellose Sodium (Ac- | 5.0 | 5.0 |
| Di-Sol ® SD-711) | | |
| Hypromellose E5 2910 | 2.0 | 2.0 |
| Colloidal Silicon Dioxide | 1.0 | 1.0 |
| Magnesium Stearate | 0.5 | 0.5 |
| (Hyqual ®, Vegetal) | | |
| Core tablet total, Oval | 100 mg | 100 |

Azacitidine and all intragranular excipients were dispensed into individual containers and screened through 30 mesh. The composition of azacitidine core tablets is listed in Table 6-2. Sieved azacitidine and intragranular excipients except for magnesium stearate were added into a 5 L Bohle blender and mixed at 25 rpm for 10 mins. Magnesium stearate was then sieved and added to the blend, and the lubrication blending was performed for 3 mins at 25 rpm in a 5 L Bohle blender. The intragranular blend was roller compacted into ribbons on a Gereteis FMX1064 and ribbons were milled into granules. Milled granules were blended with screened extra granular excipients except magnesium stearate in the same 5 L Bohle blender for 10 minutes at 25 rpm. Screened magnesium stearate was added to the rest of the blend and mixed again for 3 minutes at 25 rpm. The final blend was compressed into minitablets using 2.5-mm round, plain face tooling. The target weight of the tablets was 10 mg±10% and a target hardness range between 1-3 kp.

TABLE 6-2
Azacitidine 4 mg uncoated minitablets

| | | |
|---|---|---|
| Ingredient | mg | % |

Intragranular

| | | |
|---|---|---|
| Azacitidine | 4.0 | 40.0 |
| Lactose monohydrate (FlowLac 90) | 1.97 | 19.7 |
| MCC (Avicel ® PH-102) | 2.50 | 25.0 |
| Croscarmellose Sodium (Ac-di-sol SD-711) | 0.25 | 2.5 |
| Hypromellose E5 | 0.20 | 2.0 |
| Colloidal silicon Dioxide | 0.05 | 0.5 |
| Magnesium Stearate (Emprove, Vegetable) | 0.08 | 0.8 |
| Intragranular Total | 9.05 | 90.5 |

Extragranular

| | | |
|---|---|---|
| MCC (Avicel ® PH-200) | 0.5 | 5.0 |
| Croscarmellose Sodium (AcDiSol SD-711) | 0.25 | 2.5 |
| Colloidal Silicon Dioxide (Aerosil 200) | 0.16 | 1.6 |
| Magnesium Stearate, Emprove, Vegetable | 0.04 | 0.4 |
| Extragranular Total | 0.95 | 9.5 |
| Core minitablet total | 10.0 | 100.0 |

The uncoated tablets were seal coated at 2% weight gain followed functional coating with Eudragit® polymer (L30D-55) at polymer weight gain 4.0%. Both seal and functional coating of minitablets were performed in a Wurster coater, GPCG2, 3 L. The composition of seal coat and enteric coat is presented in Table 38, and 40, and process parameters are in Tables 6-3 and 6-4.

TABLE 6-3
Azacitidine Seal Coat Composition

| Ingredient | Supplier | Quantity (g) |
|---|---|---|
| HPMC E3 | Viva Pharma | 12 |
| PEG 600 | TCI | 12 |
| Purified water | | 162.8 |
| Total dispersion | | 176.8 |

TABLE 6-4
Azacitidine Functional Coat Composition

| Ingredient | Supplier | Quantity (g) |
|---|---|---|
| Eudragit ® L30 D-55 | Evonik | 150 |
| Triethyl citrate | Spectrum | 4.5 |
| Talc | Spectrum | 22.5 |
| Purified water | | 183 |
| Total dispersion | | 360 |

### Dissolution Testing

For dissolution testing, five (5) coated azacitidine minitablets at 4.0% polymer weight gain were placed in a size 1, VCAP plus capsule for a total dose of 20 mg and tested for dissolution. Dissolution testing was carried out in two stages, acid stage, pH 1.2, 0.1N HCl and buffer stage, pH 6.8, 50 mM phosphate buffer at 37° C. bath temperature with an agitation speed of 75 rpm in a basket and 500 mL dissolution media. As shown in FIG. 8, the minitablets coated with Eudragit® polymer prevented the release of azacitidine in the acid stage followed by rapid release in the buffer stage, 6.8 pH after media switch at 120 mins.

### Example 7: Uncoated Cedazuridine 100 mg Tablet and Coated Azacitidine 4 mg Minitablets

Uncoated cedazuridine 100 mg tablets were prepared using the ingredients listed in Table 7-1, using the method described in Example 6. Eudragit® coated azacitidine 4 mg minitablets were prepared using the ingredients listed in Table 7-2.

TABLE 7-1
Cedazuridine 100 mg Uncoated Tablet

| Ingredient | mg | % |
|---|---|---|
| Cedazuridine | 100.00 | 20.00 |
| Lactose monohydrate | 357.50 | 71.50 |
| Croscarmellose Sodium | 25.00 | 5.00 |
| Hypromellose | 10.00 | 2.00 |
| Colloidal silicon Dioxide | 5.00 | 1.00 |
| Magnesium Stearate | 2.50 | 0.50 |
| Total | 500.00 | 100.00 |

TABLE 7-2
4 mg Azacitidine minitablet with coating composition

| | | |
|---|---|---|
| Ingredient | mg/tab | (%) |

Tablet core: Intragranular

| | | |
|---|---|---|
| Azacitidine | 4.0 | 36.24 |
| Microcrytalline cellulose (PH-102) | 2.50 | 22.65 |
| Lactose Monohydrate | 1.97 | 17.85 |
| Croscarmellose Sodium | 0.25 | 2.27 |
| Hypermellose 2910 | 0.20 | 1.81 |
| Colloidal silicon dioxide | 0.05 | 0.45 |
| Magnesium Stearate | 0.08 | 0.73 |
| Intragranular total | 9.05 | 82.00 |

Tablet core: Extragranular

| | | |
|---|---|---|
| Microcrystalline cellulose (PH-200) | 0.50 | 4.53 |
| Croscarmellose sodium | 0.25 | 2.26 |
| Colloidal silicon dioxide | 0.16 | 1.45 |
| Magnesium stearate | 0.04 | 0.36 |
| Extragranular total | 0.95 | 8.60 |
| Tablet core total | 10.00 | 90.60 |

Seal coat (applied on minitablet)

| | | |
|---|---|---|
| Hypromellose | 0.20 | 1.81 |
| Polyethylene glycol 600 | 0.02 | 0.18 |
| Seal coat total | 0.22 | 1.99 |

Enteric coat (applied on minitablet)

| | | |
|---|---|---|
| Eudragit ® L30D 55 | 0.51 | 4.63 |
| Triethyl citrate | 0.05 | 0.46 |
| Talc | 0.26 | 2.32 |
| Enteric coat total | 0.82 | 7.41 |
| Total coated minitablet | 11.04 | 100.00 |

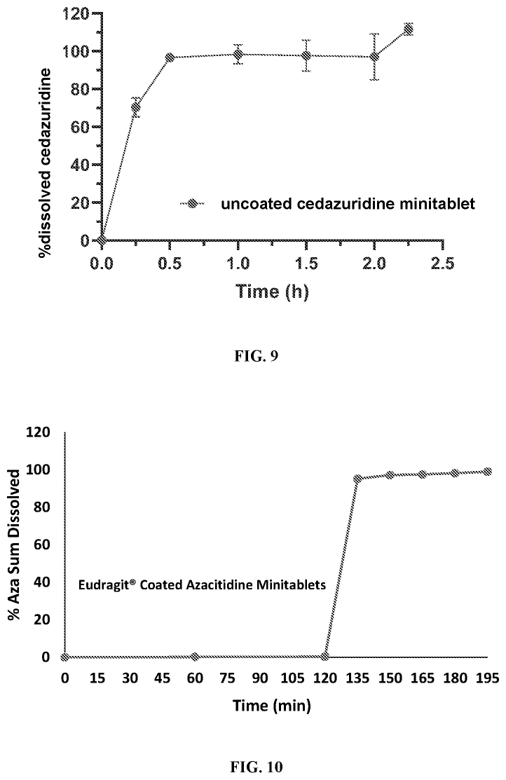

For dissolution testing, the 100 mg uncoated cedazuridine tablet was placed in a size 0, Vcaps® Plus capsule and tested for dissolution. Dissolution testing was carried out in two stages, acid stage, pH 1.2, 0.1N HCl and buffer stage, pH 6.8, 50 mM phosphate buffer at 37° C. bath temperature with an agitation speed of 75 rpm in a basket and 900 mL dissolution media, where the switch was occurred at 120 min. The drug release data was collected at 0, 15, 30, 60, 90, 120, and at 135 mins (deemed infinity). The release profile is presented in FIG. 9. As shown in FIG. 9, the uncoated cedazuridine tablet are all dissolved in acid stage in about 30 minutes, before media switch.

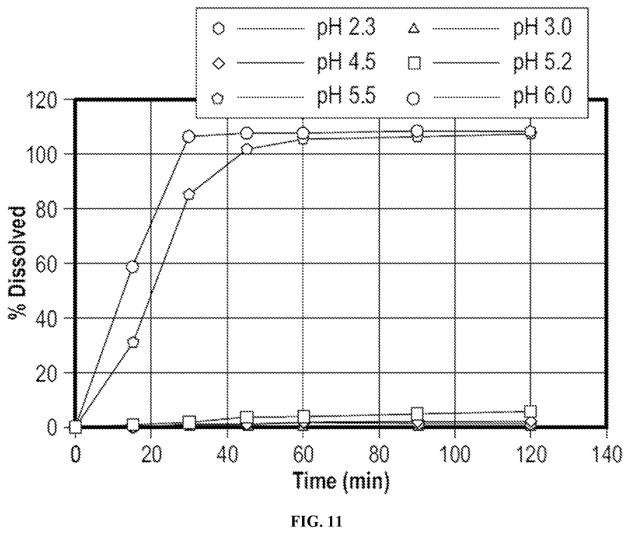

For dissolution testing, five (5) enteric coated azacitidine minitablets were placed in a size 0, Vcaps® Plus capsule and tested for dissolution. Dissolution testing was carried out in two stages, acid stage, pH 1.2, 0.1N HCl and buffer stage, pH 6.8, 50 mM phosphate buffer at 37° C. bath temperature with an agitation speed of 75 rpm in a basket and 500 mL dissolution media. The minitablets coated with Eudragit® polymer prevented the release of azacitidine in the acid stage followed by rapid release in the buffer stage, 6.8 pH after media switch at 120 mins. The drug release data was collected at 0, 60, 120, 135, 150, 165, 180 mins and at 195 mins (deemed infinity). The release profile is presented in FIG. 10. As shown in FIG. 10, the minitablets coated with Eudragit® polymer prevented the release of azacitidine in the acid stage followed by rapid release in the buffer stage, 6.8 pH after media switch at 120 mins.

To test dissolution of the enteric coated azacitidine minitablets at various pHs, five (5) enteric coated azacitidine minitablets were placed in a size 0, Vcaps® Plus capsule and tested for dissolution. Dissolution testing was carried out in 6 groups in each of pH 2.3, 3.0, 4.5, 5.2, 5.5, and 6.0 buffers, at 37° C. bath temperature with an agitation speed of 75 rpm in a basket. The drug release data was collected at 0, 15, 30, 45, 60, 90 and 120 mins (deemed infinity) with 250 rpm speed. The release profile for all 6 groups is presented in FIG. 11. As shown in FIG. 11, Example 8: Preparation of a Blended Powder of Cedazuridine

A cedazuridine blended powder having the composition of Table 8-1 was prepared and can be optionally used in capsules instead of uncoated tablets of cedazuridine. Cedazuridine and all the other excipients except magnesium stearate were hand screened through 30 mesh and transferred to a 5 L Bohle Blender and mixed for 10 minutes at 25 rpm. Magnesium stearate was hand screened through 30 mesh and transferred to the blender and mixed with the drug and other excipients for 3 minutes at 25 rpm. The cedazuridine powder blend formulation can be filled in a capsule.

TABLE 8-1

| Material Name | % w/w Solids | mg/Cap |
|---|---|---|
| Cedazuridine | 80.0 | 100.000 |
| Lactose monohydrate (FloLac ® 90) | 13.5 | 16.875 |
| Croscarmellose sodium | 5.0 | 6.250 |
| Silicon dioxide (Aerosil ® 200) | 1.0 | 1.250 |
| Magnesium stearate | 0.5 | 0.625 |
| ConiCap HG Capsule Shell Size 1 (Optional) | | 1 EA |
| Total | 100 | 125 |

### Example 9: Preparation of Capsules Comprising Combination of Example 7

The uncoated 100 mg cedazuridine tablet (from Example 7) and ten 4 mg azacitidine coated minitablets (from Example 7) were placed in a Vcaps® Plus capsule. The resulting capsule had the composition listed in Table 9.

TABLE 9
Azacitidine 40 mg/Cedazuridine 100 mg capsule composition

Azacitidine 40 mg/
Cedazuridine 100 mg Capsule

| Ingredients | mg/capsule | % w/w |
|---|---|---|
| Cedazuridine | 100.0 | 16.4 |
| Azacitidine | 40.0 | 6.6 |
| Lactose monohydrate | 377.2 | 61.8 |
| MCC (Avicel PH102) | 25.0 | 4.1 |
| MCC (Avicel PH200) | 5.0 | 0.8 |
| Croscarmellose Sodium | 30.0 | 4.9 |
| Hypromellose | 14.0 | 2.3 |
| Colloidal Silicon Dioxide | 7.1 | 1.2 |
| Magnesium Stearate | 3.7 | 0.6 |
| Polyethylene glycol 600 | 0.2 | 0.03 |
| Eudragit ® L30D 55 | 5.1 | 0.84 |
| Triethyl citrate | 0.5 | 0.08 |
| Talc | 2.6 | 0.43 |
| Total | 610.4 | 100 |

### Example 10: Preparation of Capsules Comprising Cedazuridine Powder and Coated Azacitidine Minitablets

The 100 mg cedazuridine powder of Example 8, and ten of the coated 4 mg azacitidine minitablets of Example 7 are placed in a Vcaps® Plus capsule. For dissolution testing, the 100 mg cedazuridine powder is placed in a size 0, Vcaps® Plus capsule and tested for dissolution. Dissolution testing is carried out in two stages, acid stage, pH 1.2, 0.1N HCl and buffer stage, pH 6.8, 50 mM phosphate buffer at 37° C. bath temperature with an agitation speed of 75 rpm in a basket and 900 mL dissolution media, where the switch is occurred at 120 min. The drug release data is collected at 0, 30, 45, 60, 90, 120, 180 mins and at 195 mins (deemed infinity).

For dissolution testing, the enteric coated azacitidine minitablets are placed in a size 0, Vcaps® Plus capsule and tested for dissolution. Dissolution testing is carried out in two stages, acid stage, pH 1.2, 0.1N HCl and buffer stage, pH 6.8, 50 mM phosphate buffer at 37° C. bath temperature with an agitation speed of 75 rpm in a basket and 500 mL dissolution media. The drug release data is collected at 0, 30, 45, 60, 90, 120, 180 mins and at 195 mins (deemed infinity).

### Example 11: Enteric Capsule Filled with Azacitidine and Cedazuridine

Approximately 8 to 10 gram batches of cedazuridine and azacitidine powder mixtures were prepared and filled in capsule shells. The compositions of the powders are provided in the table below.

Cedazuridine and azacitidine, in powder form, half of lactose monohydrate, half of Avicel® PH-102, and the remaining excipients except for magnesium stearate were individually screened through a 0.6-mm sieve and transferred into an 8-oz container.

The remaining lactose monohydrate and Avicel® PH-102 were used to dry wash container of cedazuridine and decitabine, respectively. Half of the total quantity of Avicel® PH-102 and lactose monohydrate listed in the table below was used for the dry wash. After the dry wash, both excipients were screened through a 0.6-mm sieve and added into the blend container. The pre-blending of the mixture was performed at 25 rpm for 15 minutes in a Turbula Shaker, followed by sieving through a 0.6-mm screen, and further blending at 25 rpm for 15 minutes. The weight of resulting blend was weighed and used to adjust the amount of magnesium stearate, which was hand-sieved through a 0.6-mm screen. The sieved magnesium stearate was then added to the blend, and blending was carried out at 25 rpm for 3 minutes.

The final blend was manually filled into Vcaps® enteric capsule shell (purchased from Capsugel) at the pre-determined fill weight of 200 mg±3 mg per capsule.

The capsules were packaged into 30-cc white HDPE bottles with 8 counts per bottle with 2×1-g desiccant canister. The bottles were capped with a 28-mm child-resistance closure and induction sealed.

Ingredients and weight proportions for capsules comprising 20 mg of azacitidine and 20 mg of cedazuridine are provided in Table 11 below.

TABLE 11
20 mg azacitidine/20 mg cedazuridine FDC capsule

| | | | | mg per 8 g |
|---|---|---|---|---|
| Ingredient | Supplier | mg/capsule | % w/w | blend |
| Azacitidine | n/a | 20.0 | 10.0 | 800.0 |
| Cedazuridine | n/a | 20.0 | 10.0 | 800.0 |
| Sodium phosphate, | Sigma | 30.0 | 15.0 | 2092.3 |
| anhydrous | Aldrich | | | |
| Lactose monohydrate | Meggle | 52.4 | 26.2 | 1302.0 |
| (FlowLac ® 90) | Pharma | | | |
| MCC (Avicel ® PH-102) | Dupont | 32.6 | 16.3 | 749.8 |
| MCC (Avicel ® PH-200) | Dupont | 18.8 | 9.4 | 1200.0 |
| Croscarmellose Sodium | Dupont | 16.0 | 8.0 | 640.0 |
| (Ac-Di-Sol ®) | | | | |
| Hypromellose E6 | JRS | 4.0 | 2.0 | 160.0 |
| (VIVAPHARM ®) | Pharma | | | |
| Colloidal | Evonik | 4.2 | 2.1 | 168.0 |
| silicon Dioxide | | | | |
| (Aerosil ® 200) | | | | |

### Example 12: FDC Cedazuridine and Azacitidine Tablets

Both azacitidine and cedazuridine were formulated for immediate release. 20 mg azacitidine and cedazuridine fixed dose combination (FDC) oval tablets with core tablet weight 500 mg (Table 12), using the method described in Example 1.

TABLE 12
Composition of oval FDC tablets containing
20 mg cedazuridine and 20 mg azacitidine
Core Tablet Composition

Azacitidine 20 mg/
Cedazuridine 20 mg

| | | |
|---|---|---|
| Ingredients | mg/tab | % |

Intragranular

| | | |
|---|---|---|
| Azacitidine | 20.0 | 4.0 |
| Cedazuridine | 20.0 | 4.0 |
| Lactose monohydrate (FlowLac ® 90) | 206.6 | 41.3 |
| MCC (Avicel ® PH-102) | 128.4 | 25.7 |
| Croscarmellose Sodium | 12.5 | 2.5 |
| (Ac-di-sol ® SD-711) | | |
| Hypromellose E5 2910 | 10.0 | 2.0 |
| Colloidal Silicon Dioxide | 2.7 | 0.54 |
| Magnesium Stearate (Hyqual ®, Vegetal) | 4.0 | 0.8 |
| Intragranular Total | 404.2 | 80.8 |

Extragranular

| | | |
|---|---|---|
| MCC (Avicel ® PH-200) | 74.0 | 14.8 |
| Croscarmellose Sodium | 12.5 | 2.5 |
| (Ac-di-sol ® SD-711) | | |
| Colloidal Silicon Dioxide | 8.0 | 1.6 |
| Magnesium Stearate. (Hyqual ®, Vegetal) | 1.3 | 0.26 |
| Extragranular Total | 95.8 | 19.2 |
| Core tablet total, Oval | 500.0 | 100.0 |

### Example 13: Pharmacokinetics (PK) Study in Monkeys Using Dosage Forms of Example 1

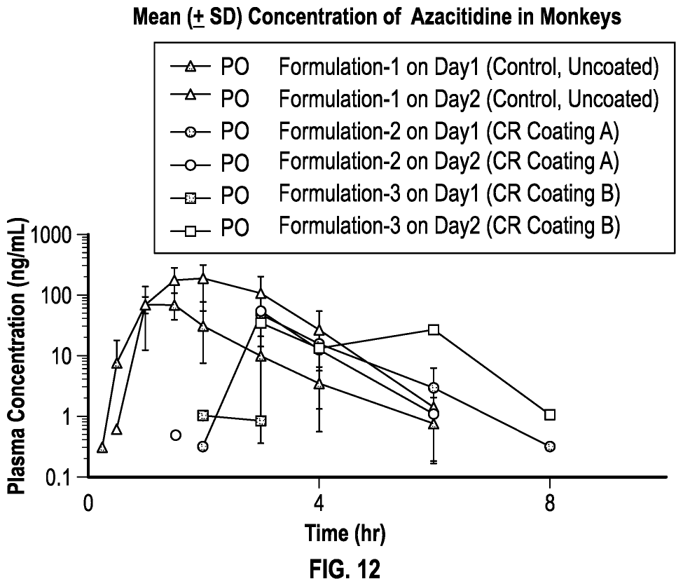

Twelve monkeys were grouped into three groups, 4 monkeys each, and dosed with the following: Group 1-uncoated 20 mg cedazuridine/20 mg azacitidine tablets as a control; Group 2-12% wt. gain coated tablets; and Group 3-15% wt. gained coated tablets, orally through gavage tube into the stomach, once a day. Blood samples (˜0.8 mL) were collected pre-dose, and 0.25, 0.5, 1, 1.5, 2, 3, 4, 6, 8 hours after dosing for day 1 and 2. The collected blood samples were evaluated for PK data. The PK data for azacitidine is presented in Tables 13-1 and 13-2 and FIG. 12.

TABLE 13-1
Azacitidine AUC_{0-inf}

| | | | | |
|---|---|---|---|---|
| Treatment | | AUCinf | | |
| group | % Coating | (ng*hr/mL) | SD | CV % |

Day 1

| | | | | |
|---|---|---|---|---|
| Uncoated | 0 | 118.8 | 38.7 | 33 |
| Coated FDC-A | 12 | 134.1 | 95.0 | 71 |
| Coated FDC-B | 15 | NA | NA | NA |

Day 2

| | | | | |
|---|---|---|---|---|
| Uncoated | 0 | 417.9 | 237.3 | 57 |
| Coated FDC-A | 12 | 147.5 | NA | NA |
| Coated FDC-B | 15 | NA | NA | NA |

TABLE 13-2
Azacitidine Cₘₐₓ data

| | | | | |
|---|---|---|---|---|
| Treatment group | % Coating | Cₘₐₓ | SD | % CV |

Day 1

| | | | | |
|---|---|---|---|---|
| Uncoated | 0 | 90.5 | 14.8 | 16 |
| Coated FDC-A | 12 | 50.4 | 44.6 | 88 |
| Coated FDC-B | 15 | 4.01 | NA (n = 1) | NA |

Day 2

| | | | | |
|---|---|---|---|---|
| Uncoated | 0 | 202.8 | 97.0 | 48 |
| Coated FDC-A | 12 | 54.3 | 72.4 | 133 |
| Coated FDC-B | 15 | 118 | NA (n = 2) | NA |

The data shows that Surelease® (ethylcellulose) coating with pore former can be used to modify the release profile of azacitidine. As shown in FIG. 12, coated tablets with 12% and 15% wt. gain resulted in a lag time of 30 mins to 1.5 hrs, respectively. The release of the drug was extended to 4-5 hrs for the 12-15% coated tablets vs. 30 minutes for the uncoated tablets. 12% and 15% coated tablets underperformed in PK studies in comparison to uncoated tablets. As shown in Tables 13-1 and 13-2, the 15% wt. gain tablets behaved worse than 12%. The drop in AUC and Cₘₐₓ may be the result of longer lag time and extended release of 4-5 hrs for the 12% and 15% coated tablets.

### Example 14: Pharmacokinetics (PK) Study in Monkeys Using Combination of Example 2

Two different doses of azacitidine 20 mg and 30 mg were tested in monkeys to evaluate pharmacokinetics (PK), where 10 mg is delivered as an immediate release and the rest of the azacitidine dose is delivered as a delayed release.

PK data in monkeys was collected on 12% weight gain coated minitablets at two different dose levels for azacitidine—20 mg and 30 mg, along with uncoated cedazuridine minitablet. Either 50% or 33% of the total azacitidine dose was given as an immediate release and remaining dose was given as a delayed release. Control group 1 contained both azacitidine and cedazuridine uncoated at 20 mg dose. Dosing Groups for the PK study were as follows:

Group 1 (Control): 20 mg cedazuridine, immediate release+20 mg azacitidine, immediate release. Group 2: 20 mg cedazuridine, immediate release+10 mg azacitidine, immediate release+10 mg azacitidine, modified release. Group 3: 20 mg cedazuridine, immediate release+10 mg azacitidine, immediate release+20 mg azacitidine, modified release. Table 14-1 shows the dosing protocol.

TABLE 14-1
Dosing groups for PK study

| | Group 1 | Group 2 | | Group 3 | |
|---|---|---|---|---|---|
| | uncoated | Cedazuridine: | | Cedazuridine: | |
| | cedazuridine | Immediate Release | | Immediate Release | |
| | and azacitidine | Azacitidine: 50% dose | | Azacitidine: 33% dose | |
| | fixed dosage | immediate release (Uncoated) + | | immediate release (Uncoated) + | |
| | immediate | 50% dose modified release | | 67% dose modified release | |
| Description | release tablet | (12% wt. gain, Coated) | | (12% wt. gain, Coated) | |
| Component | Uncoated FDC | Cedazuridine | Azacitidine | Cedazuridine | Azacitidine |
| | tablet | capsule | capsule | capsule | capsule |
| Unit Dose | 1 uncoated | One (1) | One (1) white, | One (1) | One (1) |
| | FDC tablet | orange, size 0, | size 0, VCAPS | orange, size 0, | white, size 0, |
| | containing both | VCAPS Plus | Plus capsule | VCAPS Plus | VCAPS Plus |
| | azacitidine and | capsule | containing two | capsule | capsule |
| | cedazuridine | containing one | (2) uncoated | containing one | containing |
| | | (1) uncoated | and two (2) | (1) uncoated | two (2) |
| | | 20 mg | coated, 5 mg | 20 mg | uncoated and |
| | | cedazuridine | azacitidine | cedazuridine | four (4) |
| | | tablet | minitablets | tablet | coated, 5 mg |
| | | | | | azacitidine |
| | | | | | minitablets |
| Cedazuridine | 20 | 20 | — | 20 | — |
| dose (mg) | | | | | |
| Azacitidine | 20 | — | 20 | — | 30 |
| dose (mg) | | | | | |

Tables 14-2 and 14-3 provide data from this study on Day 1 and Day 2.

TABLE 14-2
Azacitidine AUC_{0-inf}

| | | | |
|---|---|---|---|
| | AUCinf | | |
| Treatment group | (ng*hr/mL) | SD | CV % |

Day 1

| | | | |
|---|---|---|---|
| Group 1 (Control): 20 mg uncoated azacitidine + cedazuridine | 74 | 49 | 67 |
| Group 2: 20 mg uncoated cedazuridine + 10 mg uncoated | 31 | NA (n = 2) | NA |
| azacitidine + 10 mg coated azacitidine | | | |
| Group 3: 20 mg uncoated cedazuridine + 10 mg uncoated | 64 (N = 3) | 31 | 49 |
| azacitidine + 20 mg coated azacitidine | 117* | 157* | 134%* |

Day 2

| | | | |
|---|---|---|---|
| Group 1 (Control): 20 mg uncoated azacitidine + cedazuridine | 238 | 142 | 59 |
| Group 2: 20 mg uncoated cedazuridine + 10 mg uncoated | 132 | 75 | 57 |
| azacitidine + 10 mg coated azacitidine | | | |
| Group 3: 20 mg uncoated cedazuridine + 10 mg uncoated | 304 (N = 3) | 70 | 23 |
| azacitidine + 20 mg coated azacitidine | 478* | 352* | 74* |

*Outlier included

TABLE 14-3
Azacitidine Cₘₐₓ data

| | | | |
|---|---|---|---|
| Treatment group | Cₘₐₓ | SD | CV % |

Day 1

| | | | |
|---|---|---|---|
| Group 1 (Control): 20 mg uncoated | 43 | 25 | 58 |
| azacitidine + cedazuridine | | | |
| Group 2: 20 mg uncoated cedazuridine + | 18.8 | NA | NA |
| 10 mg uncoated azacitidine + 10 | | | |
| mg coated azacitidine | | | |
| Group 3: 20 mg uncoated cedazuridine + | 23.8 (N = 3) | 21 | 89 |
| 10 mg uncoated azacitidine + 20 | 96* | 145* | 151* |
| mg coated azacitidine | | | |

Day 2

| | | | |
|---|---|---|---|
| Group 1 (Control): 20 mg uncoated | 144 | 79 | 55 |
| azacitidine + cedazuridine | | | |
| Group 2: 20 mg uncoated cedazuridine + | 63.3 | 37 | 58 |
| 10 mg uncoated azacitidine + 10 | | | |
| mg coated azacitidine | | | |
| Group 3: 20 mg uncoated cedazuridine + | 144 (N = 3) | 10 | 7 |
| 10 mg uncoated azacitidine + 20 | 223* | 157* | 70* |
| mg coated azacitidine | | | |

*one outlier included

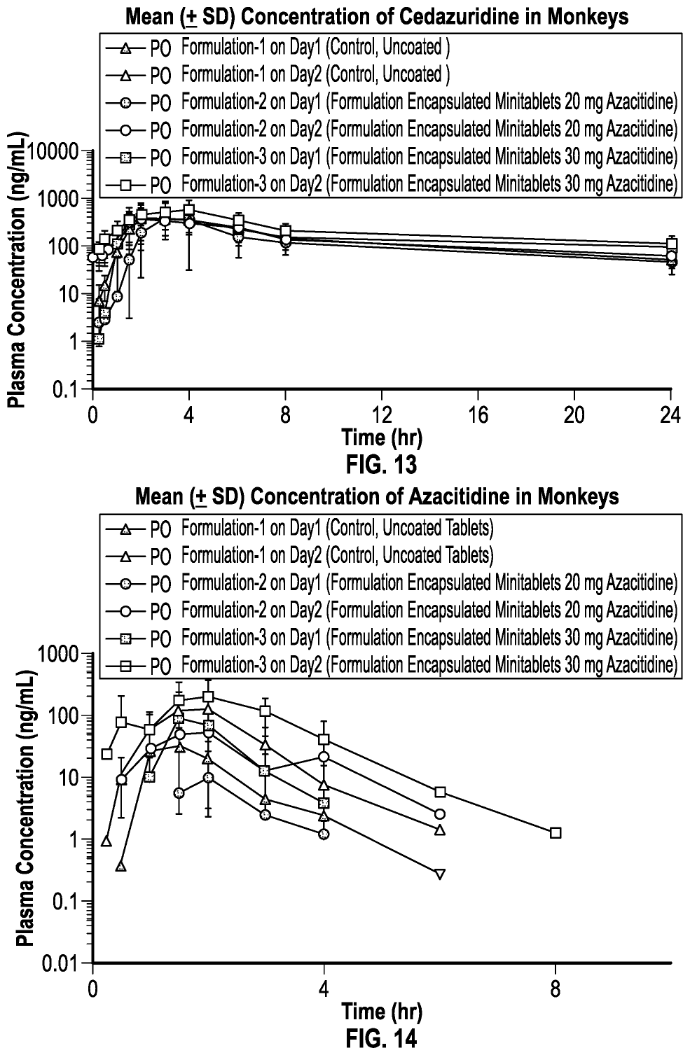

FIG. 13 shows cedazuridine mean concentration time profile for the three groups on Day 1 and Day 2. FIG. 14 shows azacitidine mean concentration time profile for the three groups on Day 1 and Day 2. Azacitidine round minitablets containing 5 mg azacitidine coated with 12% wt. gain Surelease®: Pore former (70:30) extended the release of the drug to 90 mins. Delivering cedazuridine as an immediate release and azacitidine as a combination of immediate release and modified release improved the bioavailability and exposure. On Day 2, the plasma concentration of Group 2 where 20 mg azacitidine dose delivered as 50% immediate release and 50% modified release was slightly lower in comparison to the azacitidine plasma concentration of azacitidine in a control group. However, the plasma concentration of Group 3, 30 mg azacitidine dose where 33% of the azacitidine dose was delivered as an immediate release and 67% of the azacitidine dose was delivered as a modified release had higher plasma concentration in comparison to the control group.

### Example 15: Pharmacokinetics (PK) Study in Monkeys Using Combination of Example 3

In this study, azacitidine was delivered only as a delayed release at three different dose levels 20 mg, 32 mg and 40 mg and two (2) different coating weight gains (15%, 20%). Cedazuridine dose remained constant in all the groups at 20 mg and delivered as an immediate release as described in Example 3. Azacitidine was delivered in the form of minitablets in a HPMC capsule for this round of PK study. Each minitablet contained 4 mg of azacitidine described in Example 3. The PK study was divided in two parts to accommodate 3 different azacitidine doses and two different coating weight gains.

### Dosing Groups for PK Study

The configuration of different dosing groups for the PK study are summarized below and in Table 15-1. The PK study was divided in two parts based on the drug release data for two different coating weight gains. Azacitidine dose was increased incrementally from 20 mg to 40 mg between the groups. Cedazuridine was delivered at 20 mg dose as an immediate release.

### Part 1 of PK Studies, Total 4 Groups (Faster Release)

Group 1 (Control): 20 mg Cedazuridine, immediate release+20 mg Azacitidine, immediate release. Group 2: 20 mg Cedazuridine, immediate release+20 mg Azacitidine, 80/20 coated at 15% wt. gain. Group 3: 20 mg Cedazuridine, immediate release+32 mg Azacitidine, 80/20 coated at 15% wt. gain. Group 4: 20 mg Cedazuridine, immediate release+40 mg Azacitidine, 80/20 coated at 15% wt. gain.

### Part 2 of PK Studies, Total 4 Groups (Slower Release)

Group 1 (Control): 20 mg Cedazuridine, immediate release+20 mg Azacitidine, immediate release. Group 5: 20 mg Cedazuridine, immediate release+20 mg Azacitidine, 75/25 coated at 20% wt. gain. Group 6: 20 mg Cedazuridine, immediate release+32 mg Azacitidine, 75/25 coated at 20% wt. gain. Group 7: 20 mg Cedazuridine, immediate release+40 mg Azacitidine, 75/25 coated at 20% wt. gain.

TABLE 15-1
Dosing Group for Part 1 and Part 2 PK studies

| | | Parameters | | | | | | |
|---|---|---|---|---|---|---|---|---|
| | | Control | Faster release (Part 1) | | | Slower release (Part 2) | | |
| | | Group No. | | | | | | |
| | | Group 1 | Group 2 | Group 3 | Group 4 | Group 5 | Group 6 | Group 7 |
| Description | | FDC | Size 1 Vcaps Plus White capsule shell containing | | | | | |
| | | tablets, | cedazuridine uncoated round tablet/coated | | | | | |
| | | oval, | azacitidine minitablets | | | | | |
| | | uncoated | | | | | | |
| Coating | Surelease/Pore | NA | 80/20 | | | 75/25 | | |
| | former | | | | | | | |
| | Weight gain for | NA | 15% | | | 20% | | |
| | coated azacitidine | | | | | | | |
| | minitablets | | | | | | | |
| Strength | cedazuridine | 20 mg | | | | | | |
| | Azacitidine | 20 mg | 20 mg | 32 mg | 40 mg | 20 mg | 32 mg | 40 mg |
| Qty of | cedazuridine, 20 | NA | 1 | 1 | 1 | 1 | 1 | 1 |
| minitablets | mg, round, | | | | | | | |
| per capsule | uncoated | | | | | | | |
| | Azacitidine, 4 mg, | NA | 5 | 8 | 10 | 5 | 8 | 10 |
| | coated minitablets | | | | | | | |

### Part 1 PK Study

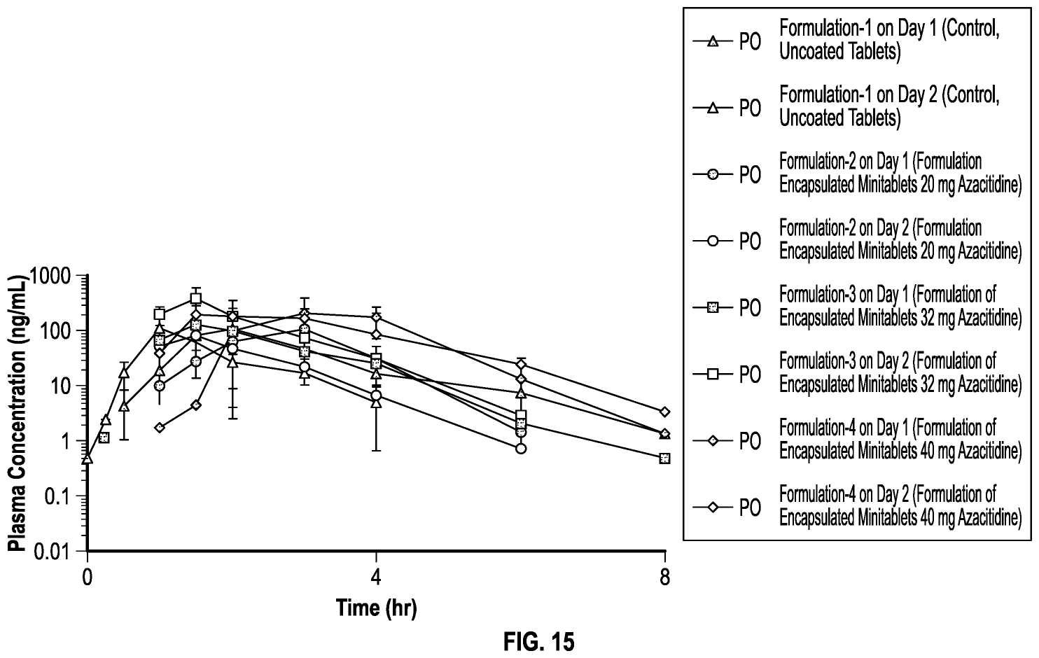

The PK data in monkeys was collected on 15% and 20% coated minitablets at three different dose levels for azacitidine 20 mg, 32 mg, and 40 mg along with uncoated cedazuridine tablet. Control group 1 contained both azacitidine and cedazuridine uncoated at 20 mg dose. Part 1 of the PK study was performed with 15% wt. gain coated minitablets. The PK data for Part 1 of the study is in Tables 15-2 and 15-3 and FIG. 15. Based on the PK data gathered for Part 1 of the study, the average exposure for Group 2, was equivalent to control Group 1 with the same dose of azacitidine, however, 2 out 3 animals showed much lower exposures. Higher dose of 30 mg and 40 mg helped compensate for the loss of absorption with the coated minitablets.

TABLE 15-2
Azacitidine AUC_{0-inf} for 15% wt. gain coated
minitablets at 80/20 ratio (Part 1 PK study)

| | AUC_{inf} | | |
|---|---|---|---|
| Treatment group | (ng*hr/mL) | SD | CV % |
| | Day 1, Azacitidine | | |
| Group 1 (Control): 20 mg uncoated | 232 | 90 | 39 |
| azacitidine + cedazuridine | | | |
| Group 2: 20 mg cedazuridine, | 213 | 255 | 120 |
| immediate release + 20 mg azacitidine, | | | |
| 80/20 coated at 15% wt. gain | | | |
| Group 3: 20 mg cedazuridine, | 273 | 147 | 54 |
| immediate release + 32 mg azacitidine, | | | |
| 80/20 coated at 15% wt. gain | | | |
| Group 4: 20 mg cedazuridine, | 595 | 435 | 73 |
| immediate release + 40 mg azacitidine, | | | |
| 80/20 coated at 15% wt. gain | | | |
| | Day 2, Azacitidine | | |
| Group 1 (Control): 20 mg uncoated | 145 | 82 | 56 |
| azacitidine + cedazuridine | | | |
| Group 2: 20 mg cedazuridine, | 142 | 122 | 86 |
| immediate release + 20 mg azacitidine, | | | |
| 80/20 coated at 15% wt. gain | | | |
| Group 3: 20 mg cedazuridine, | 568 | 311 | 55 |
| immediate release + 32 mg azacitidine, | | | |
| 80/20 coated at 15% wt. gain | | | |
| Group 4: 20 mg cedazuridine, | 647 | 700 | 108 |
| immediate release + 40 mg azacitidine, | | | |
| 80/20 coated at 15% wt. gain | | | |

TABLE 15-3
Azacitidine Cₘₐₓ data for 15% wt. gain coated
minitablets at 80/20 ratio (Part 1 PK study)

| Treatment group | Cₘₐₓ | SD | CV % |
|---|---|---|---|
| | Day 1, Azacitidine | | |
| Group 1 (Control): 20 mg uncoated azacitidine + | 114 | 48 | 42 |
| cedazuridine | | | |
| Group 2: 20 mg Cedazuridine, immediate | 120 | 131 | 109 |
| release + 20 mg Azacitidine, 80/20 coated at | | | |
| 15% wt. gain | | | |
| Group 3: 20 mg Cedazuridine, immediate | 146 | 49 | 33 |
| release + 32 mg Azacitidine, 80/20 coated at | | | |
| 15% wt. gain | | | |
| Group 4: 20 mg Cedazuridine, immediate | 219 | 167 | 76 |
| release + 40 mg Azacitidine, 80/20 coated at | | | |
| 15% wt. gain | | | |
| | Day 2, Azacitidine | | |
| Group 1 (Control): 20 mg uncoated azacitidine + | 132 | 99 | 75 |
| cedazuridine | | | |
| Group 2: 20 mg Cedazuridine, immediate | 85 | 58 | 69 |
| release + 20 mg Azacitidine, 80/20 coated at | | | |
| 15% wt. gain | | | |
| Group 3: 20 mg Cedazuridine, immediate | 358 | 241 | 67 |
| release + 32 mg Azacitidine, 80/20 coated at | | | |
| 15% wt. gain | | | |
| Group 4: 20 mg Cedazuridine, immediate | 247 | 165 | 67 |
| release + 40 mg Azacitidine, 80/20 coated at | | | |
| 15% wt. gain | | | |

### Part 2 PK Study

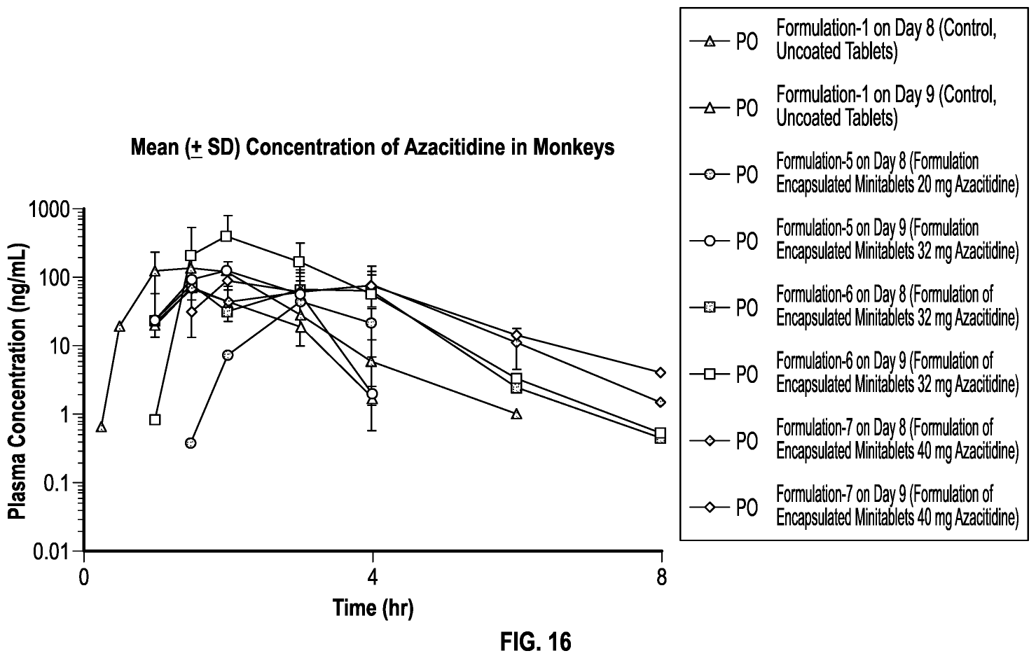

20% wt. gain coated minitablets were used in Part 2 of the PK study. The PK data for part 2 of the study is in Tables 15-4 and 15-5. FIG. 16 shows azacitidine mean concentration profile for 20% wt. gain coated minitablets at 75/25 ratio. For Group 5, where the same dose of azacitidine 20 mg was given in a delayed release manner, AUC_{0-inf} on Day 2 decreased from 265 ng*hr/mL to 229 ng*hr/mL. However, Groups 6 and 7 outperformed control group 1 and exposure levels were either similar or higher to control group on Day 2.

TABLE 15-4
Azacitidine AUC_{0-inf} for 20% wt. gain coated
minitablets at 75/25 ratio (Part 2 PK study)

| | AUC_{inf} | | |
|---|---|---|---|
| Treatment group | (ng*hr/mL) | SD | CV % |
| | Day 1, Azacitidine | | |
| Group 1 (Control): 20 mg uncoated | 108 | 39 | 36 |
| azacitidine + cedazuridine | | | |
| Group 5: 20 mg cedazuridine, | 66 | 55 | 83 |
| immediate release + 20 mg azacitidine, | | | |
| 75/25 coated at 20% wt. gain | | | |
| Group 6: 20 mg cedazuridine, | 248 | 124 | 50 |
| immediate release + 32 mg azacitidine, | | | |
| 75/25 coated at 20% wt. gain | | | |
| Group 7: 20 mg cedazuridine, | 274 | 121 | 44 |
| immediate release + 40 mg azacitidine, | | | |
| 75/25 coated at 20% wt. gain | | | |
| | Day 2, Azacitidine | | |
| Group 1 (Control): 20 mg uncoated | 265 | 128 | 48 |
| azacitidine + cedazuridine | | | |
| Group 5: 20 mg cedazuridine, | 229 | 84 | 37 |
| immediate release + 20 mg azacitidine, | | | |
| 75/25 coated at 20% wt. gain | | | |
| Group 6: 20 mg cedazuridine, | 648 | 593 | 92 |
| immediate release + 32 mg azacitidine, | | | |
| 75/25 coated at 20% wt. gain | | | |
| Group 7: 20 mg cedazuridine, | 302 | 116 | 38 |
| immediate release + 40 mg azacitidine, | | | |
| 75/25 coated at 20% wt. gain | | | |

TABLE 15-5
Azacitidine Cₘₐₓ data for 20% wt. gain coated
minitablets at 75/25 ratio (Part 2 PK study)

| Treatment group | Cₘₐₓ | SD | CV % |
|---|---|---|---|
| | Day 1, Azacitidine | | |
| Group 1 (Control): 20 mg uncoated | 66 | 28 | 43 |
| azacitidine + cedazuridine | | | |
| Group 5: 20 mg cedazuridine, | 48 | 47 | 97 |
| immediate release + 20 mg azacitidine, | | | |
| 75/25 coated at 20% wt. gain | | | |
| Group 6: 20 mg cedazuridine, | 143 | 88 | 62 |
| immediate release + 32 mg azacitidine, | | | |
| 75/25 coated at 20% wt. gain | | | |
| Group 7: 20 mg cedazuridine, | 111 | 29 | 26 |
| immediate release + 40 mg azacitidine, | | | |
| 75/25 coated at 20% wt. gain | | | |
| | Day 2, Azacitidine | | |
| Group 1 (Control): 20 mg uncoated | 156 | 91 | 58 |
| azacitidine + cedazuridine | | | |
| Group 5: 20 mg cedazuridine, | 141 | 54 | 38 |
| immediate release + 20 mg azacitidine, | | | |
| 75/25 coated at 20% wt. gain | | | |
| Group 6: 20 mg cedazuridine, | 404 | 432 | 107 |
| immediate release + 32 mg azacitidine, | | | |
| 75/25 coated at 20% wt. gain | | | |
| Group 7: 20 mg cedazuridine, | 135 | 26 | 19 |
| immediate release + 40 mg azacitidine, | | | |
| 75/25 coated at 20% wt. gain | | | |

As shown in FIGS. 15 and 16, azacitidine 20 mg minitablets coated with Surelease: Pore former (80:20) at 15% wt. gain extended the release of azacitidine to 90 mins and minitablets coated with Surelease: Pore former (75:25) at 20% wt. gain extended the release of azacitidine to 120 mins.

As shown in Tables 15-2 and 15-4, Based on the Day 2 azacitidine data, the AUC₀₋ₗₐₛₜ from Group 2 and 5, 20 mg azacitidine dose delivered as a modified release was slightly lower in comparison to the azacitidine AUC in the control group, where azacitidine was delivered as an immediate release. However, for all the other groups, Groups 3 and 6 (32 mg dose) and Groups 4 and 7 (40 mg dose), azacitidine exposure was higher in comparison to AUC from the control group.

### Example 16: Pharmacokinetics (PK) Study in Monkeys Using Combination of Example 4

In this study, azacitidine was delivered as a delayed release at two different dose levels 20 mg and 40 mg at 5.3% enteric coating weight gain. Cedazuridine dose remained constant in all the groups at 20 mg and delivered as an immediate release. Azacitidine was delivered in the form of minitablets in a HPMC capsule for PK study. Each minitablet contained 4 mg of azacitidine. Azacitidine minitablets were coated with Eudragit® enteric coating, which is a 3-layer enteric coating that prevents the release of the drug in acid phase (pH 1.2) followed by rapid release in buffer phase at pH 6.8. The inner most layer is a seal coat followed by intermediate coat containing an alkaline agent and a final outer enteric coat with Eudragit® polymer. Eudragit® L30-D55 polymer was used to provide an enteric coat on azacitidine minitablets.

### PK Study

PK Dosing Groups used in this study were as follows: Group 1 (Control): 20 mg Cedazuridine, immediate release+20 mg Azacitidine, immediate release; Group 2: 20 mg Cedazuridine, immediate release+20 mg Azacitidine, 5.3% Eudragit® coating; Group 3: 20 mg Cedazuridine, immediate release+40 mg Azacitidine, 5.3% Eudragit® coating.

The PK data in monkeys was collected on 5.3% coated minitablets at two different dose levels for azacitidine 20 mg, and 40 mg along with uncoated cedazuridine tablet. Control group 1 contained both azacitidine and cedazuridine uncoated at 20 mg dose. The dosing groups are listed in Table 16-1.

TABLE 16-1
Dosing Groups for Eudragit ® coated minitablets

| | | Parameters | | |
|---|---|---|---|---|
| | | Control | Groups | |
| | | Group No. | | |
| | | | Group 2 | Group 3 |
| | | | Size 1 Vcaps Plus White capsule | |
| | | Group 1 | shell containing cedazuridine | |
| | | FDC tablets, | round tablet + coated azacitidine | |
| Description | | oval, uncoated | minitablets | |
| | Coating | NA | Eudragit ® | |
| | Weight gain | NA | 5.3% | |
| Strength | cedazuridine | 20 mg | | |
| | Azacitidine | 20 mg | 20 mg | 40 mg |
| Qty of | cedazuridine, 20 mg, | NA | 1 | 1 |
| minitablets | round, uncoated | | | |
| per | azacitidine, 4 mg, | NA | 5 | 10 |
| capsule | coated minitablets | | | |

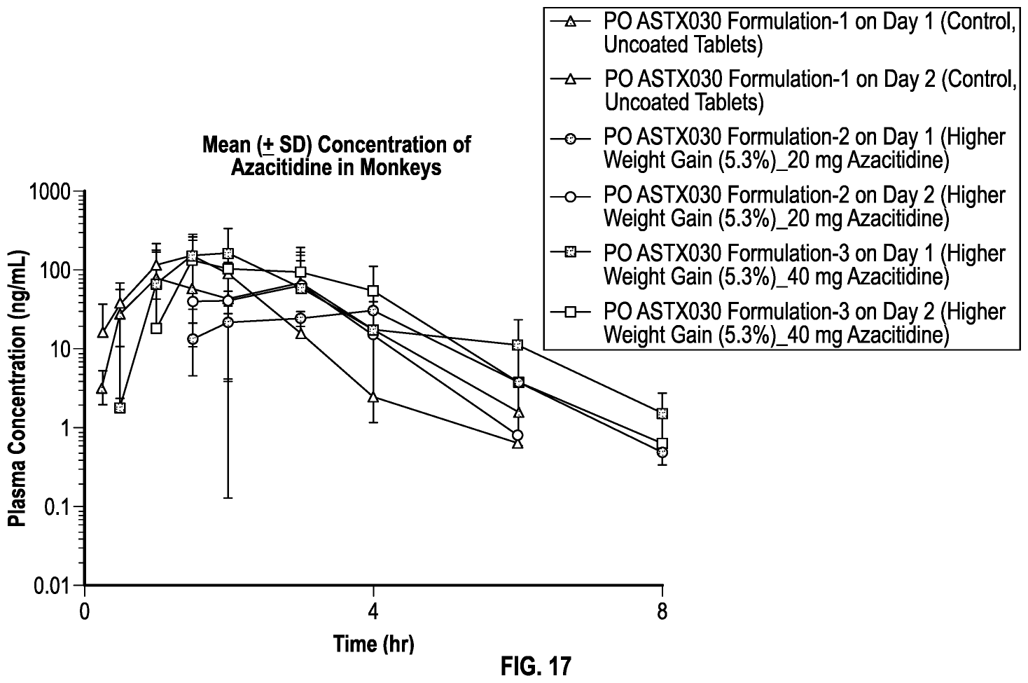

The PK data for 5.3% polymer wt. gain Eudragit (coated minitablets is presented in FIG. 17 and tabulated in Tables 16-2 and 16-3. Five (5) minitablets or ten minitablets (10) were placed in a Size 1 HPMC based capsule (Vcaps Plus) for the PK study. The exposure level for azacitidine from Group 2 on Day 2 was slightly lower in comparison to the control group. However, exposure level for higher dose (40 mg dose) was higher in comparison to the control group. The higher azacitidine dose compensated the loss in the exposure caused by delayed release.

TABLE 16-2
Azacitidine AUC₀₋ₗₐₛₜ

| | Day 1 | | | Day 2 | | |
|---|---|---|---|---|---|---|
| | AUCinf | | | AUCinf | | |
| Treatment Group | (ng*hr/mL) | SD | CV % | (ng*hr/mL) | SD | CV % |
| 1-Uncoated FDC (20 + 20 mg) | 201 | 82 | 41 | 240 | 200 | 84 |
| 2-Encaps. Minitabs (20 mg | 121 | 47 | 38 | 222 | 207 | 93 |
| uncoated cedazuridine + 20 mg | | | | | | |
| coated azacitidine, 5.3% wt. gain) | | | | | | |
| 3-Encaps. Minitabs (20 mg | 354 | 243 | 69 | 341 | 235 | 69 |
| uncoated cedazuridine + 40 mg | | | | | | |
| coated azacitidine, 5.3% wt. gain) | | | | | | |

TABLE 16-3
Azacitidine Cₘₐₓ

| | Day 1 | | | Day 2 | | |
|---|---|---|---|---|---|---|
| | Cmax | | | Cmax | | |
| Treatment Group | (ng/mL) | SD | % CV | (ng/mL) | SD | % CV |
| 1-Uncoated FDC (20 + 20 mg) | 133 | 69 | 52 | 158 | 117 | 74 |
| 2-Encaps. Minitabs (20 mg | 47 | 25 | 53 | 107 | 99 | 93 |
| uncoated cedazuridine + 20 mg | | | | | | |
| coated azacitidine, 5.3% wt. gain) | | | | | | |
| 3-Encaps. Minitabs (20 mg | 202 | 155 | 77 | 156 | 75 | 48 |
| uncoated cedazuridine + 40 mg | | | | | | |
| coated azacitidine, 5.3% wt. gain) | | | | | | |

Delivering cedazuridine as an immediate release component in a fixed dose combination improved azacitidine bioavailability and exposure. Based on the Day 2 azacitidine data, the exposure from Group 2, 20 mg azacitidine dose delivered as a delayed release was slightly lower in comparison to the azacitidine exposure from the control group, where azacitidine was delivered as an immediate release. However, for Group 3 (40 mg dose), azacitidine exposure was higher in comparison to the control group. pH sensitive coated azacitidine with uncoated cedazuridine in an immediate release capsule achieved exposures similar to uncoated azacitidine but may require a higher dose.

### Example 17: Pharmacokinetics (PK) Study in Monkeys Using Combination of Example 6

For this study, 20 mg azacitidine was delivered as a delayed release at 4.0% enteric coating weight gain, as described in Example 6. Cedazuridine dose remained constant in all the groups at 20 mg and delivered as an immediate release. Azacitidine was delivered in the form of minitablets in a HPMC capsule for PK study. Each minitablet contained 4 mg of azacitidine as described in Example 6. The inner most layer is a seal coat followed by a final outer enteric coat with an Eudragit® polymer. No intermediate coating was applied on the azacitidine minitablets for this round of PK study. Eudragit® L30-D55 polymer was used to provide an enteric coat on azacitidine minitablets.

### PK Study

PK Dosing Groups used in this study were as follows: Group 1 (Control): 20 mg Cedazuridine, immediate release+20 mg Azacitidine, immediate release; Group 2: 20 mg Cedazuridine, immediate release+20 mg Azacitidine, 4.0% Eudragit® coating.

Total four monkeys were dosed with Group 2 composition (20 mg Cedazuridine, immediate release+20 mg Azacitidine, 4.0% Eudragit® coating) on days 1, 2; and Group 1 composition (Control, 20 mg Cedazuridine, immediate release+20 mg Azacitidine, immediate release) on days 8, 9, orally through gavage tube into the stomach, once a day. Blood samples (˜0.8 mL) were collected pre-dose, and 0.25, 0.5, 1, 1.5, 2, 3, 4, 6, 8 hours after dosing for days 1, 8 (first dosing days) and pre-dose, and 0.25, 0.5, 1, 1.5, 2, 3, 4, 6, 8, and 24 hours after dosing for days 2, 9 (second dosing days). The collected blood samples were evaluated for PK data.

TABLE 17-1
Dosing Group for PK Study with 4.0% Eudragit ® L30-
D-55 coating

| | Cedazuridine Immediate | |
|---|---|---|
| Group | Release | Azacitidine |
| 1 (Control) | 20 mg | 20 mg (uncoated) |
| 2 | 20 mg | 20 mg (coated) |

The PK data for 4.0% polymer wt. gain Eudragit®3 coated azacitidine minitablets is presented in Table 17-2. As shown in Table 17-2, coated azacitidine minitablets delivers higher AUC and Cₘₐₓ at the same dose, due to delayed absorption of azacitidine while cedazuridine absorption is not delayed.

TABLE 17-2
PK data for Study with 4.0% Eudragit ® L30-D-55 coating

| | Cₘₐₓ | | AUC | |
|---|---|---|---|---|
| Treatment Group | (ng*hr/mL) | SD | (ng*hr/mL) | SD |
| 1-Uncoated FDC | 160 | 119 | 301 | 253 |
| (20 + 20 mg) | | | | |
| 2-20 mg uncoated | 258 | 157 | 554 | 386 |
| cedazuridine + | | | | |
| 20 mg coated | | | | |
| azacitidine | | | | |
| 4.0% wt. gain) | | | | |

### Example 18: Dissolution Study of FDC Capsule

Six capsule samples of Capsule 1 and six samples of Capsule 2 (as described below) were submitted for dissolution testing per USP <711> dissolution-Method A for delayed release dosage form.

TABLE 18-1

| | Azacitidine 20 mg/Cedazuridine | | | Azacitidine 20 mg/Cedazuridine | | |
|---|---|---|---|---|---|---|
| | 20 mg in size 00 | | | 20 mg in size 00 | | |
| | Capsule 1 (No buffer), 62% fill | | | Capsule 2 (with buffer), 50% fill | | |
| | capacity | | | capacity | | |
| | | | Qty. for | | | Qty. for |
| | | | 10 g blend | | | 8 g blend |
| Ingredients | % | mg/capsule | (mg) | % | mg/capsule | (mg) |
| Cedazuridine | 8.0 | 20.0 | 800.0 | 10.0 | 20.0 | 800.0 |
| Azacitidine | 8.0 | 20.0 | 800.0 | 10.0 | 20.0 | 800.0 |
| Lactose | 35.8 | 89.4 | 3574.6 | 26.2 | 52.4 | 2092.3 |
| monohydrate | | | | | | |
| MCC (Avicel ® | 22.2 | 55.6 | 2224.4 | 16.3 | 32.6 | 1302.0 |
| PH-102) | | | | | | |
| MCC (Avicel ® | 12.8 | 32.0 | 1281.0 | 9.4 | 18.8 | 749.8 |
| PH-200) | | | | | | |
| Sodium | 0.0 | 0.0 | 0.0 | 15.0 | 30.0 | 1200.0 |
| Phosphate | | | | | | |
| anhydrous | | | | | | |
| Croscarmellose | 8.0 | 20.0 | 800.0 | 8.0 | 16.0 | 640.0 |
| Sodium | | | | | | |
| Hypromellose | 2.0 | 5.0 | 200.0 | 2.0 | 4.0 | 160.0 |
| Colloidal | 2.1 | 5.4 | 214.0 | 2.1 | 4.2 | 168.0 |
| Silicon | | | | | | |
| Dioxide | | | | | | |
| Magnesium | 1.1 | 2.7 | 106.0 | 1.1 | 2.2 | 88.0 |
| Stearate | | | | | | |
| Total | 100.0 | 250.0 | 10000.0 | 100.0 | 200.2 | 8000.0 |

As the dissolution of enteric capsule is pH-dependent, a two-stage dissolution was performed, with acid stage followed by buffer stage. The dissolution method used a USP type 1 basket apparatus operated at 75 rpm. The dissolution medium is 500 mL of 0.1 N HCl maintained at 37° C. The capsule samples were enclosed with spiral sinker CAPWHT-XS and dropped into the dissolution vessel. The duration of the acid stage is 2 hours, with dissolution samples pulled at 30, 60, 90 and 120 min for analysis. At the end of the acid stage, the dissolution media was switched to 50 mM phosphate buffer at pH 6.8, pre-heated at 37° C. The dissolution in buffer stage continues for another 90 min, with dissolution samples pulled at 135, 150, 165, 180, 210 min time points for analysis. Infinity spin was also performed at 250 rpm for additional 15 min. All the dissolution testing was performed in USP apparatus 1. At the last time point, which is referred to as infinity, the rotation of the basket increased from 75 rpm to 250 rpm in the dissolution media. The amount of cedazuridine and azacitidine released was determined by a reversed-phase HPLC method, comparing the response of the dissolution sample with that of the reference standard (purchased from Shilpa Chemicals).

The HPLC conditions were as follows. Mobile Phase A (M.P.A.): 10 mM sodium phosphate buffer in water, pH 6.8. Mobile Phase B (M.P.B.): 50/50 (v/v) 10 mM sodium phosphate buffer in water, pH 6.8/ACN. Column: X select CSH phenyl-hexyl 2.5 um, 4.6×75 mm, P/N 186006134. HPLC system: waters Alliance system. Diluent: 0.1N HCl and 50 mM 10 mM sodium phosphate buffer in water, pH 6.8. HPLC condition: column temp: 23 C, inj vol: 10 uL. 12 minute run going from 98:2 M.P.A:M.P.B to 80:20 M.P.A:M.P.B to 98:2 M.P.A:M.P.B, gradient, flow rate 0.7 ml/min.

Due to the instability of azacitidine in dissolution medium across the physiological pH range, the major degradation products were also included in the quantitation of total dissolved azacitidine. Cedazuridine degraded in acid stage. Therefore, major degradation product of cedazuridine, and its epimer, was also included in the quantitation of total dissolved cedazuridine in acid stage. In the buffer stage, cedazuridine degradation is minimal and only the cedazuridine peak is included in the quantitation of cedazuridine. The total dissolution reported includes the total of % dissolved for acid stage and buffer stage.

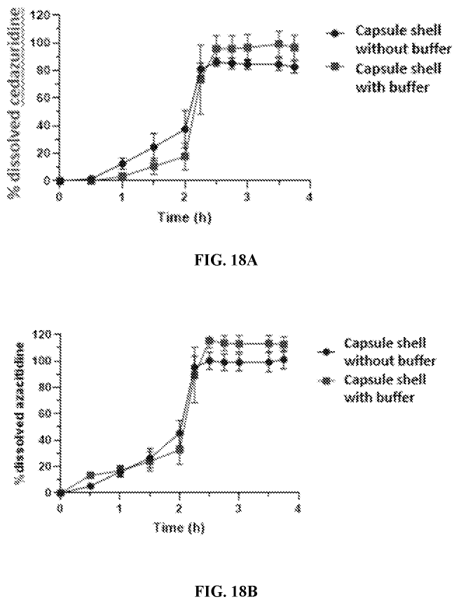

FIG. 18A and FIG. 18B show the dissolution profiles for cedazuridine and azacitidine respectively. As shown in FIGS. 18A and 18B, both azacitidine and cedazuridine were slowly released in acid stage, while were rapidly released at buffer stage.

### Example 19: Bioequivalence of Capsule Formulations in Cynomolgus Monkeys

Male Cynomolgus monkeys of 3-6 kg weight were used. A total of 12 animals was assigned to this study. Animals were dosed with tablet/capsules on both Day 1 and Day 2 as indicated in the table below. The dose level was 20 mg azacitidine and 20 mg cedazuridine per animal. The test tablet (Example 12)/capsule (Example 11) was administered orally.

TABLE 19-1

| Formulation | Reference | Capsule-1 | Capsule-2 |
|---|---|---|---|
| Description | FDC reference tablet: | New Capsule-1 (enteric | New Capsule-2 (enteric |
| | no coating | capsule with no buffer) | capsule with buffer) |
| Component | Azacitidine and | Azacitidine and | Azacitidine and |
| | cedazuridine tablet | cedazuridine capsule | cedazuridine capsule |
| Composition | 20 mg Azacitidine and | 20 mg Azacitidine and | 20 mg azacitidine and 20 |
| | 20 mg cedazuridine | 20 mg cedazuridine per | mg cedazuridine per each |
| | per each FDC tablet | each capsule | capsule |

Blood samples for plasma were collected as follows: pre-dose (0), 0.25, 0.5, 1, 1.5, 2, 3, 4, 6, and 8 hr post Day 1 dose; pre Day 2 dose (24 hr post Day 1 dose), 0.25, 0.5, 1, 1.5, 2, 3, 4, 6, 8 and 24 hr post Day 2 dose. The blood samples were collected via cephalic or appropriate vein at the designed time points. A target of 0.8 mL of blood was collected into K2EDTA-containing tubes that were pre-spiked with 20 L of 0.4 mg/mL (tetrahydrouridine) THU.

Hematology of each animal was evaluated once during acclimation (˜4 days before dosing day) and on Day 9.

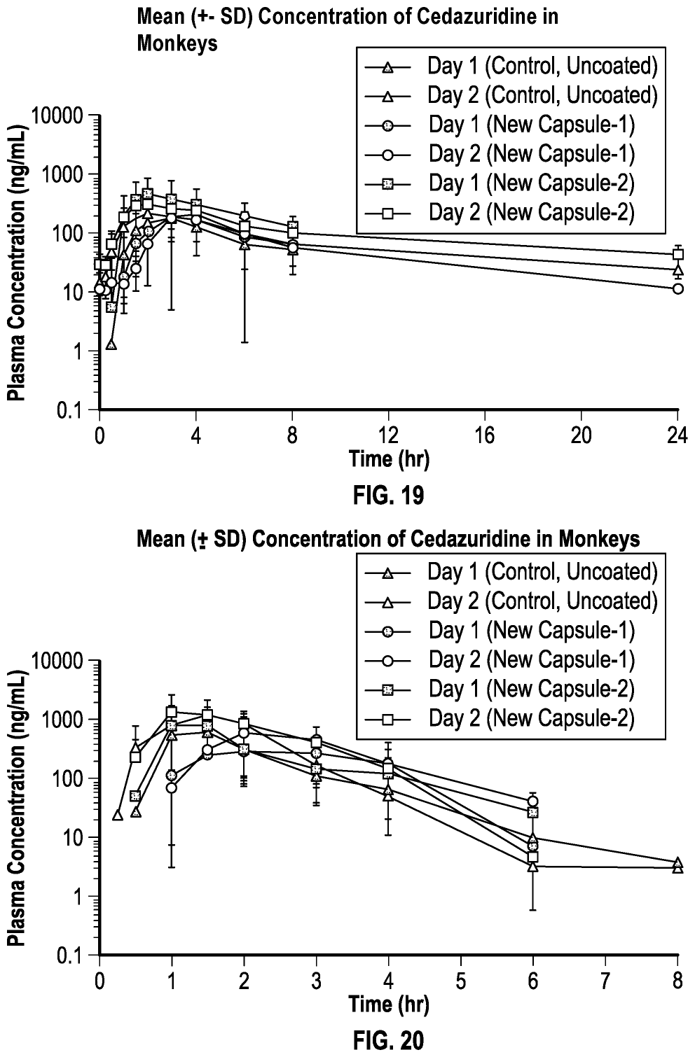

Table 19-2 and Table 19-3 show the data from the experiment above. FIG. 19 shows plasma exposure of cedazuridine in monkeys using the capsules of Example 17. FIG. 20 shows plasma exposure of azacitidine in monkeys using the capsules of Example 17.

TABLE 19-2

| | | | |
|---|---|---|---|
| | AUCₗₐₛₜ | | |
| Treatment Group | (ng*hr/mL) | SD | CV % |

Day 1 - cedazuridine

| | | | |
|---|---|---|---|
| 1 - Uncoated FDC | 739 | 231 | 31 |
| 2 - Enteric Coated FDC Capsule 1 | 821 | 491 | 60 |
| 3 - Enteric Coated FDC Capsule 2 | 1965 | 1525 | 78 |
| (buffer salt) | | | |

Day 2 - cedazuridine

| | | | |
|---|---|---|---|
| 1 - Uncoated FDC | 1778 | 609 | 34 |
| 2 - Enteric Coated FDC Capsule 1 | 1365 | 799 | 59 |
| 3 - Enteric Coated FDC Capsule 2 | 2630 | 283 | 11 |
| (buffer salt) | | | |

TABLE 19-3

| | | | |
|---|---|---|---|
| | AUCₗₐₛₜ | | |
| Treatment Group | (ng*hr/mL) | SD | CV % |

Day 1 - Azacitidine

| | | | |
|---|---|---|---|
| 1 - Uncoated FDC | 139 | 103 | 74 |
| 2 - Enteric Coated FDC Capsule 1 | 119 | 86 | 73 |
| 3 - Enteric Coated FDC Capsule 2 | 199 | 118 | 59 |
| (buffer salt) | | | |

Day 2 - Azacitidine

| | | | |
|---|---|---|---|
| 1 - Uncoated FDC | 281 | 66 | 23 |
| 2 - Enteric Coated FDC Capsule 1 | 185 | 152 | 82 |
| 3 - Enteric Coated FDC Capsule 2 | 351 | 188 | 54 |
| (buffer salt) | | | |

### Example 20: Clinical Data Using Combination of Example 6

For this study, 20 mg azacitidine was delivered as a delayed release at 4.0% enteric coating weight gain, as described in Example 6. Cedazuridine dose remained constant in all the groups at 20 mg and delivered as an immediate release. Azacitidine was delivered in the form of minitablets in a HPMC capsule for PK study. Each minitablet contained 4 mg of azacitidine as described in Example 6. The inner most layer is a seal coat followed by a final outer enteric coat with an Eudragit® polymer. No intermediate coating was applied on the azacitidine minitablets for this round of PK study. Eudragit® L30-D55 polymer was used to provide an enteric coat on azacitidine minitablets.

Human subjects (n=6) were administered with azacitidine PK Dosing Groups used in this study were as follows: Group 1: 60 mg subcutaneous injection of azacitidine, 75 mg/m²; Group 2: 60 mg oral azacitidine (4.0% Eudragit® coated) only, once a day; Group 3: 60 mg uncoated cedazuridine, oral+60 mg azacitidine (4.0% Eudragit® coated), oral, once a day.

Human subjects (n=6) were dosed with Group 2 composition (oral Azacitidine only) on day −3 (prior to day 1) (“C1D-3”); Group 1 composition (subcutaneous) on day 1 (“C1D1”); and Group 3 composition on days 2-7 (“C1D2”-“C1D7”), once a day. Blood samples were collected pre-dose, and 0.5, 1, 1.5, 2, 2.5, 3, 3.5, 4, 5, 7, 9 and 24 hours after dosing. The collected blood samples were evaluated for PK data.

The azacitidine PK data is shown in Table 20-1. As shown in Table 20-1, co-administration with cedazuridine increased Tₘₐₓ, Cₘₐₓ and AUC for azacitidine, indicating that immediate release cedazuridine increases bioavailability of delayed release azacitidine. Further, oral administration of azacitidine and cedazuridine showed more stable, prolonged release of azacitidine compared to subcutaneous administration of azacitidine.

TABLE 20-1

| | Tₘₐₓ | Cₘₐₓ | AUC₀₋₂₄ |
|---|---|---|---|
| Treatment Group | (hr) | (ng/mL) | (ng*hr/mL) |
| Group 1 (azacitidine, SQ) | 0.6 | 384 | 761 |
| Group 2 (azacitidine DR, | 1.0 | 14 | 30 |
| PO alone) | | | |
| Group 3 (azacitidine DR, | 2.0 | 219 | 726 |
| PO with cedazuridine) | | | |

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A pharmaceutical dosage form comprising cedazuridine, or a pharmaceutically acceptable salt thereof, and azacitidine, or a pharmaceutically acceptable salt thereof, wherein at least a portion of the azacitidine is formulated for modified release.

2. The pharmaceutical dosage form of claim 1, wherein the cedazuridine is formulated for immediate release.

3. The pharmaceutical dosage form of claim 1, wherein the portion of the azacitidine that is formulated for modified release is formulated for enteric release.

4. The pharmaceutical dosage form of claim 1, wherein the portion of azacitidine that is formulated for modified release is provided as minitablets comprising an enteric coat.

5. The pharmaceutical dosage form of claim 4, each azacitidine minitablet comprising:
about 20%-60% w/w of azacitidine, wherein the percentage by weight is relative to the total weight of the uncoated azacitidine minitablets; and
one or more pharmaceutically acceptable excipients.

6. The pharmaceutical dosage form of claim 5, wherein the one or more pharmaceutically acceptable excipients is selected from the group consisting of lactose monohydrate, microcrystalline cellulose, hydroxypropyl methylcellulose (HPMC), croscarmellose sodium, silicon dioxide, and magnesium stearate.

7. The pharmaceutical dosage form of claim 4, wherein the enteric coat comprises polymethacrylate or copolymers thereof.

8. The pharmaceutical dosage form of claim 4, wherein the enteric coat is sensitive to pH variations in the intestine.

9. The pharmaceutical dosage form of claim 4, wherein each azacitidine minitablet further comprises a seal coat beneath the enteric coat.

10. The pharmaceutical dosage form of claim 9, wherein the seal coat comprises hydroxypropyl methylcellulose (HPMC).

11. The pharmaceutical dosage form of claim 1, wherein the azacitidine is formulated for modified release and provided as minitablets comprising an enteric coat.

12. The pharmaceutical dosage form of claim 1, wherein substantially all of the azacitidine is configured to be released outside the stomach.

13. The pharmaceutical dosage form of claim 1, wherein the cedazuridine is uncoated and in the form of minitablets, a tablet, powder, blend, granules, or pellets.

14. The pharmaceutical dosage form of claim 13, wherein the uncoated minitablets, a tablet, powder, blend, granules, or pellets comprises about 10%-40% w/w of cedazuridine.

15. A pharmaceutical dosage form comprising cedazuridine, or a pharmaceutically acceptable salt thereof, and azacitidine, or a pharmaceutically acceptable salt thereof, wherein:
the cedazuridine is formulated for immediate release; and
the azacitidine is formulated as modified release minitablets comprising an enteric coat.

16. The pharmaceutical dosage form of claim 15, wherein each azacitidine minitablet comprises about 20%-50% w/w of azacitidine, wherein the percentage by weight is relative to the weight of an uncoated azacitidine minitablet.

17. The pharmaceutical dosage form of claim 15, wherein each of the azacitidine minitablets comprises an intragranular layer and an extragranular layer.

18. The pharmaceutical dosage form of claim 17, wherein the intragranular layer comprises about 20%-50% w/w of azacitidine, about 10%-60% w/w of lactose monohydrate, about 2%-60% w/w of microcrystalline cellulose, about 1%-10% w/w of croscarmellose sodium, about 0.5%-10% w/w of hydroxypropyl methylcellulose (HPMC), about 0.1%-3% w/w/ of silicon dioxide, and about 0.1%-3% w/w of magnesium stearate, wherein the percentage by weight is relative to the total weight of the uncoated azacitidine minitablets.

19. The pharmaceutical dosage form of claim 17, wherein the extragranular layer comprises about 1%-60% w/w of microcrystalline cellulose, about 1%-10% w/w of croscarmellose sodium, about 0.1%-3% w/w silicon dioxide, and about 0.1%-3% w/w of magnesium stearate, wherein the percentage by weight is relative to the total weight of the uncoated azacitidine minitablets.

20. The pharmaceutical dosage form of claim 15, wherein the enteric coat comprises polymethacrylate or copolymers thereof.

21. The pharmaceutical dosage form of claim 15, wherein the enteric coat is sensitive to pH variations in intestine.

22. The pharmaceutical dosage form of claim 15, wherein each azacitidine minitablet further comprises a seal coat.

23. The pharmaceutical dosage form of claim 22, wherein the seal coat comprises hydroxypropylmethyl cellulose (HPMC).

24. The pharmaceutical dosage form of claim 15, comprising azacitidine and cedazuridine in the weight ratio of from about 1:1 to about 1:3.

25. The pharmaceutical dosage form of claim 15, comprising about 60 mg to 100 mg of azacitidine.

26. The pharmaceutical dosage form of claim 15, wherein the cedazuridine is in the form of uncoated minitablets, tablet, powder, blend, granules, or pellets.

27. The pharmaceutical dosage form of claim 26, wherein the cedazuridine minitablets, tablet, powder, blend, granules, or pellets comprises about 10%-90% w/w of cedazuridine, wherein the percentage by weight is relative to the total weight of the cedazuridine uncoated minitablets, tablet, powder, blend, granules, or pellets.

28. The pharmaceutical dosage form of claim 26, wherein the cedazuridine minitablets, tablet, powder, blend, granules, or pellets further comprising about one or more selected from the group consisting of: lactose monohydrate, hydroxypropyl methylcellulose (HPMC), croscarmellose sodium, silicon dioxide, and magnesium stearate.

29. The pharmaceutical dosage form of claim 15, comprising about 5 mg to 60 mg of cedazuridine.

30. A capsule comprising the pharmaceutical dosage form of claim 1.

31. A capsule comprising:
one or more azacitidine minitablets formulated for modified release, comprising azacitidine or a pharmaceutically acceptable salt thereof, pharmaceutically acceptable excipients, and an enteric coat; and
immediate release cedazuridine in a form of uncoated powder or blends, comprising cedazuridine or a pharmaceutically acceptable salt thereof and pharmaceutically acceptable excipients.

32. A method of treating cancer in a patient comprising administering the dosage form of claim 1, to the patient in need thereof.

33. The method of claim 32, wherein the cancer is leukemia.

34. The method of claim 32, wherein the cancer is selected from the group consisting of previously treated or untreated, de novo or secondary myelodysplastic syndromes (MDS), previously treated or untreated, de novo or secondary chronic myelomonocytic leukemia (CMML), acute myeloid leukemia (AML) and chronic myeloid leukemia (CML), malignant peripheral nerve sheath tumors (MPNST), neurological cancer, breast cancer, hormone receptor positive tumor, head and neck cancer, primary central chondrosarcoma, myeloproliferative neoplasm (MPN), recurrent B-cell non-Hodgkin lymphoma, recurrent diffuse large B-cell lymphoma, recurrent Hodgkin lymphoma, relapsed/refractory multiple myeloma (RRMM), metastatic colorectal cancer (mCRC), metastatic castration-resistant prostate cancer (mCRPC), and lung cancer.

35. The method of claim 32, wherein the cancer is selected from the group consisting of previously treated or untreated, de novo or secondary myelodysplastic syndromes (MDS), previously treated or untreated, de novo or secondary chronic myelomonocytic leukemia (CMML), acute myeloid leukemia (AML), and chronic myeloid leukemia (CML).

36. The method of claim 32, wherein the cancer is associated with refractory anemia, refractory anemia with ringed sideroblasts, or refractory anemia with excess blasts.

37. The method of claim 32, further comprising administering another therapeutic agent.

38. The method of claim 37, wherein the another therapeutic agent is one or more selected from the list consisting of: ADI-PEG 20, AMG-176, APG-115, APR-246, avelumab, bendamustine, bisantrene, brentuximab vedotin, capecitabine, CB-839, cisplatin, CS-01, cusatuzumab, cyclophosphamide, cytarabine, dasatinib, daunorubicin, DCLL9718S, decitabine, deferasirox, dexamethasone, durvalumab, eltrombopag, enasidenib, entinostat, entrectinib, enzalutamide, epacadostat, erythropoetin, etoposide, evorpacept, filgrastim, fludarabine phosphate, flumatinib, gemcitabine, gemtuzumab ozogamicin, gilteritinib, GM-CSF, GSK2879552, HMPL-523, homoharringtonine, IBI188, ibrutinib, idarubicin, itacitinib, ivosidenib, jaktinib, KPT-8602, LDE255, lenalidomide, lirilumab, LP-108, magrolimab, MAX-40279, mitoxantrone, mitoxantrone liposome, mocetinostat, moxifloxacin, nivolumab, olutasidenib, omacetaxine, oxaliplatin, paclitaxel, pembrolizumab, pevonedistat, pinometostat, pracinostat, quizartinib, revlimid, rigosertib, rituximab, romidepsin, RP7214, S64315, S65487, sabatolimab, seclidemstat, selumetinib, siremadlin, sirolimus, SL-401, SNDX-5613, sorafenib, talazoparib, tamibarotene, tolinapant, trastuzumab, tucidinostat, tyrosine kinase inhibitor, uproleselan, velcade, venetoclax, vincristine, visilizumab, vorinostat, and vosaroxin.

39-50. (canceled)
